(19) United States
(12) Patent Application Publication
(10) Pub. No.: US 2025/0109206 A1
(43) Pub. Date: Apr. 3, 2025

(54) ACTIVATABLE BISPECIFIC ANTI-CD3 AND ANTI-PD-L1 PROTEINS AND USES THEREOF

(71) Applicant: CENTESSA PHARMACEUTICALS (UK) LIMITED, Altrincham (GB)

(72) Inventors: William James Jonathan FINLAY, Glasgow (GB); Kristina GEGENBAUER, Lucan (IE)

(21) Appl. No.: 18/889,571

(22) Filed: Sep. 19, 2024

Related U.S. Application Data
(63) Continuation of application No. PCT/EP2023/058418, filed on Mar. 30, 2023.
(60) Provisional application No. 63/325,437, filed on Mar. 30, 2022.

(51) Int. Cl. C07K 16/28; A61K 39/00; A61P 35/00

(52) U.S. Cl. CPC C07K 16/2827; A61P 35/00; C07K 16/2809; A61K 2039/505; C07K 2317/31; C07K 2317/33; C07K 2317/522; C07K 2317/524; C07K 2317/526; C07K 2317/53; C07K 2317/565; C07K 2317/92

(57) ABSTRACT

Provided herein are protein molecules that specifically bind PD-L1 and also exhibit activatable specific CD3 binding in diseased tissues. Further provided herein are uses of such protein molecules to treat cancer.

### CROSS REFERENCE TO RELATED APPLICATIONS

This application is a continuation application of International Application No. PCT/EP2023/058418, filed on Mar. 30, 2023, which claims the benefit of priority to U.S. Provisional Application No. 63/325,437, filed on Mar. 30, 2022, the contents of which are hereby incorporated by reference in their entirety.

### REFERENCE TO AN ELECTRONIC SEQUENCE LISTING

The contents of the electronic sequence listing (ULSL_004_01WO_SeqList_ST26.xml; Size: 173,185 bytes; and Date of Creation: Mar. 29, 2023) are herein incorporated by reference in their entirety.

### TECHNICAL FIELD

The disclosure relates to activatable bispecific proteins and treatments for cancer.

### BACKGROUND

In immune oncology therapy, few of the key drug targets are exclusively expressed in diseased tissue, with the majority also being expressed in non-diseased tissue. In addition, many drugs employed in cancer treatment employ highly potent cell-killing mechanisms of action. As a result, engagement of the target by the drug in non-diseased tissue often causes unwanted side effects.

PD-L1 is a cell surface receptor that is a member of the immunoglobulin superfamily and is principally expressed on myeloid cells and regulatory T (Treg) cells in non-diseased tissues. However, PD-L1 has also been observed to be highly expressed on some cancer cells. PD-L1 binds to the membrane protein PD1. The interaction of PD-L1 with PD1 on T cells down-regulates T cell inflammatory activity, which promotes immune self-tolerance. PD-L1 is, therefore, described as an immune checkpoint. Hence, antagonistic anti-PD-L1 monoclonal antibodies that block interaction with PD1 have demonstrated the potential to act as well-tolerated immunotherapeutic agents in disease settings such as cancer, by “liberating” anti-cancer T cell responses from natural immune restriction. As such, PD-L1 is a drug target used to amplify the adaptive immune system's anti-cancer effects. In many cases, however, tumors may use multiple ‘escape mechanisms’ to negate the effects of PD-L1 agents, such as the downregulation of MHC class 1 and downregulation of cancer neoantigen expression.

CD3 (cluster of differentiation 3) is a T cell co-receptor that is involved in activating both CD8+ and CD4+ T cells as part of the T cell receptor complex. It is composed of four distinct chains, including two CD3ε chains. Historically, antibodies have been generated against the CD3ε chain which, upon binding, can induce a TCR activation signal in T lymphocytes which may lead to increased cytolytic activity against infected or cancerous cells. Using binding domains from these antibodies, CD3-ligating bispecific agents have also been generated that may direct T cell killing activity to target-specific cell classes without the need for TCR recognition of MHC-presented antigens on the target cells. Anti-PD-L1 antibodies could therefore be made more potent and broadly-acting therapeutic agents by gaining the ability to also bind CD3ε and thereby strongly engage T cell killing mechanisms in PD-L1 antibody-resistant disease settings. This would combine a key checkpoint inhibitor function with an inducible “synthetic immunity” that can synergistically stimulate the adaptive immune system. However, the ability to make this combination work in a single therapeutic agent structure (e.g., in standard bispecific antibody format with fully active PD-L1 and CD3 binding domains) is limited by the relatively broad expression profiles of both PD-L1 and CD3 on many cell types such as T cells and myeloid cells, as well as others. This broad expression profile may lead not only to dose-limiting toxicities for PD-L1/CD3 binding agents, but also to profound peripheral sink/biodistribution problems that limit the ability of such agents to achieve high enough exposure in diseased tissues to exploit the combined mechanisms. There is, therefore, a need for engineered forms of bispecific binding proteins with activity specifically targeted to the diseased tissue environment.

### SUMMARY

Provided herein is a protein comprising a first polypeptide chain comprising a heavy chain and a second polypeptide chain comprising a light chain, wherein the heavy chain comprises, in N-terminus to C-terminus order, an anti-PD-L1 heavy chain variable (VH) domain, a first CH1 domain, a first linker, an anti-CD3 VH domain, and a second CH1 domain; and wherein the light chain comprises, in N-terminus to C-terminus order, an anti-PD-L1 light chain variable (VL) domain, a first immunoglobulin light chain constant region, a second linker, an anti-CD3 VL domain, and a second immunoglobulin light chain constant region.

Provided herein is a protein comprising a first polypeptide chain comprising a heavy chain and a second polypeptide chain comprising a light chain, wherein the heavy chain comprises, in N-terminus to C-terminus order, an anti-PD-L1 heavy chain variable (VH) domain, a first CH1 domain, a first linker, an anti-CD3 light chain variable (VL) domain, and a first immunoglobulin light chain constant region, and wherein the light chain comprises, in N-terminus to C-terminus order, anti-PD-L1 VL domain, a second immunoglobulin light chain constant region, a second linker, an anti-CD3 VH domain, and a second CH1 domain.

In some embodiments, the heavy chain comprises in N-terminus to C-terminus order, the anti-PD-L1 VH domain, the first CH1 domain, the first linker, the anti-CD3 VH domain, the second CH1 domain, a hinge, a CH2 domain, and a CH3 domain.

In some embodiments, the protein further comprises a third polypeptide chain comprising a hinge, a CH2 domain, and a CH3 domain. In some embodiments, the third polypeptide chain comprises the amino acid sequence of SEQ ID NO: 124.

In some embodiments, a protein provided herein further comprises a moiety that provides half-life extension. In some embodiments, the moiety that provides half-life extension is polyethylene glycol (PEG) or an albumin binding domain.

Provided herein is a protein comprising a first polypeptide chain comprising a heavy chain and a second polypeptide chain comprising a light chain, wherein the heavy chain comprises, in N-terminus to C-terminus order, an anti-PD-L1 heavy chain variable (VH) domain, a first CH1 domain, a first linker, an anti-CD3 VH domain, a second CH1 domain, a first hinge, a first CH2 domain, and a first CH3 domain; and wherein the light chain comprises, in N-terminus to C-terminus order, an anti-PD-L1 light chain variable (VL) domain, a first immunoglobulin light chain constant region, a second linker, an anti-CD3 VL domain, a second immunoglobulin light chain constant region, a second hinge, a second CH2 domain, and a second CH3 domain.

In some embodiments, the first linker comprises the amino acid sequence of any one of SEQ ID NOs: 1-12. In some embodiments, the second linker comprises the amino acid sequence of any one of SEQ ID NOs: 1-12.

In some embodiments, the anti-PD-L1 VH domain comprises a HCDR1 comprising the amino acid sequence of SEQ ID NO: 20, a HCDR2 comprising the amino acid sequence of SEQ ID NO: 21, and a HCDR3 comprising the amino acid sequence of SEQ ID NO: 22; and the anti-PD-L1 VL domain comprises a LCDR1 comprising the amino acid sequence of SEQ ID NO: 23, a LCDR2 comprising the amino acid sequence of SEQ ID NO: 24, and a LCDR3 comprising the amino acid sequence of SEQ ID NO: 25.

In some embodiments, the anti-CD3 VH domain comprises a HCDR1 comprising the amino acid sequence of SEQ ID NO: 26, a HCDR2 comprising the amino acid sequence of SEQ ID NO: 27, and a HCDR3 comprising the amino acid sequence of SEQ ID NO: 29; and the anti-CD3 VL domain comprises a LCDR1 comprising the amino acid sequence of SEQ ID NO: 30, a LCDR2 comprising the amino acid sequence of SEQ ID NO: 31, and a LCDR3 comprising the amino acid sequence of SEQ ID NO: 32.

In some embodiments, the anti-CD3 VH domain comprises a HCDR1 comprising the amino acid sequence of SEQ ID NO: 26, a HCDR2 comprising the amino acid sequence of SEQ ID NO: 28, and a HCDR3 comprising the amino acid sequence of SEQ ID NO: 29; and the anti-CD3 VL domain comprises a LCDR1 comprising the amino acid sequence of SEQ ID NO: 30, a LCDR2 comprising the amino acid sequence of SEQ ID NO: 31, and a LCDR3 comprising the amino acid sequence of SEQ ID NO: 32.

In some embodiments, the anti-PD-L1 VH domain comprises a HCDR1 comprising the amino acid sequence of SEQ ID NO: 20, a HCDR2 comprising the amino acid sequence of SEQ ID NO: 21, and a HCDR3 comprising the amino acid sequence of SEQ ID NO: 22; the anti-PD-L1 VL domain comprises a LCDR1 comprising the amino acid sequence of SEQ ID NO: 23, a LCDR2 comprising the amino acid sequence of SEQ ID NO: 24, and a LCDR3 comprising the amino acid sequence of SEQ ID NO: 25; the anti-CD3 VH domain comprises a HCDR1 comprising the amino acid sequence of SEQ ID NO: 26, a HCDR2 comprising the amino acid sequence of SEQ ID NO: 27, and a HCDR3 comprising the amino acid sequence of SEQ ID NO: 29; and the anti-CD3 VL domain comprises a LCDR1 comprising the amino acid sequence of SEQ ID NO: 30, a LCDR2 comprising the amino acid sequence of SEQ ID NO: 31, and a LCDR3 comprising the amino acid sequence of SEQ ID NO: 32.

In some embodiments, the anti-PD-L1 VH domain comprises a HCDR1 comprising the amino acid sequence of SEQ ID NO: 20, a HCDR2 comprising the amino acid sequence of SEQ ID NO: 21, and a HCDR3 comprising the amino acid sequence of SEQ ID NO: 22; the anti-PD-L1 VL domain comprises a LCDR1 comprising the amino acid sequence of SEQ ID NO: 23, a LCDR2 comprising the amino acid sequence of SEQ ID NO: 24, and a LCDR3 comprising the amino acid sequence of SEQ ID NO: 25; the anti-CD3 VH domain comprises a HCDR1 comprising the amino acid sequence of SEQ ID NO: 26, a HCDR2 comprising the amino acid sequence of SEQ ID NO: 28, and a HCDR3 comprising the amino acid sequence of SEQ ID NO: 29; and the anti-CD3 VL domain comprises a LCDR1 comprising the amino acid sequence of SEQ ID NO: 30, a LCDR2 comprising the amino acid sequence of SEQ ID NO: 31, and a LCDR3 comprising the amino acid sequence of SEQ ID NO: 32.

In some embodiments, the anti-PD-L1 VH domain comprises the amino acid sequence of any one of SEQ ID NOs: 33 and 54-107. In some embodiments, the anti-PD-L1 VL domain comprises the amino acid sequence of SEQ ID NO: 34.

In some embodiments, the anti-PD-L1 VH domain comprises the amino acid sequence of any one of SEQ ID NOs: 33 and 54-107, and the anti-PD-L1 VL domain comprises the amino acid sequence of SEQ ID NO: 34.

In some embodiments, the anti-CD3 VH domain comprises the amino acid sequence of any one of SEQ ID NOs: 44-48. In some embodiments, the anti-CD3 VL domain comprises the amino acid sequence of any one of SEQ ID NOs: 37-42.

In some embodiments, the heavy chain comprises the amino acid sequence of any one of SEQ ID NOs: 116-123, 129-132, 137-141, 142, 144, 146, 147, 148, 150, and 152. In some embodiments, the light chain comprises the amino acid sequence of any one of SEQ ID NOs: 53, 108-115, 125-128, and 133-136, 143, 145, 151, and 153.

Provided herein is a protein wherein:
(a) the heavy chain comprises the amino acid sequence of SEQ ID NO: 122, the light chain comprises the amino acid sequence of SEQ ID NO: 114, and the third polypeptide chain comprises the amino acid sequence of SEQ ID NO: 124;
(b) the heavy chain comprises the amino acid sequence of SEQ ID NO: 123, the light chain comprises the amino acid sequence of SEQ ID NO: 115, and the third polypeptide chain comprises the amino acid sequence of SEQ ID NO: 124;
(c) the heavy chain comprises the amino acid sequence of SEQ ID NO: 116, the light chain comprises the amino acid sequence of SEQ ID NO: 108, and the third polypeptide chain comprises the amino acid sequence of SEQ ID NO: 124;
(d) the heavy chain comprises the amino acid sequence of SEQ ID NO: 117, the light chain comprises the amino acid sequence of SEQ ID NO: 109, and the third polypeptide chain comprises the amino acid sequence of SEQ ID NO: 124;
(e) the heavy chain comprises the amino acid sequence of SEQ ID NO: 118, the light chain comprises the amino acid sequence of SEQ ID NO: 108, and the third polypeptide chain comprises the amino acid sequence of SEQ ID NO: 124;
(f) the heavy chain comprises the amino acid sequence of SEQ ID NO: 119, the light chain comprises the amino acid sequence of SEQ ID NO: 109, and the third polypeptide chain comprises the amino acid sequence of SEQ ID NO: 124;
(g) the heavy chain comprises the amino acid sequence of SEQ ID NO: 120, the light chain comprises the amino acid sequence of SEQ ID NO: 108, and the third polypeptide chain comprises the amino acid sequence of SEQ ID NO: 124;
(h) the heavy chain comprises the amino acid sequence of SEQ ID NO: 116, the light chain comprises the amino acid sequence of SEQ ID NO: 110, and the third polypeptide chain comprises the amino acid sequence of SEQ ID NO: 124;
(i) the heavy chain comprises the amino acid sequence of SEQ ID NO: 118, the light chain comprises the amino acid sequence of SEQ ID NO: 110, and the third polypeptide chain comprises the amino acid sequence of SEQ ID NO: 124;
(j) the heavy chain comprises the amino acid sequence of SEQ ID NO: 120, the light chain comprises the amino acid sequence of SEQ ID NO: 110, and the third polypeptide chain comprises the amino acid sequence of SEQ ID NO: 124;
(k) the heavy chain comprises the amino acid sequence of SEQ ID NO: 116, the light chain comprises the amino acid sequence of SEQ ID NO: 111, and the third polypeptide chain comprises the amino acid sequence of SEQ ID NO: 124;
(l) the heavy chain comprises the amino acid sequence of SEQ ID NO: 118, the light chain comprises the amino acid sequence of SEQ ID NO: 111, and the third polypeptide chain comprises the amino acid sequence of SEQ ID NO: 124;
(m) the heavy chain comprises the amino acid sequence of SEQ ID NO: 120, the light chain comprises the amino acid sequence of SEQ ID NO: 111, and the third polypeptide chain comprises the amino acid sequence of SEQ ID NO: 124;
(n) the heavy chain comprises the amino acid sequence of SEQ ID NO: 116, the light chain comprises the amino acid sequence of SEQ ID NO: 112, and the third polypeptide chain comprises the amino acid sequence of SEQ ID NO: 124;
(o) the heavy chain comprises the amino acid sequence of SEQ ID NO: 116, the light chain comprises the amino acid sequence of SEQ ID NO: 113, and the third polypeptide chain comprises the amino acid sequence of SEQ ID NO: 124;
(p) the heavy chain comprises the amino acid sequence of SEQ ID NO: 121, the light chain comprises the amino acid sequence of SEQ ID NO: 108, and the third polypeptide chain comprises the amino acid sequence of SEQ ID NO: 124;
(q) the heavy chain comprises the amino acid sequence of SEQ ID NO: 131, the light chain comprises the amino acid sequence of SEQ ID NO: 127, and the third polypeptide chain comprises the amino acid sequence of SEQ ID NO: 124; or
(r) the heavy chain comprises the amino acid sequence of SEQ ID NO: 120, the light chain comprises the amino acid sequence of SEQ ID NO: 127, and the third polypeptide chain comprises the amino acid sequence of SEQ ID NO: 124.

In some embodiments, the heavy chain comprises an IgG, IgE, IgM, IgD, IgA, or IgY constant region. In some embodiments, the heavy chain comprises an IgG1, IgG2, IgG3, IgG4, IgA1 or IgA2 constant region. In some embodiments, the heavy chain comprises an immunologically inert constant region. In some embodiments, the heavy chain comprises a wild-type human IgG1 constant region, a human IgG1 constant region comprising the amino acid substitutions L234A, L235A and G237A, a wild-type human IgG2 constant region, a wild-type human IgG4 constant region, or a human IgG4 constant region comprising the amino acid substitution S228P, wherein numbering is according to the EU index as in Kdbat.

Provided herein is an immunoconjugate comprising a protein disclosed herein, linked to a therapeutic agent. In some embodiments, the therapeutic agent is a cytotoxin, a radioisotope, a chemotherapeutic agent, an immunomodulatory agent, a cytostatic enzyme, a cytolytic enzyme, a therapeutic nucleic acid, an anti-angiogenic agent, an anti-proliferative agent, or a pro-apoptotic agent.

Provided herein is a pharmaceutical composition comprising a protein or an immunoconjugate disclosed herein, and a pharmaceutically acceptable carrier.

Provided herein is a nucleic acid molecule encoding (a) the first polypeptide chain amino acid sequence; (b) the second polypeptide chain amino acid sequence; or (c) both the first polypeptide chain and the second polypeptide chain amino acid sequences of a protein disclosed herein.

Provided herein is a nucleic acid molecule encoding (a) the heavy chain amino acid sequence; (b) the light chain amino acid sequence; or (c) both the heavy chain and the light chain amino acid sequences of a protein disclosed herein.

Provided herein is an expression vector comprising a nucleic acid molecule disclosed herein.

Provided herein is a recombinant host cell comprising a nucleic acid molecule or an expression vector disclosed herein.

Provided herein is a method of producing a protein, the method comprising: culturing a recombinant host cell disclosed herein under conditions whereby the nucleic acid molecule is expressed, thereby producing the protein; and isolating the protein from the host cell or culture.

Provided herein is a method for enhancing an anti-cancer immune response in a subject, the method comprising administering to the subject a therapeutically effective amount of a protein, an immunoconjugate, or a pharmaceutical composition disclosed herein.

Provided herein is a method for treating cancer in a subject, the method comprising administering to the subject a therapeutically effective amount of a protein, an immunoconjugate, or a pharmaceutical composition disclosed herein.

Provided herein is a method for ameliorating a symptom of cancer in a subject, the method comprising administering to the subject a therapeutically effective amount of a protein, an immunoconjugate, or a pharmaceutical composition disclosed herein.

Provided herein is a protein, an immunoconjugate, or a pharmaceutical composition disclosed herein, for use in enhancing an anti-cancer immune response in a subject.

Provided herein is a protein, an immunoconjugate, or a pharmaceutical composition disclosed herein, for use in treating cancer in a subject.

Provided herein is a protein, an immunoconjugate, or a pharmaceutical composition disclosed herein, for use in ameliorating a symptom of cancer in a subject.

In some embodiments of the methods and uses provided herein, the cancer is gastrointestinal stromal cancer (GIST), pancreatic cancer, skin cancer, melanoma, breast cancer, lung cancer, bronchial cancer, colorectal cancer, prostate cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of the oral cavity or pharynx, liver cancer, kidney cancer, renal cell carcinoma, testicular cancer, biliary tract cancer, small bowel cancer, appendix cancer, salivary gland cancer, thyroid cancer, adrenal gland cancer, osteosarcoma, chondrosarcoma, or cancer of hematological tissues.

### BRIEF DESCRIPTION OF THE DRAWINGS

FIG. 1A depicts a diagram of a protein molecule disclosed herein in intact (left), activated protease-cleaved (middle) and deactivated protease-cleaved (right) conformations. In an intact conformation, the PD-L1 Fab binding domain is exposed and able to bind their cognate target. The CD3 Fab domain is inhibited from binding by linkers in both the heavy and light chains that are both proteolytically cleavable and may be sequentially cleaved by matrix metalloproteases (MMPs) and/or cathepsins. A first cleavage event creates an intermediate active state which allows both PD-L1 and CD3 Fabs from a single protein construct to bind their cognate targets, thereby potentially directing killing of PD-L1+ cells by directed activation of CD3+ T cells. A second cleavage dissociates the PD-L1 and CD3 Fabs, removing the ability of a single molecule to bind both PD-L1 and CD3 simultaneously.

FIG. 1B depicts a detailed diagram of an intact asymmetric LB (LockBody) protein consisting of heavy chain, light chain and Fc stump. Each light chain is comprised of two Fabs linked by a lower hinge linker (LHL). Each heavy chain is comprised of two Fabs linked by a lower hinge linker (LHL) an Fc hinge and Fc fragment containing both CH2 and CH3 domains with either N-linked glycosylation sites (CH2) or Knobs mutations (CH3). Finally, the asymmetric LB protein contains a third polypeptide which contains both CH2 and CH3 domains with either N-linked glycosylation sites (CH2) or Holes mutations (CH3).

FIG. 1C depicts diagrams of alternative formats for the protein molecules disclosed herein, where the molecule may; lack an Fc fragment (left); may lack an Fc fragment but achieve half-life extension (HLE) through an alternative mechanism such as PEGylation, addition of an albumin binding domain, etc. (center); or may contain an Fc fragment but be constructed from only two polypeptides (right) as symmetric one-arm constructs. Each polypeptide chain contains either two light chain Fabs linked by lower hinge linkers, an Fc hinge without disulfide bonds and Fc fragment (CH2 and CH3 domains) or two heavy chain Fabs linked by lower hinge linkers, an Fc hinge without disulfide bonds and Fc fragment (CH2 and CH3 domains).

FIG. 1D depicts detailed diagrams of asymmetric one-arm constructs in which the two polypeptide chains contain one light chain and one heavy chain Fab linked by LHL. As above, each polypeptide chain further contains an Fc hinge without disulfide bonds and Fc fragment (CH2 and CH3 domains).

FIG. 2 depicts a diagram of one proposed mechanism of activity of activatable bispecific protein molecules provided herein.

FIG. 3A-FIG. 3G depict activity of (1) IgG1 isotype, (2) a commercial CD3×Her2 Bispecific T cell Engager (BiTE) used as a positive control anti-Her2/anti-CD3 bispecific antibody as indicated or (3) test articles (before or after treatment with MMP12 for 0, 0.5 hour, 1 hour or 4 hours) in a cell-based assay that measures the ability of agents to bind hPD-L1 on MDA-MB231 cancer cells and induce CD3 signaling on co-cultured Jurkat cells (measured in fold activation). FIG. 3A depicts activity of IgG1 isotype, CD3×Her2 BiTE used as a positive control and test article LB204 (before or after treatment with MMP12 for 0 or 4 hours). LB204 induces low CD3 signal before MMP12 treatment and moderate CD3 fold activation after MMP12 treatment that stays below levels induced by the commercial CD3×Her2 BiTE control. FIG. 3B depicts activity of IgG1 isotype, CD3×Her2 BiTE used as a positive control and test article LB206 (before or after treatment with MMP12 for 0 or 4 hours). LB206 induces low CD3 signal before MMP12 treatment and high CD3 signal after treatment with MMP12 with higher levels compared to commercial CD3×Her2 BiTE control. FIG. 3C depicts activity of IgG1 isotype, CD3×Her2 BiTE used as a positive control and test article LB208 (before or after treatment with MMP12 for 0 or 4 hours). LB208 induces no CD3 signal before MMP12 treatment and moderate CD3 fold activation after MMP12 treatment that stays below levels induced by the commercial CD3×Her2 BiTE control. FIG. 3D depicts activity of IgG1 isotype, CD3×Her2 BiTE used as a positive control and test article LB209 (before or after treatment with MMP12 for 0 or 4 hours). LB209 induces no CD3 signal before MMP12 treatment and moderate CD3 fold activation after MMP12 treatment that stays below levels induced by the commercial CD3×Her2 BiTE control. FIG. 3E depicts activity of IgG1 isotype, CD3×Her2 BiTE used as a positive control and test article LB210 (before or after treatment with MMP12 for 0 or 4 hours). LB210 induces no CD3 signal before MMP12 treatment or after. FIG. 3F depicts activity of IgG1 isotype, CD3×Her2 BiTE used as a positive control and test article LB213 (before or after treatment with MMP12 for 0 or 4 hours). LB213 induces no CD3 signal before MMP12 treatment and relatively high CD3 signal after treatment with MMP12 but staying below levels induced by the commercial CD3×Her2 BiTE control. FIG. 3G depicts activity of IgG1 isotype, test article LB205 (before or after treatment with MMP12 for 0, 0.5 or 1 hour). LB205 induces no CD3 signal before MMP12 treatment and high CD3 signal after treatment with MMP12 for either 0.5 hour or 1 hour.

FIG. 4A-FIG. 4D depict the characterization (SPR, ELISA, and Jurkat activity) of Fab and IgG formats of two CD3 humanization variants. FIG. 4A depicts surface plasmon resonance (SPR) binding of each V domain humanization variant (in monovalent Fab format) to human recombinant CD3 δε heterodimer. FIG. 4B depicts surface plasmon resonance (SPR) binding of each V domain humanization variant (in monovalent Fab format) to cynomolgus (cyno) monkey recombinant CD3 Sc heterodimer. FIG. 4C shows IgG binding of IgG001 and IgG002 to human and cyno recombinant CD3 δε heterodimer in ELISA. FIG. 4D depicts CD3 dependent activation assay comparing emitted luciferase signals from Jurkat reporter cells stimulated with SP34 IgG or its respective humanization variants in IgG format.

FIG. 5A depicts MMP12 digested LB proteins run on SDS PAGE under reducing or non-reducing conditions. 2 μg/lane of exemplary LB proteins 204, 206, 208, 209, 210 and 213 previously incubated with MMP12 for 0 h and 4 h show differential digestion profiles. Notably, a prominent 25 kDa/50 kDa band in reducing/non-reducing SDS-PAGE respectively, resulting from light chain cleavage and disappearing intact heavy chains at 75 kDa in reducing conditions (and 150 kDa in non-reducing conditions) can be observed in concert, demonstrating that linkers in both the heavy and light chains become cleaved.

FIG. 5B depicts MMP12 digested LB proteins run on SDS PAGE under reducing or non-reducing conditions. 2 μg/lane of LB proteins LB206 and LB220 previously incubated with MMP12 for 0 h, 5 min, 15 min, 30 min, 60 min or 120 min show differential digestion profiles. Notably, a prominent 25 kDa/50 kDa band in reducing/non-reducing SDS-PAGE respectively, resulting from light chain cleavage and disappearing intact heavy chains at 75 kDa in reducing conditions (and 150 kDa in non-reducing conditions) can be observed in concert, demonstrating that linkers in both the heavy and light chains become cleaved. In direct comparison, LB protein LB220 appears to be cleaved at a faster rate by MMP12 than LB206.

FIG. 6A-FIG. 6C depict activity of IgG1 isotype or test articles (before or after treatment with MMP12 for 0, 0.5 or 1 hours) in a cell-based assay which measures the ability of test agents to bind hPD-L1 on MDA-MB231 cancer cells and induce CD3 signaling on co-cultured Jurkat cells (measured in fold activation). FIG. 6A depicts activity of IgG1 isotype, test article LB217 (before or after treatment with MMP12 for 0, 0.5 or 1 hour). LB217 induces low CD3 signal before MMP12 treatment and high CD3 signal after treatment with MMP12 for either 0.5 hour or 1 h hour. FIG. 6B depicts activity of IgG1 isotype, test article LB218 (before or after treatment with MMP12 for 0, 0.5 or 1 hour). LB217 induces no CD3 signal before MMP12 treatment and high CD3 signal after treatment with MMP12 for 0.5 hour. In contrast CD3 signal after treatment with MMP12 for 1 h hour is lower. FIG. 6C depicts activity of IgG1 isotype, test article LB220 (before or after treatment with MMP12 for 0, 0.5 or 1 hour). LB220 induces low CD3 signal before MMP12 treatment and high CD3 signal after treatment with MMP12 for 0.5 hour. In contrast, CD3 signal after treatment with MMP12 for 1 h hour is much lower, similar to no MMP12 treatment.

FIG. 7A-FIG. 7H depict ELISA binding of intact or up to 1 h MMP12 incubated LB proteins to human PD-L1 with atezolizumab as positive control or human and cyno CD3 δε heterodimer. The negative control protein used is human IgG1 isotype. FIG. 7A depicts ELISA binding of intact or 5 min MMP12 incubated LB206 protein to human PD-L1 with atezolizumab as positive control or IgG1 isotype as negative control (no signal). Intact and MMP12 treated LB206 bind to PD-L1 to a similar level. FIG. 7B depicts ELISA binding of intact or 5 min MMP12 incubated LB206 protein to human CD3 δε heterodimer or IgG1 isotype as negative control (no signal). Intact LB206 binds to human CD3 δε heterodimer to very low level. In contrast, MMP12 treated LB206 binds strongly to human CD3 δε heterodimer. FIG. 7C depicts ELISA binding of intact or 5 min, 15 min, 30 min or 60 min MMP12 incubated LB218 protein to human PD-L1 with IgG1 isotype as negative control (no signal). Intact and 5 or 15 min MMP12 treated LB218 bind to PD-L1 to a similar level. PD-L1 appears lower for 30 min and 60 min. FIG. 7D depicts ELISA binding of intact or 5 min, 15 min, 30 min or 60 min MMP12 incubated LB218 protein to human CD3 δε heterodimer with IgG1 isotype as negative control (no signal). Intact LB218 shows binding to human CD3 SC heterodimer to very low level. With increasing MMP12 treatment times, higher binding to human CD3 δε heterodimer can be observed. 60 min MMP12 treated LB218 shows highest binding to human CD3 δε heterodimer. FIG. 7E depicts ELISA binding of intact or 5 min, 15 min, 30 min or 60 min MMP12 incubated LB218 protein to cyno CD3 Sc heterodimer with IgG1 isotype as negative control (no signal). Intact LB218 shows binding to cyno CD3 δε heterodimer to very low level. With increasing MMP12 treatment times, higher binding to cyno CD3 δε heterodimer can be observed. 60 min MMP12 treated LB218 shows highest binding to cyno CD3 δε heterodimer. FIG. 7F depicts ELISA binding of intact or 5 min, 15 min, 30 min or 60 min MMP12 incubated LB213 protein to human PD-L1 with IgG1 isotype as negative control (no signal). Intact and 5 or 15 min MMP12 treated LB213 bind to PD-L1 to a similar level. While binding to PD-L1 appears lower for 30 min and 60 min, suggesting occurrence of protein cleavage 2 (FIG. 1A). FIG. 7G depicts ELISA binding of intact or 5 min, 15 min, 30 min or 60 min MMP12 incubated LB213 protein to human CD3 δε heterodimer with IgG1 isotype as negative control (no signal). Intact LB213 shows binding to human CD3 SC heterodimer to very low level. With increasing MMP12 treatment times, higher binding to human CD3 δε heterodimer can be observed. 60 min MMP12 treated LB213 shows highest binding to human CD3 δε heterodimer. FIG. 7H depicts ELISA binding of intact or 5 min, 15 min, 30 min or 60 min MMP12 incubated LB213 protein to cyno CD3 Sc heterodimer with IgG1 isotype as negative control (no signal). Intact LB213 shows binding to cyno CD3 δε heterodimer to very low level. With increasing MMP12 treatment times, higher binding to cyno CD3 δε heterodimer can be observed. 60 min MMP12 treated LB213 shows highest binding to cyno CD3 δε heterodimer.

FIG. 8A depicts Jurkat cell binding of intact or 0 to 2 h MMP12 incubated LB protein LB206. No binding to Jurkat cells can be observed for intact LB206 (0 min) or isotype control. In contrast, all MMP12 treated samples show similar binding levels to Jurkat cells. FIG. 8B depicts PDL-1 expression on three different cell lines after IFNγ stimulation-A549, MDA-MB-231 and RKO cells.

FIG. 9A-FIG. 9F depict primary T cell killing of cancer cell lines (A549, RKO, MDA-MB-231) mediated by intact or MMP12 incubated LB proteins with indicated concentrations as measured by the Incucyte® live cell analysis platform. FIG. 9A depicts primary T cell killing of cancer cell line A549 mediated by intact or 30 min MMP12 incubated LB 206 as measured by the Incucyte® live cell analysis platform.

FIG. 9B depicts primary T cell killing of cancer cell line A549 mediated by intact or 30 min MMP12 incubated LB 218 as measured by the Incucyte® live cell analysis platform. FIG. 9C depicts primary T cell killing of cancer cell line RKO mediated by intact or 30 min MMP12 incubated LB 206 as measured by the Incucyte® live cell analysis platform. FIG. 9D depicts primary T cell killing of cancer cell line RKO mediated by intact or 30 min MMP12 incubated LB 218 as measured by the Incucyte® live cell analysis platform. FIG. 9E depicts primary T cell killing of cancer cell line MDA-MB-231 mediated by intact or 30 min MMP12 incubated LB 206 as measured by the Incucyte® live cell analysis platform. FIG. 9F depicts primary T cell killing of cancer cell line MDA-MB-231 mediated by intact or 30 min MMP12 incubated LB 213 as measured by the Incucyte® live cell analysis platform.

FIG. 10A-FIG. 10E depict changes in tumor volume by caliper measurements (baseline corrected) or body weight changes in CD34+ myeloid boosted NCG mice with established MDA-MB-231 tumors over time while being treated with LB206, LB213, LB220, Atezolizumab or IgG isotype control. FIG. 10A shows tumor volume by caliper measurements over time of LB206, IgG isotype control or Atezolizumab treated CD34+ myeloid boosted NCG mice with established MDA-MB-231 tumors. All LB206 treated groups (4.5 mg/kg, 8.5 mg/kg, 12 mg/kg) show tumor regressions to a similar extent. FIG. 10B shows tumor volume by caliper measurements over time of LB220, IgG isotype control or Atezolizumab treated CD34+ myeloid boosted NCG mice with established MDA-MB-231 tumors. All LB220 treated groups (8.5 mg/kg, 12 mg/kg) show tumor regressions initially followed by tumor growth inhibition. FIG. 10C shows tumor volume by caliper measurements over time of LB213, IgG isotype control or Atezolizumab treated CD34+ myeloid boosted NCG mice with established MDA-MB-231 tumors. All LB213 treated groups show dose dependent tumor growth inhibition (4.5, 8.5 mg/kg, 12 mg/kg). FIG. 10D shows tumor volumes of individual tumors of either IgG isotype or LB206 treated CD34+ myeloid boosted NCG mice until Day 51. Dosing regimen is indicated by triangles. 5 out of 8 LB206 treated tumors remain regressed 33 days after treatment is stopped. FIG. 10E shows mean body weight changes of all treatment groups over time. No group showed body weight loss beyond 10% at any time.

### DETAILED DESCRIPTION

There are two key issues that restrict the efficacy in CD3-activating, tumor-targeting bispecific antibody drugs for oncology:

1) That the antibody target protein found on cancer cells (e.g., PD-L1) is potentially expressed on many different cell classes in the body, not just on the tumor cell. This off-tumor target expression often leads to dose-limiting side effect risks as the CD3 binding domain in a standard bispecific molecule is constitutively active and may therefore direct T cells to kill any target-positive cell, whether it is in diseased tissue or not. Off-tumor target expression may also lead to antigen “sink” effects, which reduces the amount of drug penetrating the tumor.

2) CD3-positive cells are found in the bloodstream and at high concentration in secondary lymphoid tissues, creating a large sink effect for this arm, affecting biodistribution and free drug availability for tumor penetration.

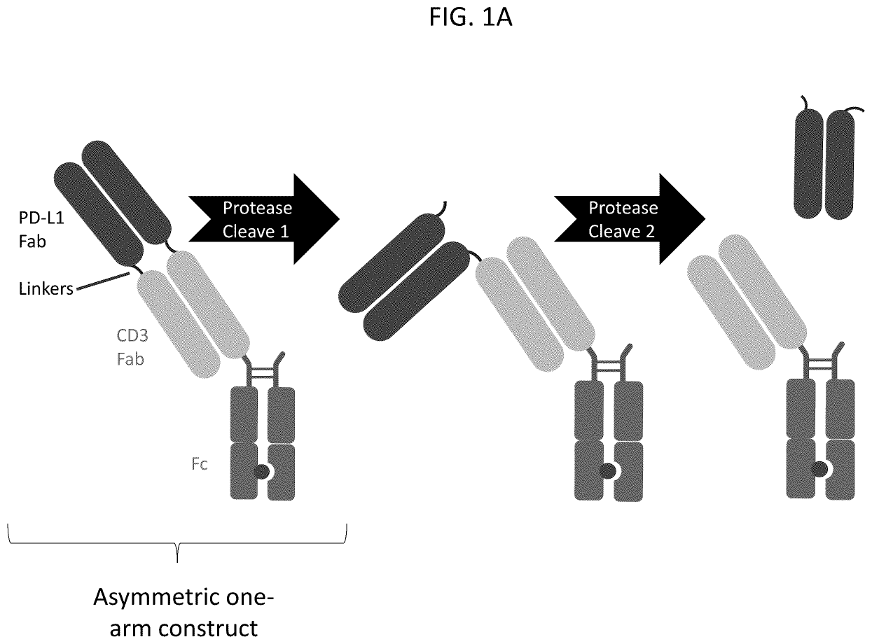

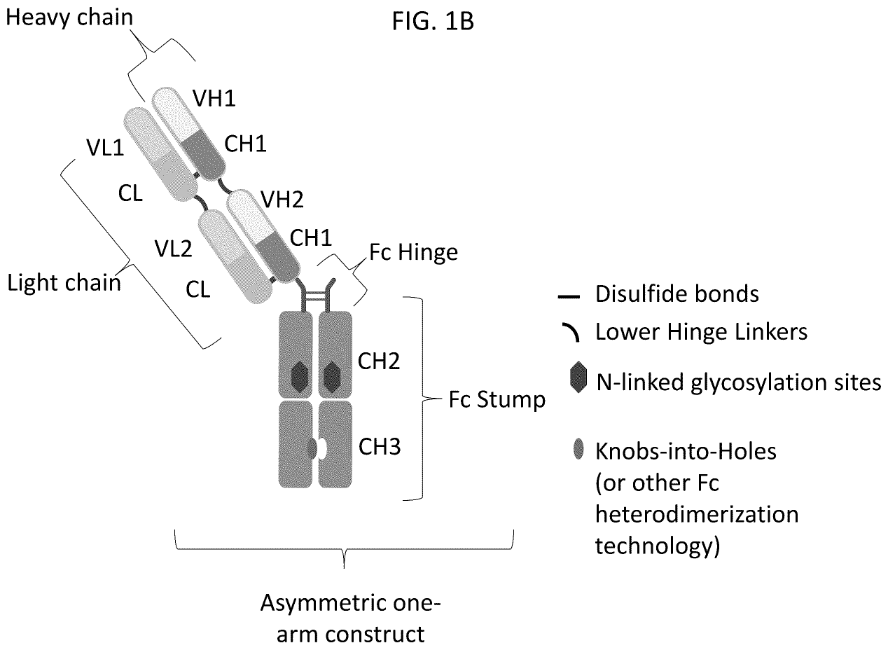

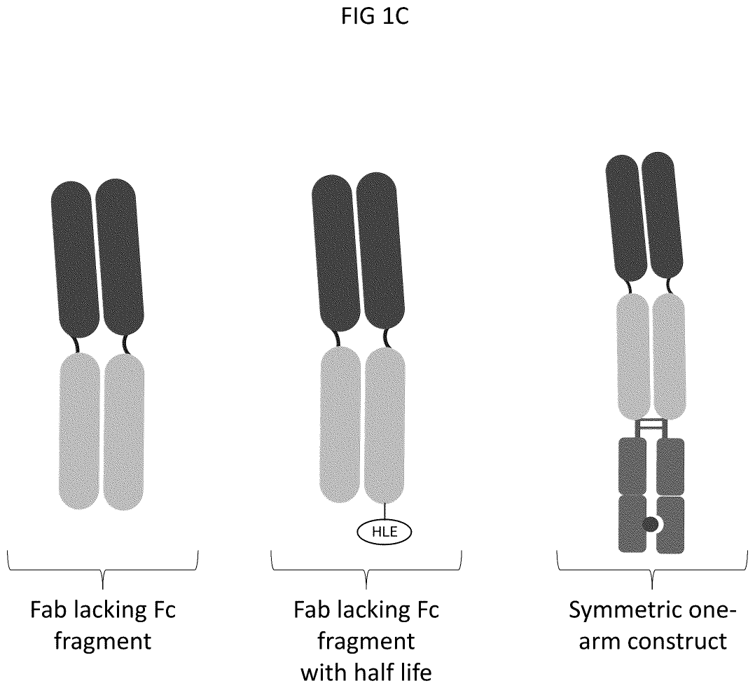

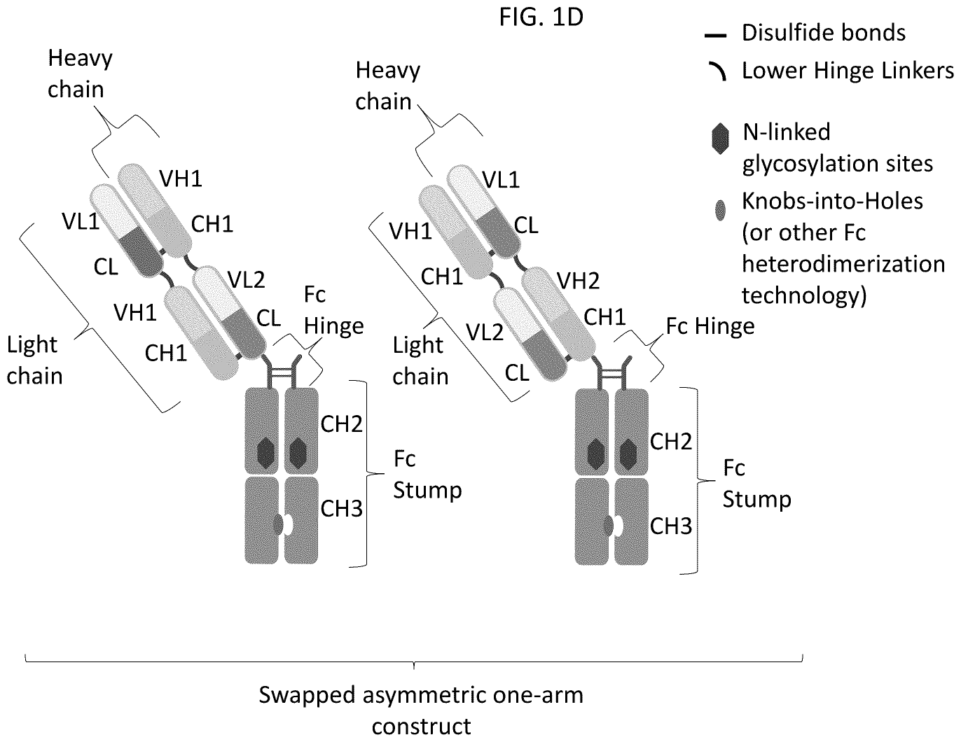

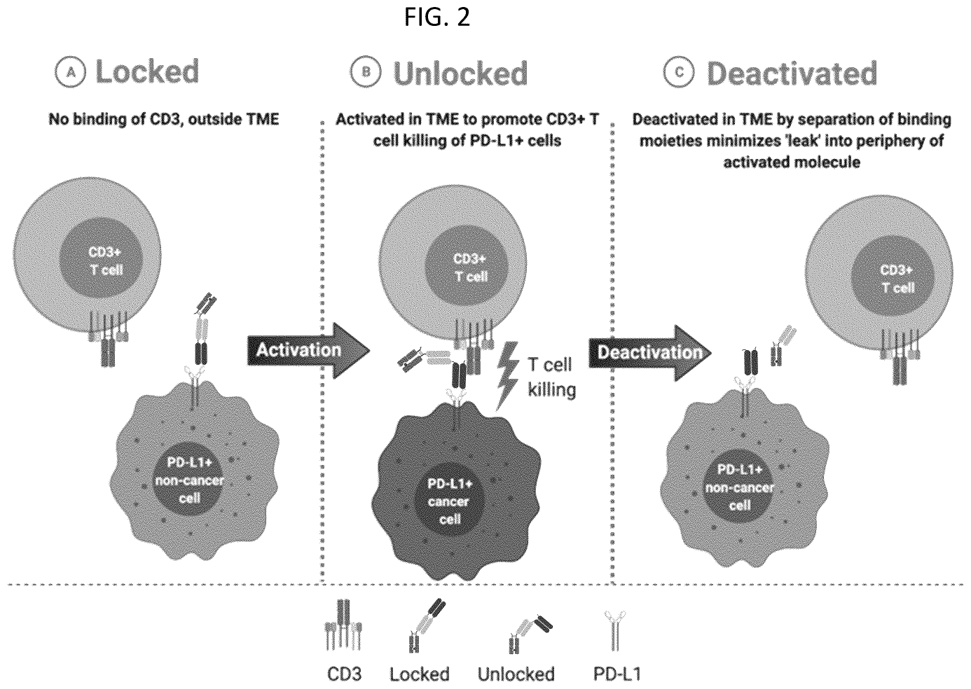

Both factors described above minimize the potential safety and efficacy of bispecific antibodies that drive CD3 engagement and activation. The anti-PD-L1 and anti-CD3 proteins (see, e.g., FIG. 1A-FIG. 1D) provided herein overcome the peripheral sink and toxicity issues by minimizing binding of CD3 outside of diseased tissue. This effect is achieved by adding PD-L1 binding domains and linkers above (i.e., amino-terminal to) the CD3 binding domains. The use of appropriate upper domain and linker combinations results in a configuration that minimizes binding activity in the lower (i.e., carboxy-terminal) CD3 domain. The PD-L1 domain then drives high concentration in PD-L1 enriched tumor microenvironments. The protein construct linker system exploits the elevated MMP and cathepsin activity that is common in solid tumors to cleave the linker peptides, exposing the CD3 binding domains and thereby conditionally activating the CD3-activating activity in the tumor, rather than the periphery. These combined biological functions thereby afford the molecule the potential to avoid the peripheral CD3 sink and maximize T cell immune responses to cancer cells, as outlined in FIG. 2.

Provided herein are proteins that are conditionally active in diseased human tissues. The proteins of the disclosure are fully active in specifically binding and blocking PD-L1 throughout the body; exhibit minimized binding of CD3 in healthy tissue; and become highly activated in CD3 binding and activation once in the PD-L1-positive diseased tissue environment. A protein of the disclosure comprises a CD3 binding domain that is masked by a PD-L1 binding domain in non-diseased tissues. The protein also comprises two peptide linkers that are cleaved by one or more proteases expressed in a diseased tissue (e.g., a tumor). The linker cleavage unmasks the CD3 binding domain in the diseased tissue, thus allowing binding and/or function of the protein selectively in the diseased tissue.

### PROTEIN MOLECULES

Provided herein are proteins comprising two Fab fragments (an anti-PD-L1 Fab and an anti-CD3 Fab). The protein is monovalent when in the intact structure and can only have a maximum of monovalent CD3 binding when activated, to minimize peripheral toxicity risk associated with bivalent, activating anti-CD3 antibodies.

In some embodiments, a protein comprises a first polypeptide chain comprising a heavy chain and a second polypeptide chain comprising a light chain, wherein the heavy chain comprises, in N-terminus to C-terminus order, an anti-PD-L1 heavy chain variable (VH) domain, a first CH1 domain, a first linker, an anti-CD3 VH domain, and a second CH1 domain; wherein the light chain comprises, in N-terminus to C-terminus order, an anti-PD-L1 light chain variable (VL) domain, a first immunoglobulin light chain constant region, a second linker, an anti-CD3 VL domain, and a second immunoglobulin light chain constant region. In some embodiments, the heavy chain further comprises an immunoglobulin hinge region and an Fc domain at its C-terminus. In some embodiments, the heavy chain comprises in N-terminus to C-terminus order, the anti-PD-L1 VH domain, the first CH1 domain, the first linker, the anti-CD3 VH domain, the second CH1 domain, a hinge, a CH2 domain, and a CH3 domain.

In some embodiments, the protein further comprises a third polypeptide chain comprising a hinge, a CH2 domain, and a CH3 domain. The third polypeptide chain may be referred to as a “Fc-stump”.

A diagram of an illustrative protein of the disclosure, with labeled domains, is shown in FIG. 1. FIG. 1A depicts a diagram of a protein molecule disclosed herein in intact (left), activated protease-cleaved (middle) and deactivated protease-cleaved (right) conformations. In an intact conformation, the PD-L1 Fab binding domain is exposed and able to bind their cognate target. The CD3 Fab domain is inhibited from binding by linkers in both the heavy and light chains that are both proteolytically cleavable and may be sequentially cleaved by matrix metalloproteases (MMPs) and/or cathepsins. A first cleavage event creates an intermediate active state which allows both PD-L1 and CD3 Fabs from a single protein construct to bind their cognate targets, thereby potentially directing killing of PD-L1+ cells by directed activation of CD3+ T cells. A second cleavage dissociates the PD-L1 and CD3 Fabs, removing the ability of a single molecule to bind both PD-L1 and CD3 simultaneously. FIG. 1B depicts a detailed diagram of an intact asymmetric LB (LockBody) protein consisting of heavy chain, light chain and Fc Stump. Each light chain is comprised of two Fabs linked by a lower hinge linker (LHL). Each heavy chain is comprised of two Fabs linked by a lower hinge linker (LHL) an Fc hinge and Fc fragment containing both CH2 and CH3 domains with either N-linked glycosylation sites (CH2) or Knobs mutations (CH3). Finally, the asymmetric LB protein contains a third polypeptide which contains both CH2 and CH3 domains with either N-linked glycosylation sites (CH2) or Holes mutations (CH3). FIG. 1C depicts diagrams of alternative formats for the protein molecules disclosed herein, where the molecule may; lack an Fc fragment (left); may lack an Fc Fragment but achieve half-life extension (HLE) through an alternative mechanism such as PEGylation, addition of an albumin binding domain, etc. (center); may contain an Fc fragment but be constructed from only two polypeptides (right) as symmetric one-arm constructs. Each polypeptide chain contains either two light chain Fabs linked by lower hinge linkers, an Fc hinge without disulfide bonds and Fc fragment (CH2 and CH3 domains) or two heavy chain Fabs linked by lower hinge linkers, an Fc hinge without disulfide bonds and Fc fragment (CH2 and CH3 domains). FIG. 1D depicts detailed diagrams of asymmetric one-arm constructs in which the two polypeptide chains contain one light chain and one heavy chain Fab linked by LHL. As above, each polypeptide chain further contains an Fc hinge without disulfide bonds and Fc fragment (CH2 and CH3 domains).

The first linker and the second linker are cleavable by matrix metalloproteases (MMPs) and/or cathepsins found in diseased tissues, such as tumors. The linkers in the protein are immunoglobulin-derived hinge sequences that are both proteolytically sensitive and may be sequentially cleaved, with a first cleavage taking the intact structure and creating an intermediate active state which allows an anti-PD-L1 Fab and an ant-CD3 Fab from a single protein construct to bind their cognate targets. A second cleavage event in the second linker removes the covalent linkage between the ant-PD-L1 Fab and the anti-CD3 Fab, abrogating the ability of the molecule to recruit T cell killing of PD-LT+ cells. This secondary cleavage event thereby constitutes a “self-destruct mechanism” that minimizes risk of activated molecule escaping the tumor microenvironment. Cleaved linkers based on immunoglobulin hinge sequences may also recruit increased immune effector function (antibody-dependent cellular cytotoxicity (ADCC), complement-dependent cytotoxicity (CDC) and antibody-dependent cellular phagocytosis (ADCP)) at the cell membrane via endogenous anti-hinge antibodies, which are a known phenomenon in human patients with (and even without) underlying autoreactive disease.

In some embodiments, a protein comprises one or more amino acid sequences provided in Table 1, Table 2, Table 6, Table 7, or Table 8.

TABLE 1
Exemplary sequences

| | | SEQ |
|---|---|---|
| Domain or Region | Sequence | ID NO |
| Linker LHL | GPAPELLGGGS | 1 |
| Linker LHL-F | GPAPLGLGGGS | 2 |
| Linker LHLX | GPAPELLGGGGS | 3 |
| Linker LHLX-F | GPAPLGLGGGGS | 4 |
| Linker LHLR-F | GPAPLGLRGGS | 5 |
| Linker LHLRX-F | GPAPLGLRGGGS | 6 |
| Linker L1 | GGGGS | 7 |
| Linker L2 | GGGGSGGGGS | 8 |
| Linker L3 | GGGGSGGGGSGGGGS | 9 |
| Linker LH | GPAPELL | 10 |
| Linker LHL-M | PPCPAPELLGGGS | 11 |
| Linker LHL-MF | PPCPAPLGLGGGS | 12 |
| Human CH1 domain | ASTKGPSVFPLAPSSKSTSGGTAALGCLVKDYFPE | 13 |
| | PVTVSWNSGALTSGVHTFPAVLQSSGLYSLSSVVT | |
| | VPSSSLGTQTYICNVNHKPSNTKVDKKVEPKSC | |
| Human CH1-hinge- | ASTKGPSVFPLAPSSKSTSGGTAALGCLVKDYFPE | 14 |
| CH2-CH3 sequence | PVTVSWNSGALTSGVHTFPAVLQSSGLYSLSSVVT | |
| (IgG1 wild type) | VPSSSLGTQTYICNVNHKPSNTKVDKKVEPKSCDK | |
| | THTCPPCPAPELLGGPSVFLFPPKPKDTLMISRTP | |
| | EVTCVVVDVSHEDPEVKFNWYVDGVEVHNAKTKPR | |
| | EEQYNSTYRVVSVLTVLHQDWLNGKEYKCKVSNKA | |
| | LPAPIEKTISKAKGQPREPQVYTLPPSRDELTKNQ | |
| | VSLTCLVKGFYPSDIAVEWESNGQPENNYKTTPPV | |
| | LDSDGSFFLYSKLTVDKSRWQQGNVFSCSVMHEAL | |
| | HNHYTQKSLSLSPGK | |
| Human Cₖₐₚₚₐ | RTVAAPSVFIFPPSDEQLKSGTASVVCLLNNFYPR | 15 |
| domain (Ck) | EAKVQWKVDNALQSGNSQESVTEQDSKDSTYSLSS | |
| | TLTLSKADYEKHKVYACEVTHQGLSSPVTKSFNRG | |
| | EC | |
| Human IgG1-3M | ASTKGPSVFPLAPSSKSTSGGTAALGCLVKDYFPE | 16 |
| | PVTVSWNSGALTSGVHTFPAVLQSSGLYSLSSVVT | |
| | VPSSSLGTQTYICNVNHKPSNTKVDKKVEPKSCDK | |
| | THTCPPCPAPEAAGAPSVFLFPPKPKDTLMISRTP | |
| | EVTCVVVDVSHEDPEVKFNWYVDGVEVHNAKTKPR | |
| | EEQYNSTYRVVSVLTVLHQDWLNGKEYKCKVSNKA | |
| | LPAPIEKTISKAKGQPREPQVYTLPPSRDELTKNQ | |
| | VSLTCLVKGFYPSDIAVEWESNGQPENNYKTTPPV | |
| | LDSDGSFFLYSKLTVDKSRWQQGNVFSCSVMHEAL | |
| | HNHYTQKSLSLSPGK | |
| Human IgG2 wild | ASTKGPSVFPLAPCSRSTSESTAALGCLVKDYFPE | 17 |
| type | PVTVSWNSGALTSGVHTFPAVLQSSGLYSLSSVVT | |
| | VPSSNFGTQTYTCNVDHKPSNTKVDKTVERKCCVE | |
| | CPPCPAPPVAGPSVFLFPPKPKDTLMISRTPEVTC | |
| | VVVDVSHEDPEVQFNWYVDGVEVHNAKTKPREEQF | |
| | NSTFRVVSVLTVVHQDWLNGKEYKCKVSNKGLPAP | |
| | IEKTISKTKGQPREPQVYTLPPSREEMTKNQVSLT | |
| | CLVKGFYPSDISVEWESNGQPENNYKTTPPMLDSD | |
| | GSFFLYSKLTVDKSRWQQGNVFSCSVMHEALHNHY | |
| | TQKSLSLSPGK | |
| Human IgG4 wild | ASTKGPSVFPLAPCSRSTSESTAALGCLVKDYFPE | 18 |
| type | PVTVSWNSGALTSGVHTFPAVLQSSGLYSLSSVVT | |
| | VPSSSLGTKTYTCNVDHKPSNTKVDKRVESKYGPP | |
| | CPSCPAPEFLGGPSVFLFPPKPKDTLMISRTPEVT | |
| | CVVVDVSQED PEVQFNWYVDGVEVHNAKTKPREE | |
| | QFNSTYRVVSVLTVLHQDWLNGKEYKCKVSNKGLP | |
| | SSIEKTISKAKGQPREPQVYTLPPSQEEMTKNQVS | |
| | LTCLVKGFYPSDIAVEWESNGQPENNYKTTPPVLD | |
| | SDGSFFLYSRLTVDKSRWQEGNVFSCSVMHEALHN | |
| | HYTQKSLSLSLGK | |
| Human IgG4 | ASTKGPSVFPLAPCSRSTSESTAALGCLVKDYFPE | 19 |
| (S228P) | PVTVSWNSGALTSGVHTFPAVLQSSGLYSLSSVVT | |
| | VPSSSLGTKTYTCNVDHKPSNTKVDKRVESKYGPP | |
| | CPPCPAPEFLGGPSVFLFPPKPKDTLMISRTPEVT | |
| | CVVVDVSQED PEVQFNWYVDGVEVHNAKTKPREE | |
| | QFNSTYRVVSVLTVLHQDWLNGKEYKCKVSNKGLP | |
| | SSIEKTISKAKGQPREPQVYTLPPSQEEMTKNQVS | |
| | LTCLVKGFYPSDIAVEWESNGQPENNYKTTPPVLD | |
| | SDGSFFLYSRLTVDKSRWQEGNVFSCSVMHEALHN | |
| | HYTQKSLSLSLGK | |
| Anti-PD-L1 HCDR1 | TYAIS | 20 |
| Anti-PD-L1 HCDR2 | GIIPIFGKAHYAQKFQG | 21 |
| Anti-PD-L1 HCDR3 | KFHFVSGSPFGMDV | 22 |
| Anti-PD-L1 LCDR1 | RASQSVSSYLA | 23 |
| Anti-PD-L1 LCDR2 | DASNRAT | 24 |
| Anti-PD-L1 LCDR3 | QQRSNWPT | 25 |
| Anti-CD3 HCDR1 | TYAMN | 26 |
| Anti-CD3 HCDR2 | RIRSKYNNYATYYADSVKD | 27 |
| Anti-CD3 HCDR2 | RIRSKYNNYATYYADSVKG | 28 |
| Anti-CD3 HCDR3 | HGNFGNSYVSWFAY | 29 |
| Anti-CD3 LCDR1 | RSSTGAVTTSNYAN | 30 |
| Anti-CD3 LCDR2 | GTNKRAP | 31 |
| Anti-CD3 LCDR3 | ALWYSNLWV | 32 |
| Anti-PD-L1 VH | QVQLVQSGAEVKKPGSSVKVSCKTSGDTFSTYAIS | 33 |
| domain | WVRQAPGQGLEWMGGIIPIFGKAHYAQKFQGRVTI | |
| | TADESTSTAYMELSSLRSEDTAVYFCARKFHFVSG | |
| | SPFGMDVWGQGTTVTVSS | |
| Anti-PD-L1 VL | EIVLTQSPATLSLSPGERATLSCRASQSVSSYLAW | 34 |
| domain | YQQKPGQAPRLLIYDASNRATGIPARFSGSGSGTD | |
| | FTLTISSLEPEDFAVYYCQQRSNWPTFGQGTKVEI | |
| | K | |

In the VH and VL domain sequences, the CDR sequences are underlined.

TABLE 2
Anti-CD3 SP34 Fv humanization variants and murine sequences

| | SEQ | |
|---|---|---|
| Name | ID NO | Fv Sequence |
| 1LC | 37 | QAVVTQEPSLTVSPGGTVTLTCRSSTGAVTTSNYANWVQQKPDH |
| | | LPRALIGGTNKRAPGTPARFSGSLLGDKAALTLSGVQPEDEAEY |
| | | YCALWYSNLWVFGGGTKLTVL |
| 2LC | 38 | QTVVTQEPSLTVSPGGTVTLTCRSSTGAVTTSNYPNWVQQKPGQ |
| | | APRALIGGTNKRAPGTPARFSGSLLGGKAALTLSGVQPEDEAEY |
| | | YCALWYSNLWVFGGGTKLTVL |
| 3LC | 39 | QAVVTQEPSLTVSPGGTVTLTCRSSTGAVTTSNYANWVQQKPDH |
| | | LPRALIGGTNKRAPGVPARFSGSLLGDKAALTISGAQPEDEAIY |
| | | YCALWYSNLWVFGGGTKLTVL |
| 4LC | 40 | QAVVTQEPSLTVSPGGTVTLTCRSSTGAVTSGYYANWVQQKPDH |
| | | LPRALIGGTNKRAPGTPARFSGSLLGDKAALTLSGVQPEDEAEY |
| | | YCALWYSNLWVFGGGTKLTVL |
| 5LC | 41 | QTVVTQEPSLTVSPGGTVTLTCRSSTGAVTSGYYPNWVQQKPGQ |
| | | APRALIGGTNKRAPGTPARFSGSLLGGKAALTLSGVQPEDEAEY |
| | | YCALWYSNLWVFGGGTKLTVL |
| 6LC | 42 | QTVVTQEPSLTVSPGGTVTLTCGSSTGAVTSGYYPNWVQQKPGQ |
| | | APRGLIGGTKFLAPGTPARFSGSLLGGKAALTLSGVQPEDEAEY |
| | | YCALWYSNRWVFGGGTKLTVL |
| 7LC = murine | 43 | QAVVTQESALTTSPGETVTLTCRSSTGAVTTSNYANWVQEKPDH |
| SP34_VL_Fv | | LFTGLIGGTNKRAPGVPARFSGSLIGDKAALTITGAQTEDEAIY |
| | | FCALWYSNLWVFGGGTKLTVL |
| 1HC | 44 | EVQLVESGGGLVQPGGSLRLSCAASGFTFNTYAMNWVRQAPGKG |
| | | LEWVGRIRSKYNNYATYYADSVKDRFTISRDDSQSILYLQMNSL |
| | | KTEDTAVYYCVRHGNFGNSYVSWFAYWGQGTLVTVSS |
| 2HC | 45 | EVQLVESGGGLVQPGGSLRLSCAASGFTFNTYAMNWVRQAPGKG |
| | | LEWVARIRSKYNNYATYYADSVKDRFTISRDDSQSILYLQMNSL |
| | | KTEDTAVYYCVRHGNFGNSYVSWFAYWGQGTLVTVSS |
| 3HC | 46 | EVQLVESGGGLVQPGGSLRLSCAASGFTFNTYAMNWVRQAPGKG |
| | | LEWVGRIRSKYNNYATYYADSVKGRFTISRDDSKNSLYLQMNSL |
| | | KTEDTAVYYCARHGNFGNSYVSWFAYWGQGTLVTVSS |
| 4HC | 47 | EVQLVESGGGLVQPGGSLRLSCAASGFTFSTYAMNWVRQAPGKG |
| | | LEWVGRIRSKYNNYATYYADSVKGRFTISRDDSKNSLYLQMNSL |
| | | KTEDTAVYYCARHGNFGNSYVSWFAYWGQGTLVTVSS |
| 5HC | 48 | EVQLVESGGGLVQPGGSLKLSCAASGFTFNKYAMNWVRQAPGKG |
| | | LEWVARIRSKYNNYATYYADSVKDRFTISRDDSKNTAYLQMNNL |
| | | KTEDTAVYYCVRHGNFGNSYISYWAYWGQGTLVTVSS |
| 6HC = Murine | 49 | EVQLVESGGGLVQPKGSLKLSCAASGFTFNTYAMNWVRQAPGKG |
| SP34_VH_Fv | | LEWVARIRSKYNNYATYYADSVKDRFTISRDDSQSILYLQMNNL |
| | | KTEDTAMYYCVRHGNFGNSYVSWFAYWGQGTLVTVSS |

Bold/underlined = humanized residue.
6L and 5H sequences are provided in WO 2014/167022.
LC = light chain
HC = heavy chain

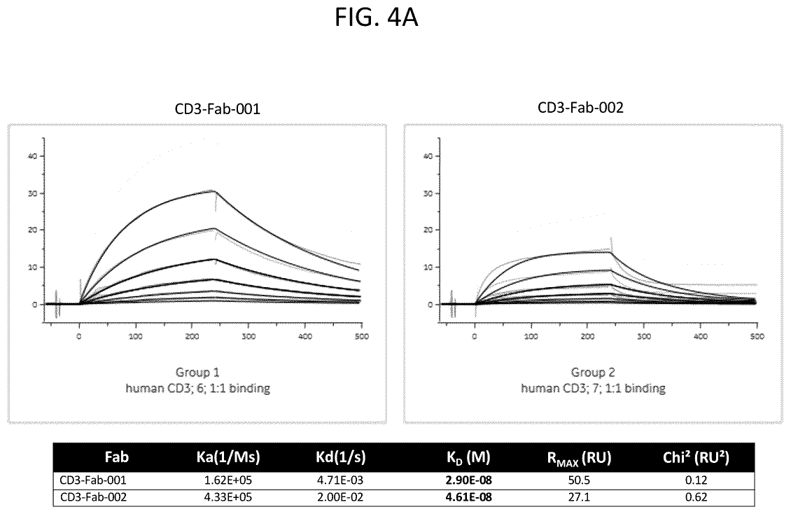

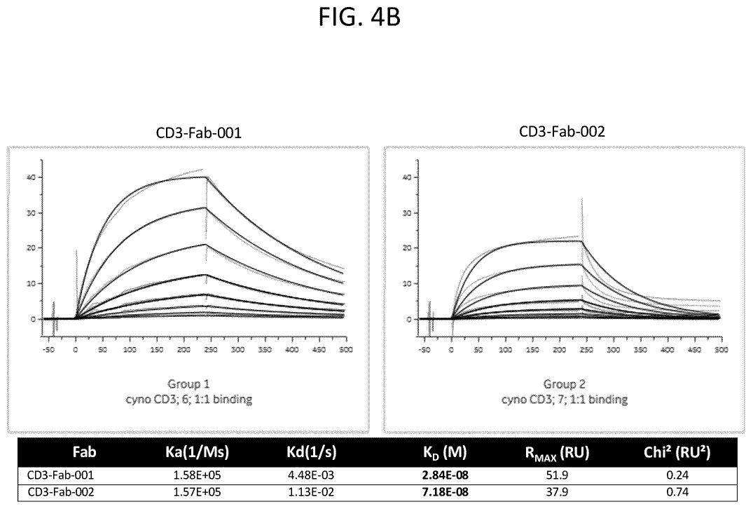

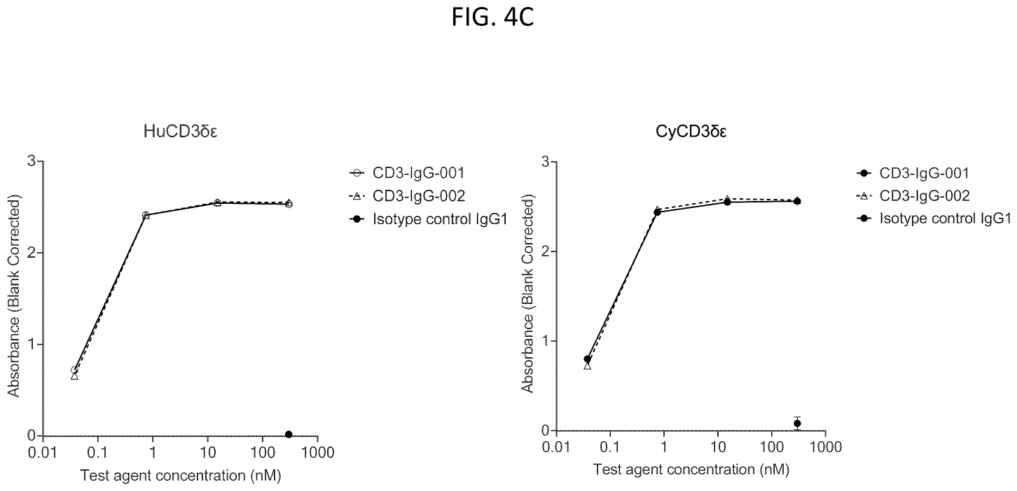

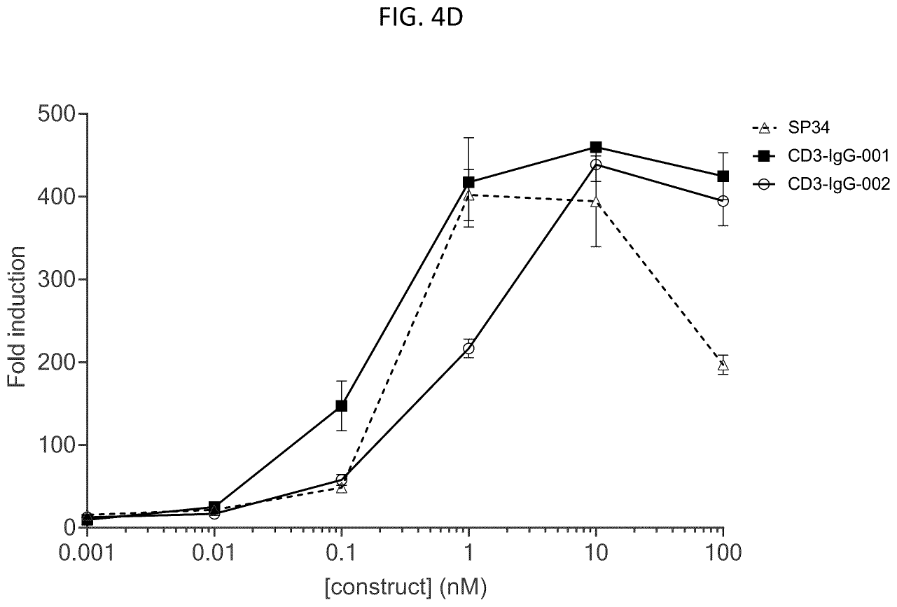

Table 3 describes selected Fab proteins generated with selected sequences from Table 2 as described. Both anti-CD3 Fab proteins were characterized further in SPR binding experiments as described in FIG. 4A.

TABLE 3
Chain sequence combinations of illustrative
anti-CD3 Fab sequences

| Name | Heavy Chain | Light Chain |
|---|---|---|
| Anti-CD3 Fab-001 | 2HC | 1LC |
| Anti-CD3 Fab-002 | 3HC | 3LC |

Table 4 describes selected IgG proteins generated with selected sequences from Table 2 as described. Both CD3 IgG proteins were characterized further in ELISA binding experiments and Jurkat reporter assay as described in FIG. 4B and FIG. 4C.

TABLE 4
Chain sequence combinations of illustrative anti-CD3 IgG sequences

| Name | Heavy Chain | Light Chain | Fc |
|---|---|---|---|
| CD3-IgG-001 | 2HC | 1LC | Human IgG1-3M |
| CD3-IgG-002 | 3HC | 3LC | Human IgG1-3M |

Table 5 describes anti-PD-L1 VH variants. Individual point mutations of CDRs are generated by in silico modelling and summarized in Table 5. The aim of these anti-PD-L1 variants was affinity modification to cynomolgus and/or human PD-L1.

TABLE 5
Anti-PD-L1 VH variants for affinity
modification generated by in silico modeling

| | | |
|---|---|---|
| Anti-PD-L1 | X₁YX₂IS | SEQ ID NO: 50 |
| HCDR1 | | |
| Anti-PD-L1 | X₃IX₄PX₅FX₆X₇AX₈YA | SEQ ID NO: 51 |
| HCDR2 | X₉KFQG | |
| Anti-PD-L1 | X₁₀KFHFVX₁₁X₁₂X₁₃PF | SEQ ID NO: 52 |
| HCDR3 | GMDV | |

X₁ = T, R, Q
X₂ = A, S, H, L
X₃ = G, N, H
X₄ = I, F
X₅ = I, K, W, L, Y, Q, R
X₆ = G, H, Q, N
X₇ = K, I, F, M, D, Y, L, W, H, R
X₈ = H, L
X₉ = Q, E
X₁₀ = R, K, F, N, H, P, Q
X₁₁ = S, W, R, L, Q, G, Y, D, N, A, M
X₁₂ = G, A, V, S, Q, N
X₁₃ = S, P

Table 6 depicts selected anti-PD-L1 VH variants heavy domains based on Table 5. The aim of these anti-PD-L1 variants was affinity maturation to cynomolgus and/or human PD-L1. Expression of each of the VH variable domains with wild-type (WT) VL variable domains in Fab format was carried out as well as subsequent determination of KD to human and cynomolgus PD-L1 in SPR.

TABLE 6
Selected anti-PD-L1 VH variants based
on VH CDR mutations outlined in Table 5.

| Name | Mutation | Sequence |
|---|---|---|
| WT VH | | QVQLVQSGAEVKKPGSSVKVSCKTSGDTFSTYAISWVRQAPGQG |
| PD-L1 | | LEWMGGIIPIFGKAHYAQKFQGRVTITADESTSTAYMELSSLRS |
| (SEQ ID | | EDTAVYFCARKFHFVSGSPFGMDVWGQGTTVTVSS |
| NO: 33) | | |
| mut-01 | G105N | QVQLVQSGAEVKKPGSSVKVSCKTSGDTFSTYAISWVRQAPGQG |
| (SEQ ID | | LEWMGGIIPIFGKAHYAQKFQGRVTITADESTSTAYMELSSLRS |
| NO: 54) | | EDTAVYFCARKFHFVSNSPFGMDVWGQGTTVTVSS |
| mut-02 | G105Q | QVQLVQSGAEVKKPGSSVKVSCKTSGDTFSTYAISWVRQAPGQG |
| (SEQ ID | | LEWMGGIIPIFGKAHYAQKFQGRVTITADESTSTAYMELSSLRS |
| NO: 55) | | EDTAVYFCARKFHFVSQSPFGMDVWGQGTTVTVSS |
| mut-03 | S104W | QVQLVQSGAEVKKPGSSVKVSCKTSGDTFSTYAISWVRQAPGQG |
| (SEQ ID | | LEWMGGIIPIFGKAHYAQKFQGRVTITADESTSTAYMELSSLRS |
| NO: 56) | | EDTAVYFCARKFHFVWGSPFGMDVWGQGTTVTVSS |
| mut-04 | I54K | QVQLVQSGAEVKKPGSSVKVSCKTSGDTFSTYAISWVRQAPGQG |
| (SEQ ID | | LEWMGGIIPKFGKAHYAQKFQGRVTITADESTSTAYMELSSLRS |
| NO: 57) | | EDTAVYFCARKFHFVSGSPFGMDVWGQGTTVTVSS |
| mut-05 | G105S | QVQLVQSGAEVKKPGSSVKVSCKTSGDTFSTYAISWVRQAPGQG |
| (SEQ ID | | LEWMGGIIPIFGKAHYAQKFQGRVTITADESTSTAYMELSSLRS |
| NO: 58) | | EDTAVYFCARKFHFVSSSPFGMDVWGQGTTVTVSS |
| mut-06 | S104R | QVQLVQSGAEVKKPGSSVKVSCKTSGDTFSTYAISWVRQAPGQG |
| (SEQ ID | | LEWMGGIIPIFGKAHYAQKFQGRVTITADESTSTAYMELSSLRS |
| NO: 59) | | EDTAVYFCARKFHFVRGSPFGMDVWGQGTTVTVSS |
| mut-07 | I54W | QVQLVQSGAEVKKPGSSVKVSCKTSGDTFSTYAISWVRQAPGQG |
| (SEQ ID | | LEWMGGIIPWFGKAHYAQKFQGRVTITADESTSTAYMELSSLRS |
| NO: 60) | | EDTAVYFCARKFHFVSGSPFGMDVWGQGTTVTVSS |
| mut-08 | K57R | QVQLVQSGAEVKKPGSSVKVSCKTSGDTFSTYAISWVRQAPGQG |
| (SEQ ID | | LEWMGGIIPIFGRAHYAQKFQGRVTITADESTSTAYMELSSLRS |
| NO: 61) | | EDTAVYFCARKFHFVSGSPFGMDVWGQGTTVTVSS |
| mut-09 | S104L | QVQLVQSGAEVKKPGSSVKVSCKTSGDTFSTYAISWVRQAPGQG |
| (SEQ ID | | LEWMGGIIPIFGKAHYAQKFQGRVTITADESTSTAYMELSSLRS |
| NO: 62) | | EDTAVYFCARKFHFVLGSPFGMDVWGQGTTVTVSS |
| mut-10 | S104Q | QVQLVQSGAEVKKPGSSVKVSCKTSGDTFSTYAISWVRQAPGQG |
| (SEQ ID | | LEWMGGIIPIFGKAHYAQKFQGRVTITADESTSTAYMELSSLRS |
| NO: 63) | | EDTAVYFCARKFHFVQGSPFGMDVWGQGTTVTVSS |
| mut-11 | S104G | QVQLVQSGAEVKKPGSSVKVSCKTSGDTFSTYAISWVRQAPGQG |
| (SEQ ID | | LEWMGGIIPIFGKAHYAQKFQGRVTITADESTSTAYMELSSLRS |
| NO: 64) | | EDTAVYFCARKFHFVGGSPFGMDVWGQGTTVTVSS |
| mut-12 | S104Y | QVQLVQSGAEVKKPGSSVKVSCKTSGDTFSTYAISWVRQAPGQG |
| (SEQ ID | | LEWMGGIIPIFGKAHYAQKFQGRVTITADESTSTAYMELSSLRS |
| NO: 65) | | EDTAVYFCARKFHFVYGSPFGMDVWGQGTTVTVSS |
| mut-13 | K57H | QVQLVQSGAEVKKPGSSVKVSCKTSGDTFSTYAISWVRQAPGQG |
| (SEQ ID | | LEWMGGIIPIFGHAHYAQKFQGRVTITADESTSTAYMELSSLRS |
| NO: 66) | | EDTAVYFCARKFHFVSGSPFGMDVWGQGTTVTVSS |
| mut-14 | S104D | QVQLVQSGAEVKKPGSSVKVSCKTSGDTFSTYAISWVRQAPGQG |
| (SEQ ID | | LEWMGGIIPIFGKAHYAQKFQGRVTITADESTSTAYMELSSLRS |
| NO: 67) | | EDTAVYFCARKFHFVDGSPFGMDVWGQGTTVTVSS |
| mut-15 | K57W | QVQLVQSGAEVKKPGSSVKVSCKTSGDTFSTYAISWVRQAPGQG |
| (SEQ ID | | LEWMGGIIPIFGWAHYAQKFQGRVTITADESTSTAYMELSSLRS |
| NO: 68) | | EDTAVYFCARKFHFVSGSPFGMDVWGQGTTVTVSS |
| mut-16 | G105V | QVQLVQSGAEVKKPGSSVKVSCKTSGDTFSTYAISWVRQAPGQG |
| (SEQ ID | | LEWMGGIIPIFGKAHYAQKFQGRVTITADESTSTAYMELSSLRS |
| NO: 69) | | EDTAVYFCARKFHFVSVSPFGMDVWGQGTTVTVSS |
| mut-17 | I54L | QVQLVQSGAEVKKPGSSVKVSCKTSGDTFSTYAISWVRQAPGQG |
| (SEQ ID | | LEWMGGIIPLFGKAHYAQKFQGRVTITADESTSTAYMELSSLRS |
| NO: 70) | | EDTAVYFCARKFHFVSGSPFGMDVWGQGTTVTVSS |
| mut-18 | K57L | QVQLVQSGAEVKKPGSSVKVSCKTSGDTFSTYAISWVRQAPGQG |
| (SEQ ID | | LEWMGGIIPIFGLAHYAQKFQGRVTITADESTSTAYMELSSLRS |
| NO: 71) | | EDTAVYFCARKFHFVSGSPFGMDVWGQGTTVTVSS |
| mut-19 | A33L | QVQLVQSGAEVKKPGSSVKVSCKTSGDTFSTYLISWVRQAPGQG |
| (SEQ ID | | LEWMGGIIPIFGKAHYAQKFQGRVTITADESTSTAYMELSSLRS |
| NO: 72) | | EDTAVYFCARKFHFVSGSPFGMDVWGQGTTVTVSS |
| mut-20 | I54Y | QVQLVQSGAEVKKPGSSVKVSCKTSGDTFSTYAISWVRQAPGQG |
| (SEQ ID | | LEWMGGIIPYFGKAHYAQKFQGRVTITADESTSTAYMELSSLRS |
| NO: 73) | | EDTAVYFCARKFHFVSGSPFGMDVWGQGTTVTVSS |
| mut-21 | G105A | QVQLVQSGAEVKKPGSSVKVSCKTSGDTFSTYAISWVRQAPGQG |
| (SEQ ID | | LEWMGGIIPIFGKAHYAQKFQGRVTITADESTSTAYMELSSLRS |
| NO: 74) | | EDTAVYFCARKFHFVSASPFGMDVWGQGTTVTVSS |
| mut-22 | A33H | QVQLVQSGAEVKKPGSSVKVSCKTSGDTFSTYHISWVRQAPGQG |
| (SEQ ID | | LEWMGGIIPIFGKAHYAQKFQGRVTITADESTSTAYMELSSLRS |
| NO: 75) | | EDTAVYFCARKFHFVSGSPFGMDVWGQGTTVTVSS |
| mut-23 | G56H | QVQLVQSGAEVKKPGSSVKVSCKTSGDTFSTYAISWVRQAPGQG |
| (SEQ ID | | LEWMGGIIPIFHKAHYAQKFQGRVTITADESTSTAYMELSSLRS |
| NO: 76) | | EDTAVYFCARKFHFVSGSPFGMDVWGQGTTVTVSS |
| mut-24 | K57Y | QVQLVQSGAEVKKPGSSVKVSCKTSGDTFSTYAISWVRQAPGQG |
| (SEQ ID | | LEWMGGIIPIFGYAHYAQKFQGRVTITADESTSTAYMELSSLRS |
| NO: 77) | | EDTAVYFCARKFHFVSGSPFGMDVWGQGTTVTVSS |
| mut-25 | R98Q | QVQLVQSGAEVKKPGSSVKVSCKTSGDTFSTYAISWVRQAPGQG |
| (SEQ ID | | LEWMGGIIPIFGKAHYAQKFQGRVTITADESTSTAYMELSSLRS |
| NO: 78) | | EDTAVYFCAQKFHFVSGSPFGMDVWGQGTTVTVSS |
| mut-26 | R98P | QVQLVQSGAEVKKPGSSVKVSCKTSGDTFSTYAISWVRQAPGQG |
| (SEQ ID | | LEWMGGIIPIFGKAHYAQKFQGRVTITADESTSTAYMELSSLRS |
| NO: 79) | | EDTAVYFCAPKFHFVSGSPFGMDVWGQGTTVTVSS |
| mut-27 | K57D | QVQLVQSGAEVKKPGSSVKVSCKTSGDTFSTYAISWVRQAPGQG |
| (SEQ ID | | LEWMGGIIPIFGDAHYAQKFQGRVTITADESTSTAYMELSSLRS |
| NO: 80) | | EDTAVYFCARKFHFVSGSPFGMDVWGQGTTVTVSS |
| mut-28 | S104N | QVQLVQSGAEVKKPGSSVKVSCKTSGDTFSTYAISWVRQAPGQG |
| (SEQ ID | | LEWMGGIIPIFGKAHYAQKFQGRVTITADESTSTAYMELSSLRS |
| NO: 81) | | EDTAVYFCARKFHFVNGSPFGMDVWGQGTTVTVSS |
| mut-29 | R98H | QVQLVQSGAEVKKPGSSVKVSCKTSGDTFSTYAISWVRQAPGQG |
| (SEQ ID | | LEWMGGIIPIFGKAHYAQKFQGRVTITADESTSTAYMELSSLRS |
| NO: 82) | | EDTAVYFCAHKFHFVSGSPFGMDVWGQGTTVTVSS |
| mut-30 | R98N | QVQLVQSGAEVKKPGSSVKVSCKTSGDTFSTYAISWVRQAPGQG |
| (SEQ ID | | LEWMGGIIPIFGKAHYAQKFQGRVTITADESTSTAYMELSSLRS |
| NO: 83) | | EDTAVYFCANKFHFVSGSPFGMDVWGQGTTVTVSS |
| mut-31 | G50H | QVQLVQSGAEVKKPGSSVKVSCKTSGDTFSTYAISWVRQAPGQG |
| (SEQ ID | | LEWMGHIIPIFGKAHYAQKFQGRVTITADESTSTAYMELSSLRS |
| NO: 84) | | EDTAVYFCARKFHFVSGSPFGMDVWGQGTTVTVSS |
| mut-32 | S104A | QVQLVQSGAEVKKPGSSVKVSCKTSGDTFSTYAISWVRQAPGQG |
| (SEQ ID | | LEWMGGIIPIFGKAHYAQKFQGRVTITADESTSTAYMELSSLRS |
| NO: 85) | | EDTAVYFCARKFHFVAGSPFGMDVWGQGTTVTVSS |
| mut-33 | A33S | QVQLVQSGAEVKKPGSSVKVSCKTSGDTFSTYSISWVRQAPGQG |
| (SEQ ID | | LEWMGGIIPIFGKAHYAQKFQGRVTITADESTSTAYMELSSLRS |
| NO: 86) | | EDTAVYFCARKFHFVAGSPFGMDVWGQGTTVTVSS |
| mut-34 | K57M | QVQLVQSGAEVKKPGSSVKVSCKTSGDTFSTYAISWVRQAPGQG |
| (SEQ ID | | LEWMGGIIPIFGMAHYAQKFQGRVTITADESTSTAYMELSSLRS |
| NO: 87) | | EDTAVYFCARKFHFVSGSPFGMDVWGQGTTVTVSS |
| mut-35 | S104M | QVQLVQSGAEVKKPGSSVKVSCKTSGDTFSTYAISWVRQAPGQG |
| (SEQ ID | | LEWMGGIIPIFGKAHYAQKFQGRVTITADESTSTAYMELSSLRS |
| NO: 88) | | EDTAVYFCARKFHFVMGSPFGMDVWGQGTTVTVSS |
| mut-36 | G50N | QVQLVQSGAEVKKPGSSVKVSCKTSGDTFSTYAISWVRQAPGQG |
| (SEQ ID | | LEWMGNIIPIFGKAHYAQKFQGRVTITADESTSTAYMELSSLRS |
| NO: 89) | | EDTAVYFCARKFHFVSGSPFGMDVWGQGTTVTVSS |
| mut-37 | R98F | QVQLVQSGAEVKKPGSSVKVSCKTSGDTFSTYAISWVRQAPGQG |
| (SEQ ID | | LEWMGGIIPIFGKAHYAQKFQGRVTITADESTSTAYMELSSLRS |
| NO: 90) | | EDTAVYFCAFKFHFVSGSPFGMDVWGQGTTVTVSS |
| mut-38 | R98K | QVQLVQSGAEVKKPGSSVKVSCKTSGDTFSTYAISWVRQAPGQG |
| (SEQ ID | | LEWMGGIIPIFGKAHYAQKFQGRVTITADESTSTAYMELSSLRS |
| NO: 91) | | EDTAVYFCAKKFHFVSGSPFGMDVWGQGTTVTVSS |
| mut-39 | K57F | QVQLVQSGAEVKKPGSSVKVSCKTSGDTFSTYAISWVRQAPGQG |
| (SEQ ID | | LEWMGGIIPIFGFAHYAQKFQGRVTITADESTSTAYMELSSLRS |
| NO: 92) | | EDTAVYFCARKFHFVSGSPFGMDVWGQGTTVTVSS |
| mut-40 | K57I | QVQLVQSGAEVKKPGSSVKVSCKTSGDTFSTYAISWVRQAPGQG |
| (SEQ ID | | LEWMGGIIPIFGIAHYAQKFQGRVTITADESTSTAYMELSSLRS |
| NO: 93) | | EDTAVYFCARKFHFVSGSPFGMDVWGQGTTVTVSS |
| mut-41 | S106P | QVQLVQSGAEVKKPGSSVKVSCKTSGDTFSTYAISWVRQAPGQG |
| (SEQ ID | | LEWMGGIIPIFGKAHYAQKFQGRVTITADESTSTAYMELSSLRS |
| NO: 94) | | EDTAVYFCARKFHFVSGPPFGMDVWGQGTTVTVSS |
| mut-42 | I54Q | QVQLVQSGAEVKKPGSSVKVSCKTSGDTFSTYAISWVRQAPGQG |
| (SEQ ID | | LEWMGGIIPQFGKAHYAQKFQGRVTITADESTSTAYMELSSLRS |
| NO: 95) | | EDTAVYFCARKFHFVSGSPFGMDVWGQGTTVTVSS |
| mut-43 | G56Q | QVQLVQSGAEVKKPGSSVKVSCKTSGDTFSTYAISWVRQAPGQG |
| (SEQ ID | | LEWMGGIIPIFQKAHYAQKFQGRVTITADESTSTAYMELSSLRS |
| NO: 96) | | EDTAVYFCARKFHFVSGSPFGMDVWGQGTTVTVSS |
| mut-44 | I52F | QVQLVQSGAEVKKPGSSVKVSCKTSGDTFSTYAISWVRQAPGQG |
| (SEQ ID | | LEWMGGIFPIFGKAHYAQKFQGRVTITADESTSTAYMELSSLRS |
| NO: 97) | | EDTAVYFCARKFHFVSGSPFGMDVWGQGTTVTVSS |
| mut-45 | H59L | QVQLVQSGAEVKKPGSSVKVSCKTSGDTFSTYAISWVRQAPGQG |
| (SEQ ID | | LEWMGGIIPIFGKALYAQKFQGRVTITADESTSTAYMELSSLRS |
| NO: 98) | | EDTAVYFCARKFHFVSGSPFGMDVWGQGTTVTVSS |
| mut-46 | I54R | QVQLVQSGAEVKKPGSSVKVSCKTSGDTFSTYAISWVRQAPGQG |
| (SEQ ID | | LEWMGGIIPRFGKAHYAQKFQGRVTITADESTSTAYMELSSLRS |
| NO: 99) | | EDTAVYFCARKFHFVSGSPFGMDVWGQGTTVTVSS |
| mut-47 | T31R | QVQLVQSGAEVKKPGSSVKVSCKTSGDTFSRYAISWVRQAPGQG |
| (SEQ ID | | LEWMGGIIPIFGKAHYAQKFQGRVTITADESTSTAYMELSSLRS |
| NO: 100) | | EDTAVYFCARKFHFVSGSPFGMDVWGQGTTVTVSS |
| mut-48 | T31Q | QVQLVQSGAEVKKPGSSVKVSCKTSGDTFSQYAISWVRQAPGQG |
| (SEQ ID | | LEWMGGIIPIFGKAHYAQKFQGRVTITADESTSTAYMELSSLRS |
| NO: 101) | | EDTAVYFCARKFHFVSGSPFGMDVWGQGTTVTVSS |
| mut-49 | G56N | QVQLVQSGAEVKKPGSSVKVSCKTSGDTFSTYAISWVRQAPGQG |
| (SEQ ID | | LEWMGGIIPIFNKAHYAQKFQGRVTITADESTSTAYMELSSLRS |
| NO: 102) | | EDTAVYFCARKFHFVSGSPFGMDVWGQGTTVTVSS |
| mut-50 | Q62E | QVQLVQSGAEVKKPGSSVKVSCKTSGDTFSTYAISWVRQAPGQG |
| (SEQ ID | | LEWMGGIIPIFGKAHYAEKFQGRVTITADESTSTAYMELSSLRS |
| NO: 103) | | EDTAVYFCARKFHFVSGSPFGMDVWGQGTTVTVSS |
| mut-51 | S104R, | QVQLVQSGAEVKKPGSSVKVSCKTSGDTFSQYAISWVRQAPGQG |
| (SEQ ID | G56Q, | LEWMGGIIPIFQKAHYAQKFQGRVTITADESTSTAYMELSSLRS |
| NO: 104) | T31Q | EDTAVYFCARKFHFVRGSPFGMDVWGQGTTVTVSS |
| mut-52 | S104R, | QVQLVQSGAEVKKPGSSVKVSCKTSGDTFSTYAISWVRQAPGQG |
| (SEQ ID | G56Q | LEWMGGIIPIFQKAHYAQKFQGRVTITADESTSTAYMELSSLRS |
| NO: 105) | | EDTAVYFCARKFHFVRGSPFGMDVWGQGTTVTVSS |
| mut-53 | S104R, | QVQLVQSGAEVKKPGSSVKVSCKTSGDTFSQYAISWVRQAPGQG |
| (SEQ ID | T31Q | LEWMGGIIPIFGKAHYAQKFQGRVTITADESTSTAYMELSSLRS |
| NO: 106) | | EDTAVYFCARKFHFVRGSPFGMDVWGQGTTVTVSS |
| mut-54 | G56Q, | QVQLVQSGAEVKKPGSSVKVSCKTSGDTFSQYAISWVRQAPGQG |
| (SEQ ID | T31Q | LEWMGGIIPIFQKAHYAQKFQGRVTITADESTSTAYMELSSLRS |
| NO: 107) | | EDTAVYFCARKFHFVSGSPFGMDVWGQGTTVTVSS |

Table 7 depicts illustrative full-length LB protein light and heavy chain sequences. Each chain contains WT anti-PD-L1 variable domains followed by LHL and CD3 Fab variant. For asymmetric one-arm constructs, heavy chains, Fe hinge and relevant Fe fragments are present as described in FIG. TA and FIG. TB (right). For constructs described in FIG. TB (left, center) each chain contains WT anti-PD-L1 variable domains followed by LHL and CD3 Fab variant. For symmetric one-arm constructs, both light and heavy chain sequences are followed by Fc hinge and Fc fragment as illustrated in FIG. TC. Each polypeptide chain in Table 7 is named with: first, a number from 1-7 referencing the respective CD3 humanization chain used (Table 2); second, by either HH or LL or B-HH or B-LL referring to asymmetric or symmetric constructs (B-); and third, an optional letter or letters from X, F, R-F or RX-F, which denote details on the LHL sequence as specified in Table 1. Finally, an addition of HLE in this table refers to the option of adding an appropriate half-life extension moiety.

TABLE 7
Illustrative full-length light chain (LL)
sequences and heavy (HH) chain sequences

| | SEQ ID | |
|---|---|---|
| Name | NO | Amino Acid sequence |
| 1LL-F | 108 | EIVLTQSPATLSLSPGERATLSCRASQSVSSYLAWYQQKPGQAPRLLIYD |
| | | ASNRATGIPARFSGSGSGTDFTLTISSLEPEDFAVYYCQQRSNWPTFGQG |
| | | TKVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGL |
| | | SSPVTKSFNRGECGPAPLGLGGGSQAVVTQEPSLTVSPGGTVTLTCRSST |
| | | GAVTTSNYANWVQQKPDHLPRALIGGTNKRAPGTPARFSGSLLGDKAALT |
| | | LSGVQPEDEAEYYCALWYSNLWVFGGGTKLTVLGQPKAAPSVTLFPPSSE |
| | | ELQANKATLVCLISDFYPGAVTVAWKADSSPVKAGVETTTPSKQSNNKYA |
| | | ASSYLSLTPEQWKSHRSYSCQVTHEGSTVEKTVAPTECS |
| 1LLX-F | 109 | EIVLTQSPATLSLSPGERATLSCRASQSVSSYLAWYQQKPGQAPRLLIYD |
| | | ASNRATGIPARFSGSGSGTDFTLTISSLEPEDFAVYYCQQRSNWPTFGQG |
| | | TKVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGL |
| | | SSPVTKSFNRGECGPAPLGLGGGGSQAVVTQEPSLTVSPGGTVTLTCRSS |
| | | TGAVTTSNYANWVQQKPDHLPRALIGGTNKRAPGTPARFSGSLLGDKAAL |
| | | TLSGVQPEDEAEYYCALWYSNLWVFGGGTKLTVLGQPKAAPSVTLFPPSS |
| | | EELQANKATLVCLISDFYPGAVTVAWKADSSPVKAGVETTTPSKQSNNKY |
| | | AASSYLSLTPEQWKSHRSYSCQVTHEGSTVEKTVAPTECS |
| 2LL-F | 110 | EIVLTQSPATLSLSPGERATLSCRASQSVSSYLAWYQQKPGQAPRLLIYD |
| | | ASNRATGIPARFSGSGSGTDFTLTISSLEPEDFAVYYCQQRSNWPTFGQG |
| | | TKVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGL |
| | | SSPVTKSFNRGECGPAPLGLGGGSQTVVTQEPSLTVSPGGTVTLTCRSST |
| | | GAVTTSNYPNWVQQKPGQAPRALIGGTNKRAPGTPARFSGSLLGGKAALT |
| | | LSGVQPEDEAEYYCALWYSNLWVFGGGTKLTVLGQPKAAPSVTLFPPSSE |
| | | ELQANKATLVCLISDFYPGAVTVAWKADSSPVKAGVETTTPSKQSNNKYA |
| | | ASSYLSLTPEQWKSHRSYSCQVTHEGSTVEKTVAPTECS |
| 3LL-F | 111 | EIVLTQSPATLSLSPGERATLSCRASQSVSSYLAWYQQKPGQAPRLLIYD |
| | | ASNRATGIPARFSGSGSGTDFTLTISSLEPEDFAVYYCQQRSNWPTFGQG |
| | | TKVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGL |
| | | SSPVTKSFNRGECGPAPLGLGGGSQAVVTQEPSLTVSPGGTVTLTCRSST |
| | | GAVTTSNYANWVQQKPDHLPRALIGGTNKRAPGVPARFSGSLLGDKAALT |
| | | ISGAQPEDEAIYYCALWYSNLWVFGGGTKLTVLGQPKAAPSVTLFPPSSE |
| | | ELQANKATLVCLISDFYPGAVTVAWKADSSPVKAGVETTTPSKQSNNKYA |
| | | ASSYLSLTPEQWKSHRSYSCQVTHEGSTVEKTVAPTECS |
| 4LL-F | 112 | EIVLTQSPATLSLSPGERATLSCRASQSVSSYLAWYQQKPGQAPRLLIYD |
| | | ASNRATGIPARFSGSGSGTDFTLTISSLEPEDFAVYYCQQRSNWPTFGQG |
| | | TKVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGL |
| | | SSPVTKSFNRGECGPAPLGLGGGSQAVVTQEPSLTVSPGGTVTLTCRSST |
| | | GAVTSGYYANWVQQKPDHLPRALIGGTNKRAPGTPARFSGSLLGDKAALT |
| | | LSGVQPEDEAEYYCALWYSNLWVFGGGTKLTVLGQPKAAPSVTLFPPSSE |
| | | ELQANKATLVCLISDFYPGAVTVAWKADSSPVKAGVETTTPSKQSNNKYA |
| | | ASSYLSLTPEQWKSHRSYSCQVTHEGSTVEKTVAPTECS |
| 5LL-F | 113 | EIVLTQSPATLSLSPGERATLSCRASQSVSSYLAWYQQKPGQAPRLLIYD |
| | | ASNRATGIPARFSGSGSGTDFTLTISSLEPEDFAVYYCQQRSNWPTFGQG |
| | | TKVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGL |
| | | SSPVTKSFNRGECGPAPLGLGGGSQTVVTQEPSLTVSPGGTVTLTCRSST |
| | | GAVTSGYYPNWVQQKPGQAPRALIGGTNKRAPGTPARFSGSLLGGKAALT |
| | | LSGVQPEDEAEYYCALWYSNLWVFGGGTKLTVLGQPKAAPSVTLFPPSSE |
| | | ELQANKATLVCLISDFYPGAVTVAWKADSSPVKAGVETTTPSKQSNNKYA |
| | | ASSYLSLTPEQWKSHRSYSCQVTHEGSTVEKTVAPTECS |
| 6LL-F | 114 | EIVLTQSPATLSLSPGERATLSCRASQSVSSYLAWYQQKPGQAPRLLIYD |
| | | ASNRATGIPARFSGSGSGTDFTLTISSLEPEDFAVYYCQQRSNWPTFGQG |
| | | TKVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGL |
| | | SSPVTKSFNRGECGPAPLGLGGGSQTVVTQEPSLTVSPGGTVTLTCGSST |
| | | GAVTSGYYPNWVQQKPGQAPRGLIGGTKFLAPGTPARFSGSLLGGKAALT |
| | | LSGVQPEDEAEYYCALWYSNRWVFGGGTKLTVLGQPKAAPSVTLFPPSSE |
| | | ELQANKATLVCLISDFYPGAVTVAWKADSSPVKAGVETTTPSKQSNNKYA |
| | | ASSYLSLTPEQWKSHRSYSCQVTHEGSTVEKTVAPTECS |
| 7LL-F | 115 | EIVLTQSPATLSLSPGERATLSCRASQSVSSYLAWYQQKPGQAPRLLIYD |
| | | ASNRATGIPARFSGSGSGTDFTLTISSLEPEDFAVYYCQQRSNWPTFGQG |
| | | TKVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGL |
| | | SSPVTKSFNRGECGPAPLGLGGGSQAVVTQESALTTSPGETVTLTCRSST |
| | | GAVTTSNYANWVQEKPDHLFTGLIGGTNKRAPGVPARFSGSLIGDKAALT |
| | | ITGAQTEDEAIYFCALWYSNLWVFGGGTKLTVLGQPKAAPSVTLFPPSSE |
| | | ELQANKATLVCLISDFYPGAVTVAWKADSSPVKAGVETTTPSKQSNNKYA |
| | | ASSYLSLTPEQWKSHRSYSCQVTHEGSTVEKTVAPTECS |
| 1HH | 116 | QVQLVQSGAEVKKPGSSVKVSCKTSGDTFSTYAISWVRQAPGQGLEWMGG |
| | | IIPIFGKAHYAQKFQGRVTITADESTSTAYMELSSLRSEDTAVYFCARKF |
| | | HFVSGSPFGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLG |
| | | TQTYICNVNHKPSNTKVDKKVEPKSCGPAPELLGGGSEVQLVESGGGLVQ |
| | | PGGSLRLSCAASGFTFNTYAMNWVRQAPGKGLEWVGRIRSKYNNYATYYA |
| | | DSVKDRFTISRDDSQSILYLQMNSLKTEDTAVYYCVRHGNFGNSYVSWFA |
| | | YWGQGTLVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNVNHK |
| | | PSNTKVDKKVEPKSCDKTHTCPPCPAPEAAGAPSVFLFPPKPKDTLMISR |
| | | TPEVTCVVVDVSHEDPEVKFNWYVDGVEVHNAKTKPREEQYNSTYRVVSV |
| | | LTVLHQDWLNGKEYKCKVSNKALPAPIEKTISKAKGQPREPQVYTLPPCR |
| | | DELTKNQVSLWCLVKGFYPSDIAVEWESNGQPENNYKTTPPVLDSDGSFF |
| | | LYSKLTVDKSRWQQGNVFSCSVMHEALHNHYTQKSLSLSPGK |
| 1HHX | 117 | QVQLVQSGAEVKKPGSSVKVSCKTSGDTFSTYAISWVRQAPGQGLEWMGG |
| | | IIPIFGKAHYAQKFQGRVTITADESTSTAYMELSSLRSEDTAVYFCARKF |
| | | HFVSGSPFGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLG |
| | | TQTYICNVNHKPSNTKVDKKVEPKSCGPAPELLGGGGSEVQLVESGGGLV |
| | | QPGGSLRLSCAASGFTFNTYAMNWVRQAPGKGLEWVGRIRSKYNNYATYY |
| | | ADSVKDRFTISRDDSQSILYLQMNSLKTEDTAVYYCVRHGNFGNSYVSWF |
| | | AYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNH |
| | | KPSNTKVDKKVEPKSCDKTHTCPPCPAPEAAGAPSVFLFPPKPKDTLMIS |
| | | RTPEVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYTLPPC |
| | | RDELTKNQVSLWCLVKGFYPSDIAVEWESNGQPENNYKTTPPVLDSDGSF |
| | | FLYSKLTVDKSRWQQGNVFSCSVMHEALHNHYTQKSLSLSPGK |
| 2HH | 118 | QVQLVQSGAEVKKPGSSVKVSCKTSGDTFSTYAISWVRQAPGQGLEWMGG |
| | | IIPIFGKAHYAQKFQGRVTITADESTSTAYMELSSLRSEDTAVYFCARKF |
| | | HFVSGSPFGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLG |
| | | TQTYICNVNHKPSNTKVDKKVEPKSCGPAPELLGGGSEVQLVESGGGLVQ |
| | | PGGSLRLSCAASGFTFNTYAMNWVRQAPGKGLEWVARIRSKYNNYATYYA |
| | | DSVKDRFTISRDDSQSILYLQMNSLKTEDTAVYYCVRHGNFGNSYVSWFA |
| | | YWGQGTLVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNVNHK |
| | | PSNTKVDKKVEPKSCDKTHTCPPCPAPEAAGAPSVFLFPPKPKDTLMISR |
| | | TPEVTCVVVDVSHEDPEVKFNWYVDGVEVHNAKTKPREEQYNSTYRVVSV |
| | | LTVLHQDWLNGKEYKCKVSNKALPAPIEKTISKAKGQPREPQVYTLPPCR |
| | | DELTKNQVSLWCLVKGFYPSDIAVEWESNGQPENNYKTTPPVLDSDGSFF |
| | | LYSKLTVDKSRWQQGNVFSCSVMHEALHNHYTQKSLSLSPGK |
| 2HHX | 119 | QVQLVQSGAEVKKPGSSVKVSCKTSGDTFSTYAISWVRQAPGQGLEWMGG |
| | | IIPIFGKAHYAQKFQGRVTITADESTSTAYMELSSLRSEDTAVYFCARKF |
| | | HFVSGSPFGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLG |
| | | TQTYICNVNHKPSNTKVDKKVEPKSCGPAPELLGGGGSEVQLVESGGGLV |
| | | QPGGSLRLSCAASGFTFNTYAMNWVRQAPGKGLEWVARIRSKYNNYATYY |
| | | ADSVKDRETISRDDSQSILYLQMNSLKTEDTAVYYCVRHGNFGNSYVSWF |
| | | AYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNH |
| | | KPSNTKVDKKVEPKSCDKTHTCPPCPAPEAAGAPSVFLFPPKPKDTLMIS |
| | | RTPEVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYTLPPC |
| | | RDELTKNQVSLWCLVKGFYPSDIAVEWESNGQPENNYKTTPPVLDSDGSF |
| | | FLYSKLTVDKSRWQQGNVFSCSVMHEALHNHYTQKSLSLSPGK |
| 3HH | 120 | QVQLVQSGAEVKKPGSSVKVSCKTSGDTFSTYAISWVRQAPGQGLEWMGG |
| | | IIPIFGKAHYAQKFQGRVTITADESTSTAYMELSSLRSEDTAVYFCARKF |
| | | HFVSGSPFGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLG |
| | | TQTYICNVNHKPSNTKVDKKVEPKSCGPAPELLGGGSEVQLVESGGGLVQ |
| | | PGGSLRLSCAASGFTFNTYAMNWVRQAPGKGLEWVGRIRSKYNNYATYYA |
| | | DSVKGRFTISRDDSKNSLYLQMNSLKTEDTAVYYCARHGNFGNSYVSWFA |
| | | YWGQGTLVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNVNHK |
| | | PSNTKVDKKVEPKSCDKTHTCPPCPAPEAAGAPSVFLFPPKPKDTLMISR |
| | | TPEVTCVVVDVSHEDPEVKFNWYVDGVEVHNAKTKPREEQYNSTYRVVSV |
| | | LTVLHQDWLNGKEYKCKVSNKALPAPIEKTISKAKGQPREPQVYTLPPCR |
| | | DELTKNQVSLWCLVKGFYPSDIAVEWESNGQPENNYKTTPPVLDSDGSFF |
| | | LYSKLTVDKSRWQQGNVFSCSVMHEALHNHYTQKSLSLSPGK |
| 4HH | 121 | QVQLVQSGAEVKKPGSSVKVSCKTSGDTFSTYAISWVRQAPGQGLEWMGG |
| | | IIPIFGKAHYAQKFQGRVTITADESTSTAYMELSSLRSEDTAVYFCARKF |
| | | HFVSGSPFGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLG |
| | | TQTYICNVNHKPSNTKVDKKVEPKSCGPAPELLGGGSEVQLVESGGGLVQ |
| | | PGGSLRLSCAASGFTFSTYAMNWVRQAPGKGLEWVGRIRSKYNNYATYYA |
| | | DSVKGRFTISRDDSKNSLYLQMNSLKTEDTAVYYCARHGNFGNSYVSWFA |
| | | YWGQGTLVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNVNHK |
| | | PSNTKVDKKVEPKSCDKTHTCPPCPAPEAAGAPSVFLFPPKPKDTLMISR |
| | | TPEVTCVVVDVSHEDPEVKFNWYVDGVEVHNAKTKPREEQYNSTYRVVSV |
| | | LTVLHQDWLNGKEYKCKVSNKALPAPIEKTISKAKGQPREPQVYTLPPCR |
| | | DELTKNQVSLWCLVKGFYPSDIAVEWESNGQPENNYKTTPPVLDSDGSFF |
| | | LYSKLTVDKSRWQQGNVFSCSVMHEALHNHYTQKSLSLSPGK |
| 5HH | 122 | QVQLVQSGAEVKKPGSSVKVSCKTSGDTFSTYAISWVRQAPGQGLEWMGG |
| | | IIPIFGKAHYAQKFQGRVTITADESTSTAYMELSSLRSEDTAVYFCARKF |
| | | HFVSGSPFGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLG |
| | | TQTYICNVNHKPSNTKVDKKVEPKSCGPAPELLGGGSEVQLVESGGGLVQ |
| | | PGGSLKLSCAASGFTFNKYAMNWVRQAPGKGLEWVARIRSKYNNYATYYA |
| | | DSVKDRFTISRDDSKNTAYLQMNNLKTEDTAVYYCVRHGNFGNSYISYWA |
| | | YWGQGTLVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNVNHK |
| | | PSNTKVDKKVEPKSCDKTHTCPPCPAPEAAGAPSVFLFPPKPKDTLMISR |
| | | TPEVTCVVVDVSHEDPEVKFNWYVDGVEVHNAKTKPREEQYNSTYRVVSV |
| | | LTVLHQDWLNGKEYKCKVSNKALPAPIEKTISKAKGQPREPQVYTLPPCR |
| | | DELTKNQVSLWCLVKGFYPSDIAVEWESNGQPENNYKTTPPVLDSDGSFF |
| | | LYSKLTVDKSRWQQGNVFSCSVMHEALHNHYTQKSLSLSPGK |
| 6HH | 123 | QVQLVQSGAEVKKPGSSVKVSCKTSGDTFSTYAISWVRQAPGQGLEWMGG |
| | | IIPIFGKAHYAQKFQGRVTITADESTSTAYMELSSLRSEDTAVYFCARKF |
| | | HFVSGSPFGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLG |
| | | TQTYICNVNHKPSNTKVDKKVEPKSCGPAPELLGGGSEVQLVESGGGLVQ |
| | | PKGSLKLSCAASGFTFNTYAMNWVRQAPGKGLEWVARIRSKYNNYATYYA |
| | | DSVKDRFTISRDDSQSILYLQMNNLKTEDTAMYYCVRHGNFGNSYVSWFA |
| | | YWGQGTLVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNVNHK |
| | | PSNTKVDKKVEPKSCDKTHTCPPCPAPEAAGAPSVFLFPPKPKDTLMISR |
| | | TPEVTCVVVDVSHEDPEVKFNWYVDGVEVHNAKTKPREEQYNSTYRVVSV |
| | | LTVLHQDWLNGKEYKCKVSNKALPAPIEKTISKAKGQPREPQVYTLPPCR |
| | | DELTKNQVSLWCLVKGFYPSDIAVEWESNGQPENNYKTTPPVLDSDGSFF |
| | | LYSKLTVDKSRWQQGNVFSCSVMHEALHNHYTQKSLSLSPGK |
| Fc- | 124 | SCPPCPAPEAAGAPSVFLFPPKPKDTLMISRTPEVTCVVVDVSHEDPEVK |
| stump | | FNWYVDGVEVHNAKTKPREEQYNSTYRVVSVLTVLHQDWLNGKEYKCKVS |
| | | NKALPAPIEKTISKAKGQPREPQVCTLPPSRDELTKNQVSLSCAVKGFYP |
| | | SDIAVEWESNGQPENNYKTTPPVLDSDGSFFLVSKLTVDKSRWQQGNVFS |
| | | CSVMHEALHNHYTQKSLSLSPGK |
| 1LLR- | 125 | EIVLTQSPATLSLSPGERATLSCRASQSVSSYLAWYQQKPGQAPRLLIYD |
| F | | ASNRATGIPARFSGSGSGTDFTLTISSLEPEDFAVYYCQQRSNWPTFGQG |
| | | TKVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGL |
| | | SSPVTKSFNRGECGPAPLGLRGGSQAVVTQEPSLTVSPGGTVTLTCRSST |
| | | GAVTTSNYANWVQQKPDHLPRALIGGTNKRAPGTPARFSGSLLGDKAALT |
| | | LSGVQPEDEAEYYCALWYSNLWVFGGGTKLTVLGQPKAAPSVTLFPPSSE |
| | | ELQANKATLVCLISDFYPGAVTVAWKADSSPVKAGVETTTPSKQSNNKYA |
| | | ASSYLSLTPEQWKSHRSYSCQVTHEGSTVEKTVAPTECS |
| 1LLRX- | 126 | EIVLTQSPATLSLSPGERATLSCRASQSVSSYLAWYQQKPGQAPRLLIYD |
| F | | ASNRATGIPARFSGSGSGTDFTLTISSLEPEDFAVYYCQQRSNWPTFGQG |
| | | TKVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGL |
| | | SSPVTKSFNRGECGPAPLGLRGGGSQAVVTQEPSLTVSPGGTVTLTCRSS |
| | | TGAVTTSNYANWVQQKPDHLPRALIGGTNKRAPGTPARFSGSLLGDKAAL |
| | | TLSGVQPEDEAEYYCALWYSNLWVFGGGTKLTVLGQPKAAPSVTLFPPSS |
| | | EELQANKATLVCLISDFYPGAVTVAWKADSSPVKAGVETTTPSKQSNNKY |
| | | AASSYLSLTPEQWKSHRSYSCQVTHEGSTVEKTVAPTECS |
| 3LLX- | 53 | EIVLTQSPATLSLSPGERATLSCRASQSVSSYLAWYQQKPGQAPRLLIYD |
| F | | ASNRATGIPARFSGSGSGTDFTLTISSLEPEDFAVYYCQQRSNWPTFGQG |
| | | TKVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGL |
| | | SSPVTKSFNRGECGPAPLGLGGGGSQAVVTQEPSLTVSPGGTVTLTCRSS |
| | | TGAVTTSNYANWVQQKPDHLPRALIGGTNKRAPGVPARFSGSLLGDKAAL |
| | | TISGAQPEDEAIYYCALWYSNLWVFGGGTKLTVLGQPKAAPSVTLFPPSS |
| | | EELQANKATLVCLISDFYPGAVTVAWKADSSPVKAGVETTTPSKQSNNKY |
| | | AASSYLSLTPEQWKSHRSYSCQVTHEGSTVEKTVAPTECS |
| 3LLR- | 127 | EIVLTQSPATLSLSPGERATLSCRASQSVSSYLAWYQQKPGQAPRLLIYD |
| F | | ASNRATGIPARFSGSGSGTDFTLTISSLEPEDFAVYYCQQRSNWPTFGQG |
| | | TKVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGL |
| | | SSPVTKSFNRGECGPAPLGLRGGSQAVVTQEPSLTVSPGGTVTLTCRSST |
| | | GAVTTSNYANWVQQKPDHLPRALIGGTNKRAPGVPARFSGSLLGDKAALT |
| | | ISGAQPEDEAIYYCALWYSNLWVFGGGTKLTVLGQPKAAPSVTLFPPSSE |
| | | ELQANKATLVCLISDFYPGAVTVAWKADSSPVKAGVETTTPSKQSNNKYA |
| | | ASSYLSLTPEQWKSHRSYSCQVTHEGSTVEKTVAPTECS |
| 3LLRX- | 128 | EIVLTQSPATLSLSPGERATLSCRASQSVSSYLAWYQQKPGQAPRLLIYD |
| F | | ASNRATGIPARFSGSGSGTDFTLTISSLEPEDFAVYYCQQRSNWPTFGQG |
| | | TKVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGL |
| | | SSPVTKSFNRGECGPAPLGLRGGGSQAVVTQEPSLTVSPGGTVTLTCRSS |
| | | TGAVTTSNYANWVQQKPDHLPRALIGGTNKRAPGVPARFSGSLLGDKAAL |
| | | TISGAQPEDEAIYYCALWYSNLWVFGGGTKLTVLGQPKAAPSVTLFPPSS |
| | | EELQANKATLVCLISDFYPGAVTVAWKADSSPVKAGVETTTPSKQSNNKY |
| | | AASSYLSLTPEQWKSHRSYSCQVTHEGSTVEKTVAPTECS |
| 2HHR- | 129 | QVQLVQSGAEVKKPGSSVKVSCKTSGDTFSTYAISWVRQAPGQGLEWMGG |
| F | | IIPIFGKAHYAQKFQGRVTITADESTSTAYMELSSLRSEDTAVYFCARKF |
| | | HFVSGSPFGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLG |
| | | TQTYICNVNHKPSNTKVDKKVEPKSCGPAPLGLRGGSEVQLVESGGGLVQ |
| | | PGGSLRLSCAASGFTFNTYAMNWVRQAPGKGLEWVARIRSKYNNYATYYA |
| | | DSVKDRFTISRDDSQSILYLQMNSLKTEDTAVYYCVRHGNFGNSYVSWFA |
| | | YWGQGTLVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNVNHK |
| | | PSNTKVDKKVEPKSCDKTHTCPPCPAPEAAGAPSVFLFPPKPKDTLMISR |
| | | TPEVTCVVVDVSHEDPEVKFNWYVDGVEVHNAKTKPREEQYNSTYRVVSV |
| | | LTVLHQDWLNGKEYKCKVSNKALPAPIEKTISKAKGQPREPQVYTLPPCR |
| | | DELTKNQVSLWCLVKGFYPSDIAVEWESNGQPENNYKTTPPVLDSDGSFF |
| | | LYSKLTVDKSRWQQGNVFSCSVMHEALHNHYTQKSLSLSPGK |
| 2HHRX- | 130 | QVQLVQSGAEVKKPGSSVKVSCKTSGDTFSTYAISWVRQAPGQGLEWMGG |
| F | | IIPIFGKAHYAQKFQGRVTITADESTSTAYMELSSLRSEDTAVYFCARKF |
| | | HFVSGSPFGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLG |
| | | TQTYICNVNHKPSNTKVDKKVEPKSCGPAPLGLRGGGSEVQLVESGGGLV |
| | | QPGGSLRLSCAASGFTFNTYAMNWVRQAPGKGLEWVARIRSKYNNYATYY |
| | | ADSVKDRETISRDDSQSILYLQMNSLKTEDTAVYYCVRHGNFGNSYVSWF |
| | | AYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNH |
| | | KPSNTKVDKKVEPKSCDKTHTCPPCPAPEAAGAPSVFLFPPKPKDTLMIS |
| | | RTPEVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYTLPPC |
| | | RDELTKNQVSLWCLVKGFYPSDIAVEWESNGQPENNYKTTPPVLDSDGSF |
| | | FLYSKLTVDKSRWQQGNVFSCSVMHEALHNHYTQKSLSLSPGK |
| 3HHX | 147 | QVQLVQSGAEVKKPGSSVKVSCKTSGDTFSTYAISWVRQAPGQGLEWMGG |
| | | IIPIFGKAHYAQKFQGRVTITADESTSTAYMELSSLRSEDTAVYFCARKF |
| | | HFVSGSPFGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLG |
| | | TQTYICNVNHKPSNTKVDKKVEPKSCGPAPELLGGGGSEVQLVESGGGLV |
| | | QPGGSLRLSCAASGFTFNTYAMNWVRQAPGKGLEWVGRIRSKYNNYATYY |
| | | ADSVKGRFTISRDDSKNSLYLQMNSLKTEDTAVYYCARHGNFGNSYVSWF |
| | | AYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNH |
| | | KPSNTKVDKKVEPKSCDKTHTCPPCPAPEAAGAPSVFLFPPKPKDTLMIS |
| | | RTPEVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYTLPPC |
| | | RDELTKNQVSLWCLVKGFYPSDIAVEWESNGQPENNYKTTPPVLDSDGSF |
| | | FLYSKLTVDKSRWQQGNVFSCSVMHEALHNHYTQKSLSLSPGK |
| 3HHR- | 131 | QVQLVQSGAEVKKPGSSVKVSCKTSGDTFSTYAISWVRQAPGQGLEWMGG |
| F | | IIPIFGKAHYAQKFQGRVTITADESTSTAYMELSSLRSEDTAVYFCARKF |
| | | HFVSGSPFGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLG |
| | | TQTYICNVNHKPSNTKVDKKVEPKSCGPAPLGLRGGSEVQLVESGGGLVQ |
| | | PGGSLRLSCAASGFTFNTYAMNWVRQAPGKGLEWVGRIRSKYNNYATYYA |
| | | DSVKGRFTISRDDSKNSLYLQMNSLKTEDTAVYYCARHGNFGNSYVSWFA |
| | | YWGQGTLVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNVNHK |
| | | PSNTKVDKKVEPKSCDKTHTCPPCPAPEAAGAPSVFLFPPKPKDTLMISR |
| | | TPEVTCVVVDVSHEDPEVKFNWYVDGVEVHNAKTKPREEQYNSTYRVVSV |
| | | LTVLHQDWLNGKEYKCKVSNKALPAPIEKTISKAKGQPREPQVYTLPPCR |
| | | DELTKNQVSLWCLVKGFYPSDIAVEWESNGQPENNYKTTPPVLDSDGSFF |
| | | LYSKLTVDKSRWQQGNVFSCSVMHEALHNHYTQKSLSLSPGK |
| 3HHRX- | 132 | QVQLVQSGAEVKKPGSSVKVSCKTSGDTFSTYAISWVRQAPGQGLEWMGG |
| F | | IIPIFGKAHYAQKFQGRVTITADESTSTAYMELSSLRSEDTAVYFCARKF |
| | | HFVSGSPFGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLG |
| | | TQTYICNVNHKPSNTKVDKKVEPKSCGPAPLGLRGGGSEVQLVESGGGLV |
| | | QPGGSLRLSCAASGFTFNTYAMNWVRQAPGKGLEWVGRIRSKYNNYATYY |
| | | ADSVKGRFTISRDDSKNSLYLQMNSLKTEDTAVYYCARHGNFGNSYVSWF |
| | | AYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNH |
| | | KPSNTKVDKKVEPKSCDKTHTCPPCPAPEAAGAPSVFLFPPKPKDTLMIS |
| | | RTPEVTCVVVDVSHEDPEVKFNWYVDGVEVHNAKTKPREEQYNSTYRVVS |
| | | VLTVLHQDWLNGKEYKCKVSNKALPAPIEKTISKAKGQPREPQVYTLPPC |
| | | RDELTKNQVSLWCLVKGFYPSDIAVEWESNGQPENNYKTTPPVLDSDGSF |
| | | FLYSKLTVDKSRWQQGNVFSCSVMHEALHNHYTQKSLSLSPGK |
| B- | 133 | EIVLTQSPATLSLSPGERATLSCRASQSVSSYLAWYQQKPGQAPRLLIYD |
| 1LL-F | | ASNRATGIPARFSGSGSGTDFTLTISSLEPEDFAVYYCQQRSNWPTFGQG |
| | | TKVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGL |
| | | SSPVTKSFNRGECGPAPLGLGGGSQAVVTQEPSLTVSPGGTVTLTCRSST |
| | | GAVTTSNYANWVQQKPDHLPRALIGGTNKRAPGTPARFSGSLLGDKAALT |
| | | LSGVQPEDEAEYYCALWYSNLWVFGGGTKLTVLGQPKAAPSVTLFPPSSE |
| | | ELQANKATLVCLISDFYPGAVTVAWKADSSPVKAGVETTTPSKQSNNKYA |
| | | ASSYLSLTPEQWKSHRSYSCQVTHEGSTVEKTVAPTECGGGGSGGGGSGG |
| | | GGSVFLFPPKPKDTLMISRTPEVTCVVVDVSHEDPEVKFNWYVDGVEVHN |
| | | AKTKPREEQYNSTYRVVSVLTVLHQDWLNGKEYKCKVSNKALPAPIEKTI |
| | | SKAKGQPREPQVYTLPPSRDELTKNQVSLSCAVKGFYPSDIAVEWESNGQ |
| | | PENNYKTTPPVLDSDGSFFLVSKLTVDKSRWQQGNVFSCSVMHEALHNHY |
| | | TQKSLSLSPGK |
| B- | 134 | EIVLTQSPATLSLSPGERATLSCRASQSVSSYLAWYQQKPGQAPRLLIYD |
| 1LLX- | | ASNRATGIPARFSGSGSGTDFTLTISSLEPEDFAVYYCQQRSNWPTFGQG |
| F | | TKVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGL |
| | | SSPVTKSFNRGECGPAPLGLGGGGSQAVVTQEPSLTVSPGGTVTLTCRSS |
| | | TGAVTTSNYANWVQQKPDHLPRALIGGTNKRAPGTPARFSGSLLGDKAAL |
| | | TLSGVQPEDEAEYYCALWYSNLWVFGGGTKLTVLGQPKAAPSVTLFPPSS |
| | | EELQANKATLVCLISDFYPGAVTVAWKADSSPVKAGVETTTPSKQSNNKY |
| | | AASSYLSLTPEQWKSHRSYSCQVTHEGSTVEKTVAPTECGGGGSGGGGSG |
| | | GGGSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKT |
| | | ISKAKGQPREPQVYTLPPSRDELTKNQVSLSCAVKGFYPSDIAVEWESNG |
| | | QPENNYKTTPPVLDSDGSFFLVSKLTVDKSRWQQGNVFSCSVMHEALHNH |
| | | YTQKSLSLSPGK |
| B- | 135 | EIVLTQSPATLSLSPGERATLSCRASQSVSSYLAWYQQKPGQAPRLLIYD |
| 3LL-F | | ASNRATGIPARFSGSGSGTDETLTISSLEPEDFAVYYCQQRSNWPTFGQG |
| | | TKVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGL |
| | | SSPVTKSFNRGECGPAPLGLGGGSQAVVTQEPSLTVSPGGTVTLTCRSST |
| | | GAVTTSNYANWVQQKPDHLPRALIGGTNKRAPGVPARFSGSLLGDKAALT |
| | | ISGAQPEDEAIYYCALWYSNLWVFGGGTKLTVLGQPKAAPSVTLFPPSSE |
| | | ELQANKATLVCLISDFYPGAVTVAWKADSSPVKAGVETTTPSKQSNNKYA |
| | | ASSYLSLTPEQWKSHRSYSCQVTHEGSTVEKTVAPTECGGGGSGGGGSGG |
| | | GGSVFLFPPKPKDTLMISRTPEVTCVVVDVSHEDPEVKFNWYVDGVEVHN |
| | | AKTKPREEQYNSTYRVVSVLTVLHQDWLNGKEYKCKVSNKALPAPIEKTI |
| | | SKAKGQPREPQVYTLPPSRDELTKNQVSLSCAVKGFYPSDIAVEWESNGQ |
| | | PENNYKTTPPVLDSDGSFFLVSKLTVDKSRWQQGNVFSCSVMHEALHNHY |
| | | TQKSLSLSPGK |
| B- | 136 | EIVLTQSPATLSLSPGERATLSCRASQSVSSYLAWYQQKPGQAPRLLIYD |
| 3LLX- | | ASNRATGIPARFSGSGSGTDFTLTISSLEPEDFAVYYCQQRSNWPTFGQG |
| F | | TKVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGL |
| | | SSPVTKSFNRGECGPAPLGLGGGGSQAVVTQEPSLTVSPGGTVTLTCRSS |
| | | TGAVTTSNYANWVQQKPDHLPRALIGGTNKRAPGVPARFSGSLLGDKAAL |
| | | TISGAQPEDEAIYYCALWYSNLWVFGGGTKLTVLGQPKAAPSVTLFPPSS |
| | | EELQANKATLVCLISDFYPGAVTVAWKADSSPVKAGVETTTPSKQSNNKY |
| | | AASSYLSLTPEQWKSHRSYSCQVTHEGSTVEKTVAPTECGGGGSGGGGSG |
| | | GGGSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKT |
| | | ISKAKGQPREPQVYTLPPSRDELTKNQVSLSCAVKGFYPSDIAVEWESNG |
| | | QPENNYKTTPPVLDSDGSFFLVSKLTVDKSRWQQGNVFSCSVMHEALHNH |
| | | YTQKSLSLSPGK |
| B- | 137 | QVQLVQSGAEVKKPGSSVKVSCKTSGDTFSTYAISWVRQAPGQGLEWMGG |
| 2HH | | IIPIFGKAHYAQKFQGRVTITADESTSTAYMELSSLRSEDTAVYFCARKF |
| | | HFVSGSPFGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLG |
| | | TQTYICNVNHKPSNTKVDKKVEPKSCGPAPELLGGGSEVQLVESGGGLVQ |
| | | PGGSLRLSCAASGFTFNTYAMNWVRQAPGKGLEWVARIRSKYNNYATYYA |
| | | DSVKDRETISRDDSQSILYLQMNSLKTEDTAVYYCVRHGNFGNSYVSWFA |
| | | YWGQGTLVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNVNHK |
| | | PSNTKVDKKVEPKSCGGGGSGGGGSGGGGSVFLFPPKPKDTLMISRTPEV |
| | | TCVVVDVSHEDPEVKFNWYVDGVEVHNAKTKPREEQYNSTYRVVSVLTVL |
| | | HQDWLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFFLYSK |
| | | LTVDKSRWQQGNVFSCSVMHEALHNHYTQKSLSLSPGK |
| B- | 138 | QVQLVQSGAEVKKPGSSVKVSCKTSGDTFSTYAISWVRQAPGQGLEWMGG |
| 2HHX | | IIPIFGKAHYAQKFQGRVTITADESTSTAYMELSSLRSEDTAVYFCARKF |
| | | HFVSGSPFGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLG |
| | | TQTYICNVNHKPSNTKVDKKVEPKSCGPAPELLGGGGSEVQLVESGGGLV |
| | | QPGGSLRLSCAASGFTFNTYAMNWVRQAPGKGLEWVARIRSKYNNYATYY |
| | | ADSVKDRFTISRDDSQSILYLQMNSLKTEDTAVYYCVRHGNFGNSYVSWF |
| | | AYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNH |
| | | KPSNTKVDKKVEPKSCGGGGSGGGGSGGGGSVFLFPPKPKDTLMISRTPE |
| | | VTCVVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSRDEL |
| | | TKNQVSLWCLVKGFYPSDIAVEWESNGQPENNYKTTPPVLDSDGSFFLYS |
| | | KLTVDKSRWQQGNVFSCSVMHEALHNHYTQKSLSLSPGK |
| B- | 139 | QVQLVQSGAEVKKPGSSVKVSCKTSGDTFSTYAISWVRQAPGQGLEWMGG |
| 3HH | | IIPIFGKAHYAQKFQGRVTITADESTSTAYMELSSLRSEDTAVYFCARKF |
| | | HFVSGSPFGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLG |
| | | TQTYICNVNHKPSNTKVDKKVEPKSCGPAPELLGGGSEVQLVESGGGLVQ |
| | | PGGSLRLSCAASGFTFNTYAMNWVRQAPGKGLEWVGRIRSKYNNYATYYA |
| | | DSVKGRFTISRDDSKNSLYLQMNSLKTEDTAVYYCARHGNFGNSYVSWFA |
| | | YWGQGTLVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNVNHK |
| | | PSNTKVDKKVEPKSCGGGGSGGGGSGGGGSVFLFPPKPKDTLMISRTPEV |
| | | TCVVVDVSHEDPEVKFNWYVDGVEVHNAKTKPREEQYNSTYRVVSVLTVL |
| | | HQDWLNGKEYKCKVSNKALPAPIEKTISKAKGQPREPQVYTLPPSRDELT |
| | | KNQVSLWCLVKGFYPSDIAVEWESNGQPENNYKTTPPVLDSDGSFFLYSK |
| | | LTVDKSRWQQGNVFSCSVMHEALHNHYTQKSLSLSPGK |
| B- | 140 | QVQLVQSGAEVKKPGSSVKVSCKTSGDTFSTYAISWVRQAPGQGLEWMGG |
| 3HHX | | IIPIFGKAHYAQKFQGRVTITADESTSTAYMELSSLRSEDTAVYFCARKF |
| | | HFVSGSPFGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLG |
| | | TQTYICNVNHKPSNTKVDKKVEPKSCGPAPELLGGGGSEVQLVESGGGLV |
| | | QPGGSLRLSCAASGFTFNTYAMNWVRQAPGKGLEWVGRIRSKYNNYATYY |
| | | ADSVKGRFTISRDDSKNSLYLQMNSLKTEDTAVYYCARHGNFGNSYVSWF |
| | | AYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNH |
| | | KPSNTKVDKKVEPKSCGGGGSGGGGSGGGGSVFLFPPKPKDTLMISRTPE |
| | | VTCVVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSRDEL |
| | | TKNQVSLWCLVKGFYPSDIAVEWESNGQPENNYKTTPPVLDSDGSFFLYS |
| | | KLTVDKSRWQQGNVFSCSVMHEALHNHYTQKSLSLSPGK |
| 2HHX- | 141 | QVQLVQSGAEVKKPGSSVKVSCKTSGDTFSTYAISWVRQAPGQGLEWMGG |
| HLE | | IIPIFGKAHYAQKFQGRVTITADESTSTAYMELSSLRSEDTAVYFCARKF |
| | | HFVSGSPFGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLG |
| | | TQTYICNVNHKPSNTKVDKKVEPKSCGPAPELLGGGGSEVQLVESGGGLV |
| | | QPGGSLRLSCAASGFTFNTYAMNWVRQAPGKGLEWVARIRSKYNNYATYY |
| | | ADSVKDRFTISRDDSQSILYLQMNSLKTEDTAVYYCVRHGNFGNSYVSWF |
| | | AYWGQGTLVTVSSASTKGPSVFPLAPSSKSTSGGTAALGCLVKDYFPEPV |
| | | TVSWNSGALTSGVHTCPAVLQSSGLYSLSSVVTVPSSSLGTQTYICNVNH |
| | | KPSNTKVDKKVEPKSSGGGGSC |

10069 Table 8 depicts illustrative full-length LB protein sequences as described in FIG. TD. Each chain contains, in N-terminus to C-terminus order, WT anti-PD-L1 variable domains followed by LHL and CD3 Fab variant, Fc linker and Fc fragment. In contrast to sequences described in Table 7, each chain contains heavy and light variable domains as outlined and illustrated in FIG. TD.

TABLE 8
Illustrative full-length sequences
with either heavy chains-light chain or
light chain-heavy chain combinations

| | SEQ ID | |
|---|---|---|
| Name | NO | Amino Acid sequence |
| 1HLX- | 142 | QVQLVQSGAEVKKPGSSVKVSCKTSGDTFSTYAISWVRQAPGQGLEWMGG |
| F | | IIPIFGKAHYAQKFQGRVTITADESTSTAYMELSSLRSEDTAVYFCARKF |
| | | HFVSGSPFGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLG |
| | | TQTYICNVNHKPSNTKVDKKVEPKSCGPAPLGLGGGGSQAVVTQEPSLTV |
| | | SPGGTVTLTCRSSTGAVTTSNYANWVQQKPDHLPRALIGGTNKRAPGTPA |
| | | RFSGSLLGDKAALTLSGVQPEDEAEYYCALWYSNLWVFGGGTKLTVLGQP |
| | | KAAPSVTLFPPSSEELQANKATLVCLISDFYPGAVTVAWKADSSPVKAGV |
| | | ETTTPSKQSNNKYAASSYLSLTPEQWKSHRSYSCQVTHEGSTVEKTVAPT |
| | | ECS |
| 2LHX | 143 | EIVLTQSPATLSLSPGERATLSCRASQSVSSYLAWYQQKPGQAPRLLIYD |
| | | ASNRATGIPARFSGSGSGTDFTLTISSLEPEDFAVYYCQQRSNWPTFGQG |
| | | TKVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGL |
| | | SSPVTKSFNRGECGPAPELLGGGGSEVQLVESGGGLVQPGGSLRLSCAAS |
| | | GFTFNTYAMNWVRQAPGKGLEWVARIRSKYNNYATYYADSVKDRFTISRD |
| | | DSQSILYLQMNSLKTEDTAVYYCVRHGNFGNSYVSWFAYWGQGTLVTVSS |
| | | ASTKGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKKVEP |
| | | KSCDKTHTCPPCPAPEAAGAPSVFLFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIEKTISKAKGQPREPQVYTLPPCRDELTKNQVSLWC |
| | | LVKGFYPSDIAVEWESNGQPENNYKTTPPVLDSDGSFFLYSKLTVDKSRW |
| | | QQGNVFSCSVMHEALHNHYTQKSLSLSPGK |
| 3HL-F | 144 | QVQLVQSGAEVKKPGSSVKVSCKTSGDTFSTYAISWVRQAPGQGLEWMGG |
| | | IIPIFGKAHYAQKFQGRVTITADESTSTAYMELSSLRSEDTAVYFCARKF |
| | | HFVSGSPFGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLG |
| | | TQTYICNVNHKPSNTKVDKKVEPKSCGPAPLGLGGGSQAVVTQEPSLTVS |
| | | PGGTVTLTCRSSTGAVTTSNYANWVQQKPDHLPRALIGGTNKRAPGVPAR |
| | | FSGSLLGDKAALTISGAQPEDEAIYYCALWYSNLWVFGGGTKLTVLGQPK |
| | | AAPSVTLFPPSSEELQANKATLVCLISDFYPGAVTVAWKADSSPVKAGVE |
| | | TTTPSKQSNNKYAASSYLSLTPEQWKSHRSYSCQVTHEGSTVEKTVAPTE |
| | | CS |
| 3LH | 145 | EIVLTQSPATLSLSPGERATLSCRASQSVSSYLAWYQQKPGQAPRLLIYD |
| | | ASNRATGIPARFSGSGSGTDFTLTISSLEPEDFAVYYCQQRSNWPTFGQG |
| | | TKVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGL |
| | | SSPVTKSFNRGECGPAPELLGGGSEVQLVESGGGLVQPGGSLRLSCAASG |
| | | FTFNTYAMNWVRQAPGKGLEWVGRIRSKYNNYATYYADSVKGRFTISRDD |
| | | SKNSLYLQMNSLKTEDTAVYYCARHGNFGNSYVSWFAYWGQGTLVTVSSA |
| | | STKGPSVFPLAPSSKSTSGGTAALGCLVKDYFPEPVTVSWNSGALTSGVH |
| | | TFPAVLQSSGLYSLSSVVTVPSSSLGTQTYICNVNHKPSNTKVDKKVEPK |
| | | SCDKTHTCPPCPAPEAAGAPSVFLFPPKPKDTLMISRTPEVTCVVVDVSH |
| | | EDPEVKFNWYVDGVEVHNAKTKPREEQYNSTYRVVSVLTVLHQDWLNGKE |
| | | YKCKVSNKALPAPIEKTISKAKGQPREPQVYTLPPCRDELTKNQVSLWCL |
| | | VKGFYPSDIAVEWESNGQPENNYKTTPPVLDSDGSFFLYSKLTVDKSRWQ |
| | | QGNVFSCSVMHEALHNHYTQKSLSLSPGK |
| 1HLX- | 146 | QVQLVQSGAEVKKPGSSVKVSCKTSGDTFSTYAISWVRQAPGQGLEWMGG |
| R | | IIPIFGKAHYAQKFQGRVTITADESTSTAYMELSSLRSEDTAVYFCARKF |
| | | HFVSGSPFGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLG |
| | | TQTYICNVNHKPSNTKVDKKVEPKSCGPAPLGLRGGGSQAVVTQEPSLTV |
| | | SPGGTVTLTCRSSTGAVTTSNYANWVQQKPDHLPRALIGGTNKRAPGTPA |
| | | RFSGSLLGDKAALTLSGVQPEDEAEYYCALWYSNLWVFGGGTKLTVLGQP |
| | | KAAPSVTLFPPSSEELQANKATLVCLISDFYPGAVTVAWKADSSPVKAGV |
| | | ETTTPSKQSNNKYAASSYLSLTPEQWKSHRSYSCQVTHEGSTVEKTVAPT |
| | | ECS |
| 3HL- | 148 | QVQLVQSGAEVKKPGSSVKVSCKTSGDTFSTYAISWVRQAPGQGLEWMGG |
| FR | | IIPIFGKAHYAQKFQGRVTITADESTSTAYMELSSLRSEDTAVYFCARKF |
| | | HFVSGSPFGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLG |
| | | TQTYICNVNHKPSNTKVDKKVEPKSCGPAPLGLRGGSQAVVTQEPSLTVS |
| | | PGGTVTLTCRSSTGAVTTSNYANWVQQKPDHLPRALIGGTNKRAPGVPAR |
| | | FSGSLLGDKAALTISGAQPEDEAIYYCALWYSNLWVFGGGTKLTVLGQPK |
| | | AAPSVTLFPPSSEELQANKATLVCLISDFYPGAVTVAWKADSSPVKAGVE |
| | | TTTPSKQSNNKYAASSYLSLTPEQWKSHRSYSCQVTHEGSTVEKTVAPTE |
| | | CS |
| 1HLX | 150 | QVQLVQSGAEVKKPGSSVKVSCKTSGDTFSTYAISWVRQAPGQGLEWMGG |
| | | IIPIFGKAHYAQKFQGRVTITADESTSTAYMELSSLRSEDTAVYFCARKF |
| | | HFVSGSPFGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLG |
| | | TQTYICNVNHKPSNTKVDKKVEPKSCGPAPELLGGGGSQAVVTQEPSLTV |
| | | SPGGTVTLTCRSSTGAVTTSNYANWVQQKPDHLPRALIGGTNKRAPGTPA |
| | | RFSGSLLGDKAALTLSGVQPEDEAEYYCALWYSNLWVFGGGTKLTVLGQP |
| | | KAAPSVTLFPPSSEELQANKATLVCLISDFYPGAVTVAWKADSSPVKAGV |
| | | ETTTPSKQSNNKYAASSYLSLTPEQWKSHRSYSCQVTHEGSTVEKTVAPT |
| | | ECS |
| 2LHX- | 151 | EIVLTQSPATLSLSPGERATLSCRASQSVSSYLAWYQQKPGQAPRLLIYD |
| R | | ASNRATGIPARFSGSGSGTDFTLTISSLEPEDFAVYYCQQRSNWPTFGQG |
| | | TKVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGL |
| | | SSPVTKSFNRGECGPAPLGLRGGGSEVQLVESGGGLVQPGGSLRLSCAAS |
| | | GFTFNTYAMNWVRQAPGKGLEWVARIRSKYNNYATYYADSVKDRFTISRD |
| | | DSQSILYLQMNSLKTEDTAVYYCVRHGNFGNSYVSWFAYWGQGTLVTVSS |
| | | ASTKGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKKVEP |
| | | KSCDKTHTCPPCPAPEAAGAPSVFLFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIEKTISKAKGQPREPQVYTLPPCRDELTKNQVSLWC |
| | | LVKGFYPSDIAVEWESNGQPENNYKTTPPVLDSDGSFFLYSKLTVDKSRW |
| | | QQGNVFSCSVMHEALHNHYTQKSLSLSPGK |
| 3HL | 152 | QVQLVQSGAEVKKPGSSVKVSCKTSGDTFSTYAISWVRQAPGQGLEWMGG |
| | | IIPIFGKAHYAQKFQGRVTITADESTSTAYMELSSLRSEDTAVYFCARKF |
| | | HFVSGSPFGMDVWGQGTTVTVSSASTKGPSVFPLAPSSKSTSGGTAALGC |
| | | LVKDYFPEPVTVSWNSGALTSGVHTFPAVLQSSGLYSLSSVVTVPSSSLG |
| | | TQTYICNVNHKPSNTKVDKKVEPKSCGPAPELLGGGSQAVVTQEPSLTVS |
| | | PGGTVTLTCRSSTGAVTTSNYANWVQQKPDHLPRALIGGTNKRAPGVPAR |
| | | FSGSLLGDKAALTISGAQPEDEAIYYCALWYSNLWVFGGGTKLTVLGQPK |
| | | AAPSVTLFPPSSEELQANKATLVCLISDFYPGAVTVAWKADSSPVKAGVE |
| | | TTTPSKQSNNKYAASSYLSLTPEQWKSHRSYSCQVTHEGSTVEKTVAPTE |
| | | CS |
| 3LHRX- | 153 | EIVLTQSPATLSLSPGERATLSCRASQSVSSYLAWYQQKPGQAPRLLIYD |
| F | | ASNRATGIPARFSGSGSGTDFTLTISSLEPEDFAVYYCQQRSNWPTFGQG |
| | | TKVEIKRTVAAPSVFIFPPSDEQLKSGTASVVCLLNNFYPREAKVQWKVD |
| | | NALQSGNSQESVTEQDSKDSTYSLSSTLTLSKADYEKHKVYACEVTHQGL |
| | | SSPVTKSFNRGECGPAPLGLRGGGSEVQLVESGGGLVQPGGSLRLSCAAS |
| | | GFTFNTYAMNWVRQAPGKGLEWVGRIRSKYNNYATYYADSVKGRFTISRD |
| | | DSKNSLYLQMNSLKTEDTAVYYCARHGNFGNSYVSWFAYWGQGTLVTVSS |
| | | ASTKGPSVFPLAPSSKSTSGGTAALGCLVKDYFPEPVTVSWNSGALTSGV |
| | | HTFPAVLQSSGLYSLSSVVTVPSSSLGTQTYICNVNHKPSNTKVDKKVEP |
| | | KSCDKTHTCPPCPAPEAAGAPSVFLFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIEKTISKAKGQPREPQVYTLPPCRDELTKNQVSLWC |
| | | LVKGFYPSDIAVEWESNGQPENNYKTTPPVLDSDGSFFLYSKLTVDKSRW |
| | | QQGNVFSCSVMHEALHNHYTQKSLSLSPGK |

The anti-PD-L1/anti-CD3 protein design may be based on sequences derived from IgG1, IgG2, IgG3, IgG4, IgE, IgM, or IgA and may or may not have effector function capacity.

In some embodiment, a protein disclosed herein comprises an Fc fragment. In some embodiments, a protein disclosed herein does not comprise an Fc fragment.

In some embodiments, a protein disclosed herein is fused or conjugated to a moiety that provides half-life extension (“HLE”). HLE may be achieved via PEGylation or through an alternative mechanism, such as (but not limited to) addition of an albumin binding domain. In some embodiments, a protein disclosed herein does not comprise an Fc fragment but is fused or conjugated to a moiety that provides HLE. In some embodiments, the moiety that provides HLE is fused to the heavy chain. In some embodiments, the moiety that provides HLE is fused to the light chain.

Proteins disclosed herein comprise domains and regions of antibody molecules. The term “antibody” broadly refers to an immunoglobulin (Ig) molecule, generally, comprising four polypeptide chains, two heavy (H) chains and two light (L) chains, or any functional fragment, mutant, variant, or derivative thereof, that retains the essential target binding features of an Ig molecule. Such mutant, variant, or derivative antibody formats are known in the art.

In a full-length antibody, each heavy chain comprises a heavy chain variable domain (abbreviated herein as VH domain) and a heavy chain constant region. The heavy chain constant region comprises three domains, CH1, CH2 and CH3. IgG, IgA, and IgD constant regions comprise a flexible hinge region between the CH1 domain and the CH2 domain. Each light chain comprises a light chain variable domain (abbreviated herein as VL domain) and a light chain constant region. The light chain constant region comprises one domain, CL. The VH and VL domains can be further subdivided into regions of hypervariability, termed complementarity determining regions (CDRs), interspersed with regions that are more conserved, termed framework regions (FRs). Each VH domain and VL domain is composed of three CDRs and four FRs, arranged from amino-terminus to carboxyl-terminus in the following order: FR1, CDR1, FR2, CDR2, FR3, CDR3, FR4.

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is according to the EU index as in Kdbat. The Fc region of an immunoglobulin generally comprises two constant domains, CH2 and CH3. An Fc region can be present in dimer or monomeric form. The Fc region binds to various cell receptors, such as Fc receptors, and other immune molecules, such as complement proteins.

A protein provided herein comprises two Fab fragments. A Fab fragment is a monovalent antigen-binding fragment consisting of the VL, VH, CL and CH1 domains.

Immunoglobulin molecules can be of any type (e.g., IgG, IgE, IgM, IgD, IgA or IgY) and class (e.g., IgG1, IgG2, IgG3, IgG4, IgA1 or IgA2) or subclass. IgG, IgD, and IgE antibodies generally contain two identical heavy chains and two identical light chains and two antigen combining domains, each composed of a VH) and a VL. Generally, IgA antibodies are composed of two monomers, each monomer composed of two heavy chains and two light chains (as for IgG, IgD, and IgE antibodies); in this way the IgA molecule has four antigen binding domains, each again composed of a VH and a VL. Certain IgA antibodies are monomeric in that they are composed of two heavy chains and two light chains. Secreted IgM antibodies are generally composed of five monomers, each monomer composed of two heavy chains and two light chains (as for IgG and IgE antibodies). Thus, the IgM molecule has ten antigen binding domains, each again composed of a VH and a VL. A cell surface form of IgM has a two heavy chain/two light chain structure similar to IgG, IgD and IgE antibodies.

As used herein, the terms “immunological binding” and “immunological binding properties” refer to the non-covalent interactions of the type which occur between an immunoglobulin molecule (e.g., antibody or antigen-binding portion thereof), or a protein comprising an immunoglobulin-derived binding domain(s) and an antigen for which the immunoglobulin or protein is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See, Malmqvist, Nature 361:186-187 (1993)). The ratio of K_{off}/Kₒₙ enables the cancellation of all parameters not related to affinity and is equal to the dissociation constant K_{d}. (See, Davies et al. (1990) Annual Rev Biochem 59:439-473). An antibody or antigen-binding portion provided herein is said to specifically bind PD-L1 or CD3 when the equilibrium binding constant (K_{d}) is ≤10 μM, preferably ≤10 nM, more preferably ≤10 nM, and most preferably ≤100 μM to about 1 μM, as measured by assays such as radioligand binding assays or similar assays known to those skilled in the art. One method for determining the K_{d} of an antibody is by using surface plasmon resonance (SPR), typically using a biosensor system such as a Biacore® system.

Functionally, the binding affinity of a protein provided herein may be within the range of 10⁻⁵M to 10⁻¹² M. For example, the binding affinity of a protein provided herein is from 10⁻⁶ M to 10⁻¹² M, from 10⁻⁷ M to 10⁻¹² M, from 10⁻⁸ M to 10⁻¹² M, from 10⁻⁹ M to 10⁻¹² M, from 10⁻⁵ M to 10⁻¹ M, from 10⁻⁶ M to 10⁻¹¹ M, from 10⁻⁷ M to 10⁻¹¹ M, from 10⁻⁸ M to 10⁻¹¹ M, from 10⁻⁹M to 10⁻¹¹ M, from 10⁻¹⁰ M to 10⁻¹¹ M, from 10⁻⁵ M to 10⁻¹⁰ M, from 10⁻⁶ M to 10⁻¹⁰ M, from 10⁻⁷ M to 10⁻¹⁰ M, from 10⁻⁸ M to 10⁻¹⁰ M, from 10⁻⁹ M to 10⁻¹ M, from 10⁻⁵ M to 10⁻⁹ M, from 10⁻⁶ M to 10⁻⁹ M, from 10⁻⁷ M to 10⁻⁹ M, from 10⁻⁸ M to 10⁻⁹ M, from 10⁻⁵ M to 10⁻⁸ M, from 10⁻⁶ M to 10⁻⁸ M, from 10⁻⁷ M to 10⁻⁸ M, from 10⁻⁵ M to 10⁻⁷ M, from 10⁻⁶ M to 10⁻⁷ M or from 10⁻⁵ M to 10⁻⁶ M.

Provided herein is a protein comprising a first polypeptide chain comprising a heavy chain and a second polypeptide chain comprising a light chain, wherein the heavy chain comprises, in N-terminus to C-terminus order, an anti-PD-L1 heavy chain variable (VH) domain, a first CH1 domain, a first linker, an anti-CD3 VH domain, and a second CH1 domain; wherein the light chain comprises, in N-terminus to C-terminus order, an anti-PD-L1 light chain variable (VL) domain, a first immunoglobulin light chain constant region, a second linker, an anti-CD3 VL domain, and a second immunoglobulin light chain constant region.

Provided herein is a protein comprising a first polypeptide chain comprising a heavy chain and a second polypeptide chain comprising a light chain, wherein the heavy chain comprises, in N-terminus to C-terminus order, an anti-PD-L1 heavy chain variable (VH) domain, a first CH1 domain, a first linker, an anti-CD3 light chain variable (VL) domain, and a first immunoglobulin light chain constant region, and wherein the light chain comprises, in N-terminus to C-terminus order, anti-PD-L1 VL domain, a second immunoglobulin light chain constant region, a second linker, an anti-CD3 VH domain, and a second CH1 domain.

In some embodiments, the heavy chain comprises in N-terminus to C-terminus order, the anti-PD-L1 VH domain, the first CH1 domain, the first linker, the anti-CD3 VH domain, the second CH1 domain, a hinge, a CH2 domain, and a CH3 domain.

In some embodiments, the protein further comprises a third polypeptide chain comprising a hinge and a Fc region. In some embodiments, the third polypeptide chain comprises the amino acid sequence of SEQ ID NO: 124.

Provided herein is a protein comprising a first polypeptide chain comprising a heavy chain and a second polypeptide chain comprising a light chain, wherein the heavy chain comprises, in N-terminus to C-terminus order, an anti-PD-L1 heavy chain variable (VH) domain, a first CH1 domain, a first linker, an anti-CD3 VH domain, a second CH1 domain, a first hinge, a first CH2 domain, and a first CH3 domain; and wherein the light chain comprises, in N-terminus to C-terminus order, an anti-PD-L1 light chain variable (VL) domain, a first immunoglobulin light chain constant region, a second linker, an anti-CD3 VL domain, a second immunoglobulin light chain constant region, a second hinge, a second CH2 domain, and a second CH3 domain.

In some embodiments, the first linker comprises the amino acid sequence of any one of SEQ ID NOs: 1-12. In some embodiments, the first linker comprises an amino acid sequence that is at least 95%, 96%, 97%, 98% or 99% identical to the amino acid sequence of any one of SEQ ID NOs: 1-12.

In some embodiments, the second linker comprises the amino acid sequence of any one of SEQ ID NOs: 1-12. In some embodiments, the second linker comprises an amino acid sequence that is at least 95%, 96%, 97%, 98% or 99% identical to the amino acid sequence of any one of SEQ ID NOs: 1-12.

In some embodiments, the first linker comprises the amino acid sequence of any one of SEQ ID NOs: 1-12, and the second linker comprises the amino acid sequence of any one of SEQ ID NOs: 1-12. In some embodiments, the first linker comprises an amino acid sequence that is at least 95%, 96%, 97%, 98% or 99% identical to the amino acid sequence of any one of SEQ ID NOs: 1-12, and the second linker comprises an amino acid sequence that is at least 95%, 96%, 97%, 98% or 99% identical to the amino acid sequence of any one of SEQ ID NOs: 1-12.

In some embodiments, the first linker is the same as the second linker. In some embodiments, the first linker is not the same as the second linker.

In some embodiments, the anti-PD-L1 VH domain comprises a HCDR1 comprising the amino acid sequence of SEQ ID NO: 50. In some embodiments, the anti-PD-L1 VH domain comprises a HCDR2 comprising the amino acid sequence of SEQ ID NO: 51. In some embodiments, the anti-PD-L1 VH domain comprises a HCDR3 comprising the amino acid sequence of SEQ ID NO: 52.

In some embodiments, the anti-PD-L1 VH domain comprises a HCDR1 comprising the amino acid sequence of SEQ ID NO: 20, a HCDR2 comprising the amino acid sequence of SEQ ID NO: 21, and a HCDR3 comprising the amino acid sequence of SEQ ID NO: 22; and the anti-PD-L1 VL domain comprises a LCDR1 comprising the amino acid sequence of SEQ ID NO: 23, a LCDR2 comprising the amino acid sequence of SEQ ID NO: 24, and a LCDR3 comprising the amino acid sequence of SEQ ID NO: 25.

In some embodiments, the anti-PD-L1 VH domain comprises a HCDR1 comprising the amino acid sequence of SEQ ID NO: 20, a HCDR2 comprising the amino acid sequence of SEQ ID NO: 21, and a HCDR3 comprising the amino acid sequence of SEQ ID NO: 22; and the anti-PD-L1 VL domain comprises at least one LCDR sequence selected from: a LCDR1 comprising the amino acid sequence of SEQ ID NO: 23, a LCDR2 comprising the amino acid sequence of SEQ ID NO: 24, and a LCDR3 comprising the amino acid sequence of SEQ ID NO: 25.

In some embodiments, the anti-PD-L1 VH domain comprises at least one HCDR sequence selected from: a HCDR1 comprising the amino acid sequence of SEQ ID NO: 20, a HCDR2 comprising the amino acid sequence of SEQ ID NO: 21, and a HCDR3 comprising the amino acid sequence of SEQ ID NO: 22; and the anti-PD-L1 VL domain comprises a LCDR1 comprising the amino acid sequence of SEQ ID NO: 23, a LCDR2 comprising the amino acid sequence of SEQ ID NO: 24, and a LCDR3 comprising the amino acid sequence of SEQ ID NO: 25.

In some embodiments, the anti-CD3 VH domain comprises a HCDR1 comprising the amino acid sequence of SEQ ID NO: 26, a HCDR2 comprising the amino acid sequence of SEQ ID NO: 27, and a HCDR3 comprising the amino acid sequence of SEQ ID NO: 29; and the anti-CD3 VL domain comprises a LCDR1 comprising the amino acid sequence of SEQ ID NO: 30, a LCDR2 comprising the amino acid sequence of SEQ ID NO: 31, and a LCDR3 comprising the amino acid sequence of SEQ ID NO: 32.

In some embodiments, the anti-CD3 VH domain comprises a HCDR1 comprising the amino acid sequence of SEQ ID NO: 26, a HCDR2 comprising the amino acid sequence of SEQ ID NO: 27, and a HCDR3 comprising the amino acid sequence of SEQ ID NO: 29; and the anti-CD3 VL domain comprises at least one LCDR sequence selected from: a LCDR1 comprising the amino acid sequence of SEQ ID NO: 30, a LCDR2 comprising the amino acid sequence of SEQ ID NO: 31, and a LCDR3 comprising the amino acid sequence of SEQ ID NO: 32.

In some embodiments, the anti-CD3 VH domain comprises at least one HCDR sequence selected from: a HCDR1 comprising the amino acid sequence of SEQ ID NO: 26, a HCDR2 comprising the amino acid sequence of SEQ ID NO: 27, and a HCDR3 comprising the amino acid sequence of SEQ ID NO: 29; and the anti-CD3 VL domain comprises a LCDR1 comprising the amino acid sequence of SEQ ID NO: 30, a LCDR2 comprising the amino acid sequence of SEQ ID NO: 31, and a LCDR3 comprising the amino acid sequence of SEQ ID NO: 32.

In some embodiments, the anti-CD3 VH domain comprises a HCDR1 comprising the amino acid sequence of SEQ ID NO: 26, a HCDR2 comprising the amino acid sequence of SEQ ID NO: 28, and a HCDR3 comprising the amino acid sequence of SEQ ID NO: 29; and the anti-CD3 VL domain comprises a LCDR1 comprising the amino acid sequence of SEQ ID NO: 30, a LCDR2 comprising the amino acid sequence of SEQ ID NO: 31, and a LCDR3 comprising the amino acid sequence of SEQ ID NO: 32.

In some embodiments, the anti-CD3 VH domain comprises a HCDR1 comprising the amino acid sequence of SEQ ID NO: 26, a HCDR2 comprising the amino acid sequence of SEQ ID NO: 28, and a HCDR3 comprising the amino acid sequence of SEQ ID NO: 29; and the anti-CD3 VL domain comprises at least one LCDR sequence selected from: a LCDR1 comprising the amino acid sequence of SEQ ID NO: 30, a LCDR2 comprising the amino acid sequence of SEQ ID NO: 31, and a LCDR3 comprising the amino acid sequence of SEQ ID NO: 32.

In some embodiments, the anti-CD3 VH domain comprises at least one HCDR sequence selected from: a HCDR1 comprising the amino acid sequence of SEQ ID NO: 26, a HCDR2 comprising the amino acid sequence of SEQ ID NO: 28, and a HCDR3 comprising the amino acid sequence of SEQ ID NO: 29; and the anti-CD3 VL domain comprises a LCDR1 comprising the amino acid sequence of SEQ ID NO: 30, a LCDR2 comprising the amino acid sequence of SEQ ID NO: 31, and a LCDR3 comprising the amino acid sequence of SEQ ID NO: 32.

In some embodiments, the anti-PD-L1 VH domain comprises a HCDR1 comprising the amino acid sequence of SEQ ID NO: 20, a HCDR2 comprising the amino acid sequence of SEQ ID NO: 21, and a HCDR3 comprising the amino acid sequence of SEQ ID NO: 22; the anti-PD-L1 VL domain comprises a LCDR1 comprising the amino acid sequence of SEQ ID NO: 23, a LCDR2 comprising the amino acid sequence of SEQ ID NO: 24, and a LCDR3 comprising the amino acid sequence of SEQ ID NO: 25; the anti-CD3 VH domain comprises a HCDR1 comprising the amino acid sequence of SEQ ID NO: 26, a HCDR2 comprising the amino acid sequence of SEQ ID NO: 27, and a HCDR3 comprising the amino acid sequence of SEQ ID NO: 29; and the anti-CD3 VL domain comprises a LCDR1 comprising the amino acid sequence of SEQ ID NO: 30, a LCDR2 comprising the amino acid sequence of SEQ ID NO: 31, and a LCDR3 comprising the amino acid sequence of SEQ ID NO: 32.

In some embodiments, the anti-PD-L1 VH domain comprises a HCDR1 comprising the amino acid sequence of SEQ ID NO: 20, a HCDR2 comprising the amino acid sequence of SEQ ID NO: 21, and a HCDR3 comprising the amino acid sequence of SEQ ID NO: 22; the anti-PD-L1 VL domain comprises a LCDR1 comprising the amino acid sequence of SEQ ID NO: 23, a LCDR2 comprising the amino acid sequence of SEQ ID NO: 24, and a LCDR3 comprising the amino acid sequence of SEQ ID NO: 25; the anti-CD3 VH domain comprises a HCDR1 comprising the amino acid sequence of SEQ ID NO: 26, a HCDR2 comprising the amino acid sequence of SEQ ID NO: 28, and a HCDR3 comprising the amino acid sequence of SEQ ID NO: 29; and the anti-CD3 VL domain comprises a LCDR1 comprising the amino acid sequence of SEQ ID NO: 30, a LCDR2 comprising the amino acid sequence of SEQ ID NO: 31, and a LCDR3 comprising the amino acid sequence of SEQ ID NO: 32.

Provided herein is a protein comprising a first polypeptide chain comprising a heavy chain and a second polypeptide chain comprising a light chain, wherein the heavy chain comprises, in N-terminus to C-terminus order, an anti-PD-L1 heavy chain variable (VH) domain, a first CH1 domain, a first linker, an anti-CD3 VH domain, and a second CH1 domain; wherein the light chain comprises, in N-terminus to C-terminus order, an anti-PD-L1 light chain variable (VL) domain, a first immunoglobulin light chain constant region, a second linker, an anti-CD3 VL domain, and a second immunoglobulin light chain constant region; wherein the anti-PD-L1 VH domain comprises a HCDR1 comprising the amino acid sequence of SEQ ID NO: 20, a HCDR2 comprising the amino acid sequence of SEQ ID NO: 21, and a HCDR3 comprising the amino acid sequence of SEQ ID NO: 22; the anti-PD-L1 VL domain comprises a LCDR1 comprising the amino acid sequence of SEQ ID NO: 23, a LCDR2 comprising the amino acid sequence of SEQ ID NO: 24, and a LCDR3 comprising the amino acid sequence of SEQ ID NO: 25; the anti-CD3 VH domain comprises a HCDR1 comprising the amino acid sequence of SEQ ID NO: 26, a HCDR2 comprising the amino acid sequence of SEQ ID NO: 27, and a HCDR3 comprising the amino acid sequence of SEQ ID NO: 29; and the anti-CD3 VL domain comprises a LCDR1 comprising the amino acid sequence of SEQ ID NO: 30, a LCDR2 comprising the amino acid sequence of SEQ ID NO: 31, and a LCDR3 comprising the amino acid sequence of SEQ ID NO: 32; wherein the first linker comprises the amino acid sequence of any one of SEQ ID NOs: 1-12; and wherein the second linker comprises the amino acid sequence of any one of SEQ ID NOs: 1-12.

Provided herein is a protein comprising a first polypeptide chain comprising a heavy chain and a second polypeptide chain comprising a light chain, wherein the heavy chain comprises, in N-terminus to C-terminus order, an anti-PD-L1 heavy chain variable (VH) domain, a first CH1 domain, a first linker, an anti-CD3 VH domain, and a second CH1 domain; wherein the light chain comprises, in N-terminus to C-terminus order, an anti-PD-L1 light chain variable (VL) domain, a first immunoglobulin light chain constant region, a second linker, an anti-CD3 VL domain, and a second immunoglobulin light chain constant region; wherein the anti-PD-L1 VH domain comprises a HCDR1 comprising the amino acid sequence of SEQ ID NO: 20, a HCDR2 comprising the amino acid sequence of SEQ ID NO: 21, and a HCDR3 comprising the amino acid sequence of SEQ ID NO: 22; the anti-PD-L1 VL domain comprises a LCDR1 comprising the amino acid sequence of SEQ ID NO: 23, a LCDR2 comprising the amino acid sequence of SEQ ID NO: 24, and a LCDR3 comprising the amino acid sequence of SEQ ID NO: 25; the anti-CD3 VH domain comprises a HCDR1 comprising the amino acid sequence of SEQ ID NO: 26, a HCDR2 comprising the amino acid sequence of SEQ ID NO: 28, and a HCDR3 comprising the amino acid sequence of SEQ ID NO: 29; and the anti-CD3 VL domain comprises a LCDR1 comprising the amino acid sequence of SEQ ID NO: 30, a LCDR2 comprising the amino acid sequence of SEQ ID NO: 31, and a LCDR3 comprising the amino acid sequence of SEQ ID NO: 32; wherein the first linker comprises the amino acid sequence of any one of SEQ ID NOs: 1-12; and wherein the second linker comprises the amino acid sequence of any one of SEQ ID NOs: 1-12.

In some embodiments, the anti-PD-L1 VH domain comprises the amino acid sequence of any one of SEQ ID NOs: 33 and 54-107. In some embodiments, the anti-PD-L1 VH domain comprises an amino acid sequence that is at least 90%, 91%, 92%, 93%, 94%, 95%, 96%, 97%, 98% or 99% identical to the amino acid sequence of any one of SEQ ID NOs: 33 and 54-107.

In some embodiments, the anti-PD-L1 VL domain comprises the amino acid sequence of SEQ ID NO: 34. In some embodiments, the anti-PD-L1 VL domain comprises an amino acid sequence that is at least 90%, 91%, 92%, 93%, 94%, 95%, 96%, 97%, 98% or 99% identical to the amino acid sequence of SEQ ID NO: 34.

In some embodiments, the anti-PD-L1 VH domain comprises the amino acid sequence of any one of SEQ ID NOs: 33 and 54-107, and the anti-PD-L1 VL domain comprises the amino acid sequence of SEQ ID NO: 34.

In some embodiments, the anti-CD3 VH domain comprises the amino acid sequence of any one of SEQ ID NOs: 44-48. In some embodiments, the anti-CD3 VH domain comprises an amino acid sequence that is at least 90%, 91%, 92%, 93%, 94%, 95%, 96%, 97%, 98% or 99% identical to the amino acid sequence of any one of SEQ ID NOs: 44-48.

In some embodiments, the anti-CD3 VL domain comprises the amino acid sequence of any one of SEQ ID NOs: 37-42. In some embodiments, the anti-CD3 VL domain comprises an amino acid sequence that is at least 90%, 91%, 92%, 93%, 94%, 95%, 96%, 97%, 98% or 99% identical to the amino acid sequence of any one of SEQ ID NOs: 37-42.

In some embodiments, the anti-CD3 VH domain comprises the amino acid sequence of any one of SEQ ID NOs: 44-48, and the anti-CD3 VL domain comprises the amino acid sequence of any one of SEQ ID NOs: 37-42.

In some embodiments, the heavy chain comprises the amino acid sequence of any one of SEQ ID NOs: 116-123, 129-132, 137-141, 142, 144, 146, 147, 148, 150, and 152. In some embodiments, the heavy chain comprises an amino acid sequence that is at least 90%, 91%, 92%, 93%, 94%, 95%, 96%, 97%, 98% or 99% identical to the amino acid sequence of any one of SEQ ID NOs: 116-123, 129-132, 137-141, 142, 144, 146, 147, 148, 150, and 152.

In some embodiments, the light chain comprises the amino acid sequence of any one of SEQ ID NOs: 53, 108-115, 125-128, and 133-136, 143, 145, 151, and 153. In some embodiments, the heavy chain comprises an amino acid sequence that is at least 90%, 91%, 92%, 93%, 94%, 95%, 96%, 97%, 98% or 99% identical to the amino acid sequence of any one of SEQ ID NOs: 53, 108-115, 125-128, and 133-136, 143, 145, 151, and 153.

In some embodiments, provided herein is a protein wherein:
(a) the heavy chain comprises the amino acid sequence of SEQ ID NO: 122, the light chain comprises the amino acid sequence of SEQ ID NO: 114, and the third polypeptide chain comprises the amino acid sequence of SEQ ID NO: 124;
(b) the heavy chain comprises the amino acid sequence of SEQ ID NO: 123, the light chain comprises the amino acid sequence of SEQ ID NO: 115, and the third polypeptide chain comprises the amino acid sequence of SEQ ID NO: 124;
(c) the heavy chain comprises the amino acid sequence of SEQ ID NO: 116, the light chain comprises the amino acid sequence of SEQ ID NO: 108, and the third polypeptide chain comprises the amino acid sequence of SEQ ID NO: 124;
(d) the heavy chain comprises the amino acid sequence of SEQ ID NO: 117, the light chain comprises the amino acid sequence of SEQ ID NO: 109, and the third polypeptide chain comprises the amino acid sequence of SEQ ID NO: 124;
(e) the heavy chain comprises the amino acid sequence of SEQ ID NO: 118, the light chain comprises the amino acid sequence of SEQ ID NO: 108, and the third polypeptide chain comprises the amino acid sequence of SEQ ID NO: 124;
(f) the heavy chain comprises the amino acid sequence of SEQ ID NO: 119, the light chain comprises the amino acid sequence of SEQ ID NO: 109, and the third polypeptide chain comprises the amino acid sequence of SEQ ID NO: 124;
(g) the heavy chain comprises the amino acid sequence of SEQ ID NO: 120, the light chain comprises the amino acid sequence of SEQ ID NO: 108, and the third polypeptide chain comprises the amino acid sequence of SEQ ID NO: 124;
(h) the heavy chain comprises the amino acid sequence of SEQ ID NO: 116, the light chain comprises the amino acid sequence of SEQ ID NO: 110, and the third polypeptide chain comprises the amino acid sequence of SEQ ID NO: 124;
(i) the heavy chain comprises the amino acid sequence of SEQ ID NO: 118, the light chain comprises the amino acid sequence of SEQ ID NO: 110, and the third polypeptide chain comprises the amino acid sequence of SEQ ID NO: 124;
(j) the heavy chain comprises the amino acid sequence of SEQ ID NO: 120, the light chain comprises the amino acid sequence of SEQ ID NO: 110, and the third polypeptide chain comprises the amino acid sequence of SEQ ID NO: 124;
(k) the heavy chain comprises the amino acid sequence of SEQ ID NO: 116, the light chain comprises the amino acid sequence of SEQ ID NO: 111, and the third polypeptide chain comprises the amino acid sequence of SEQ ID NO: 124;
(l) the heavy chain comprises the amino acid sequence of SEQ ID NO: 118, the light chain comprises the amino acid sequence of SEQ ID NO: 111, and the third polypeptide chain comprises the amino acid sequence of SEQ ID NO: 124;
(m) the heavy chain comprises the amino acid sequence of SEQ ID NO: 120, the light chain comprises the amino acid sequence of SEQ ID NO: 111, and the third polypeptide chain comprises the amino acid sequence of SEQ ID NO: 124;
(n) the heavy chain comprises the amino acid sequence of SEQ ID NO: 116, the light chain comprises the amino acid sequence of SEQ ID NO: 112, and the third polypeptide chain comprises the amino acid sequence of SEQ ID NO: 124;
(o) the heavy chain comprises the amino acid sequence of SEQ ID NO: 116, the light chain comprises the amino acid sequence of SEQ ID NO: 113, and the third polypeptide chain comprises the amino acid sequence of SEQ ID NO: 124;
(p) the heavy chain comprises the amino acid sequence of SEQ ID NO: 121, the light chain comprises the amino acid sequence of SEQ ID NO: 108, and the third polypeptide chain comprises the amino acid sequence of SEQ ID NO: 124;
(q) the heavy chain comprises the amino acid sequence of SEQ ID NO: 131, the light chain comprises the amino acid sequence of SEQ ID NO: 127, and the third polypeptide chain comprises the amino acid sequence of SEQ ID NO: 124; or
(r) the heavy chain comprises the amino acid sequence of SEQ ID NO: 120, the light chain comprises the amino acid sequence of SEQ ID NO: 127, and the third polypeptide chain comprises the amino acid sequence of SEQ ID NO: 124.

Further provided herein are proteins LB201, LB202, LB203, LB204, LB205, LB206, LB207, LB208, LB209, LB210, LB211, LB212, LB213, LB214, LB215, LB216, LB217, LB218, LB219, LB220, LB221, LB222, LB223, LB224, LB225, LB226, LB227, LB228, LB229, LB230, LB231, LB232, LB233, LB234, LB235, LB236, LB237, LB238, LB239, LB240, LB241, LB242, LB243, and LB244. The sequences of the polypeptide chains of these proteins are provided in Table 9.

TABLE 9
Chain sequence combinations used to form illustrative LB proteins as
described in FIG. 1 and based on sequences specified in Tables 7 and 8

| | | | Heavy Chain |
|---|---|---|---|
| Name | Heavy Chain Construct | Light Chain Construct | Construct 2 |
| LB201 | 5HH (SEQ ID NO: 122) | 6LL-F (SEQ ID NO: 114) | Fc-stump (SEQ ID |
| | | | NO: 124) |
| LB202 | 6HH (SEQ ID NO: 123) | 7LL-F (SEQ ID NO: 115) | Fc-stump (SEQ ID |
| | | | NO: 124) |
| LB203 | 1HH (SEQ ID NO: 116) | 1LL-F (SEQ ID NO: 108) | Fc-stump (SEQ ID |
| | | | NO: 124) |
| LB204 | 1HHX (SEQ ID NO: 117) | 1LLX-F (SEQ ID NO: 109) | Fc-stump (SEQ ID |
| | | | NO: 124) |
| LB205 | 2HH (SEQ ID NO: 118) | 1LL-F (SEQ ID NO: 108) | Fc-stump (SEQ ID |
| | | | NO: 124) |
| LB206 | 2HHX (SEQ ID NO: 119) | 1LLX-F (SEQ ID NO: 109) | Fc-stump (SEQ ID |
| | | | NO: 124) |
| LB207 | 3HH (SEQ ID NO: 120) | 1LL-F (SEQ ID NO: 108) | Fc-stump (SEQ ID |
| | | | NO: 124) |
| LB208 | 1HH (SEQ ID NO: 116) | 2LL-F (SEQ ID NO: 110) | Fc-stump (SEQ ID |
| | | | NO: 124) |
| LB209 | 2HH (SEQ ID NO: 118) | 2LL-F (SEQ ID NO: 110) | Fc-stump (SEQ ID |
| | | | NO: 124) |
| LB210 | 3HH (SEQ ID NO: 120) | 2LL-F (SEQ ID NO: 110) | Fc-stump (SEQ ID |
| | | | NO: 124) |
| LB211 | 1HH (SEQ ID NO: 116) | 3LL-F (SEQ ID NO: 111) | Fc-stump (SEQ ID |
| | | | NO: 124) |
| LB212 | 2HH (SEQ ID NO: 118) | 3LL-F (SEQ ID NO: 111) | Fc-stump (SEQ ID |
| | | | NO: 124) |
| LB213 | 3HH (SEQ ID NO: 120) | 3LL-F (SEQ ID NO: 111) | Fc-stump (SEQ ID |
| | | | NO: 124) |
| LB214 | 1HH (SEQ ID NO: 116) | 4LL-F (SEQ ID NO: 112) | Fc-stump (SEQ ID |
| | | | NO: 124) |
| LB215 | 1HH (SEQ ID NO: 116) | 5LL-F (SEQ ID NO: 113) | Fc-stump (SEQ ID |
| | | | NO: 124) |
| LB216 | 4HH (SEQ ID NO: 121) | 1LL-F (SEQ ID NO: 108) | Fc-stump (SEQ ID |
| | | | NO: 124) |
| LB217 | 2HHX (SEQ ID NO: 119) | 1LLRX-F (SEQ ID NO: | Fc-stump (SEQ ID |
| | | 126) | NO: 124) |
| LB218 | 3HH (SEQ ID NO: 120) | 3LLR-F (SEQ ID NO: 127) | Fc-stump (SEQ ID |
| | | | NO: 124) |
| LB219 | 2HHRX-F (SEQ ID NO: | 1LLX-F (SEQ ID NO: 109) | Fc-stump (SEQ ID |
| | 130) | | NO: 124) |
| LB220 | 3HHR-F (SEQ ID NO: 131) | 3LLR-F (SEQ ID NO: 127) | Fc-stump (SEQ ID |
| | | | NO: 124) |
| LB221 | 2LHX (SEQ ID NO: 143) | 1HLX-F (SEQ ID NO: 142) | Fc-stump (SEQ ID |
| | | | NO: 124) |
| LB222 | 3LH (SEQ ID NO: 145) | 3HL-F (SEQ ID NO: 144) | Fc-stump (SEQ ID |
| | | | NO: 124) |
| LB223 | 2LHX (SEQ ID NO: 143) | 1HLX-R (SEQ ID NO: 146) | Fc-stump (SEQ ID |
| | | | NO: 124) |
| LB224 | 3LH (SEQ ID NO: 145) | 3HL-FR (SEQ ID NO: 148) | Fc-stump (SEQ ID |
| | | | NO: 124) |
| LB225 | 2LHX-R (SEQ ID NO: 151) | 1HLX (SEQ ID NO: 150) | Fc-stump (SEQ ID |
| | | | NO: 124) |
| LB226 | 3LHRX-F (SEQ ID NO: | 3HL (SEQ ID NO: 152) | Fc-stump (SEQ ID |
| | 153) | | NO: 124) |
| LB227 | 2HH (SEQ ID NO: 118) | 1LLX-F (SEQ ID NO: 109) | Fc-stump (SEQ ID |
| | | | NO: 124) |
| LB228 | 2HHX (SEQ ID NO: 119) | 1LL-F (SEQ ID NO:108) | Fc-stump (SEQ ID |
| | | | NO: 124) |
| LB229 | 3HH (SEQ ID NO: 120) | 3LLX-F (SEQ ID NO: 53) | Fc-stump (SEQ ID |
| | | | NO: 124) |
| LB230 | 3HHX (SEQ ID NO: 147) | 3LL-F (SEQ ID NO: 111) | Fc-stump (SEQ ID |
| | | | NO: 124) |
| LB231 | 3HHX (SEQ ID NO: 147) | 3LLX-F (SEQ ID NO: 53) | Fc-stump (SEQ ID |
| | | | NO: 124) |
| LB232 | B-2HH (SEQ ID NO: 137) | B-1LL-F (SEQ ID NO: 133) | |
| LB233 | B-2HH (SEQ ID NO: 137) | B-1LLX-F (SEQ ID NO: | |
| | | 134) | |
| LB234 | B-2HHX (SEQ ID NO: 138) | B-1LL-F (SEQ ID NO: 133) | |
| LB235 | B-2HHX (SEQ ID NO: 138) | B-1LLX-F (SEQ ID NO: | |
| | | 134) | |
| LB236 | B-3HH (SEQ ID NO: 139) | B-3LL-F (SEQ ID NO: 135) | |
| LB237 | B-3HH (SEQ ID NO: 139) | B-3LLX-F (SEQ ID NO: | |
| | | 136) | |
| LB238 | B-3HHX (SEQ ID NO: 140) | B-3LL-F (SEQ ID NO: 135) | |
| LB239 | B-3HHX (SEQ ID NO: 140) | B-3LLX-F (SEQ ID NO: | |
| | | 136) | |
| LB240 | 2HH (SEQ ID NO: 118) | 1LL-F (SEQ ID NO: 108) | Fc-stump (SEQ ID |
| | | | NO: 124) |
| LB241 | 3HHR-F (SEQ ID NO: 131) | 3LL-F (SEQ ID NO: 111) | Fc-stump (SEQ ID |
| | | | NO: 124) |
| LB242 | 2HH (SEQ ID NO: 118) | 1LLR-F (SEQ ID NO: 125) | Fc-stump (SEQ ID |
| | | | NO: 124) |
| LB243 | 2HHR-F (SEQ ID NO: 129) | 1LL-F (SEQ ID NO: 108) | Fc-stump (SEQ ID |
| | | | NO: 124) |
| LB244 | 2HHX-HLE (SEQ ID NO: | 1LLX-F (SEQ ID NO: 109) | |
| | 141) | | |

Also provided herein is a protein comprising a heavy chain and a light chain, wherein the heavy chain comprises an amino acid sequence provided herein, with 1, 2 or 3 conservative amino acid substitutions; and wherein the light chain comprises an amino acid sequence provided herein, with 1, 2 or 3 conservative amino acid substitutions. In some embodiments, conservative amino acid substitutions are made only in the FR sequences and not in the CDR sequences. In some embodiments, conservative amino acid substitutions are not made in the first linker or the second linker sequences.

In some embodiments, a protein provided herein comprises an immunoglobulin heavy chain constant region at the C-terminus of the heavy chain. In some embodiments, a protein provided herein comprises an immunoglobulin heavy chain constant region at the C-termini of both the first polypeptide chain and the second polypeptide chain. In some embodiments, the immunoglobulin heavy chain constant region is IgG, IgE, IgM, IgD, IgA or IgY. In some embodiments, the immunoglobulin heavy chain constant region is IgG1, IgG2, IgG3, IgG4, IgA1 or IgA2. In some embodiments, the immunoglobulin heavy chain constant region is IgG1. In some embodiments, the immunoglobulin heavy chain constant region is immunologically inert. In some embodiments, the immunoglobulin heavy chain constant region comprises one or more mutations to reduce or prevent FcγR binding, antibody-dependent cell-mediated cytotoxicity (ADCC) activity, antibody-dependent cellular phagocytosis (ADCP), and/or complement-dependent cytotoxicity (CDC) activity. In some embodiments, the immunoglobulin heavy chain constant region is a wild-type human IgG1 constant region, a wild-type human IgG2 constant region, a wild-type human IgG4 constant region, a human IgG1 constant region comprising the amino acid substitutions L234A, L235A and G237A, a human IgG1 constant region comprising the amino acid substitutions L234A, L235A, G237A and P331S or a human IgG4 constant region comprising the amino acid substitution S228P, wherein numbering is according to the EU index as in Kdbat. In some embodiments, a position of an amino acid residue in a constant region of an immunoglobulin molecule is numbered according to the EU index as in Kdbat (Ward et al., 1995 Therap. Immunol. 2:77-94).

When proteins provided herein comprise CH2 and CH3 regions on two polypeptide chains, the CH2 or CH3 regions may comprise sites to aid with pairing of the two polypeptide chains. Any suitable Fc heterodimerization technology may be used for pairing the polypeptide chains. In some embodiments, N-linked glycosylation sites are included in the CH2 regions. In some embodiments, Knobs and Holes mutations are included in the CH3 regions.

In some embodiments, a protein provided herein may comprise an immunoglobulin light chain constant region that is a kappa light chain. In some embodiments a kappa light chain comprises SEQ ID NO: 15.

In some embodiments, a protein provided herein may comprise an immunoglobulin light chain constant region that is a lambda light chain.

In some embodiments, a protein provided herein may comprise an immunoglobulin heavy chain constant region comprising an amino acid sequence of an Fc region of human IgG4, human IgG4 (S228P), human IgG2, human IgG1, human IgG1 effector null. For example, the human IgG4 (S228P) Fc region comprises the following substitution compared to the wild-type human IgG4 Fc region: S228P. For example, the human IgG1 effector null Fc region comprises the following substitutions compared to the wild-type human IgG1 Fc region: L234A, L235A and G237A. In some embodiments, a protein may comprise an immunoglobulin heavy chain constant region comprising the amino acid sequence of any one of SEQ ID NO: 13, SEQ ID NO: 14, SEQ ID NO: 16, SEQ ID NO: 17, SEQ ID NO: 18, and SEQ ID NO: 19. In some embodiments, a protein may comprise an immunoglobulin heavy chain constant region comprising an amino acid sequence that is at least 95%, 96%, 97%, 98% or 99% identical to the amino acid sequence of any one of SEQ ID NO: 13, SEQ ID NO: 14, SEQ ID NO: 16, SEQ ID NO: 17, SEQ ID NO: 18, and SEQ ID NO: 19.

In some embodiments, a protein may comprise a hinge (e.g., a Fc hinge) that is a wild-type human IgG1 hinge, wild-type human IgG2 hinge, wild-type human IgG3 hinge, or wild-type human IgG4 hinge. In some embodiments, a protein may comprise an amino acid sequence that is at least 95%, 96%, 97%, 98% or 99% identical to the amino acid sequence of a wild-type human IgG1 hinge, wild-type human IgG2 hinge, wild-type human IgG3 hinge, or wild-type human IgG4 hinge. In some embodiments, a protein may comprise a hinge comprising an amino acid sequence that is at least 95%, 96%, 97%, 98% or 99% identical to the amino acid sequence of the hinge sequence in any one of SEQ ID NO: 13, SEQ ID NO: 14, SEQ ID NO: 16, SEQ ID NO: 17, SEQ ID NO: 18, and SEQ ID NO: 19.

Provided herein is an immunoconjugate comprising a protein disclosed herein linked to a therapeutic agent. In some embodiments, the therapeutic agent is a cytotoxin, a radioisotope, a chemotherapeutic agent, an immunomodulatory agent, a cytostatic enzyme, a cytolytic enzyme, a therapeutic nucleic acid, an anti-angiogenic agent, an anti-proliferative agent, or a pro-apoptotic agent.

Examples of suitable therapeutic agents include, but are not limited to, immunomodulatory agents, cytotoxins, radioisotopes, chemotherapeutic agents, anti-angiogenic agents, antiproliferative agents, pro-apoptotic agents, and cytostatic and cytolytic enzymes (for example, RNAses). Further therapeutic agents include a therapeutic nucleic acid, such as a gene encoding an immunomodulatory agent, an anti-angiogenic agent, an anti-proliferative agent, or a pro-apoptotic agent. These drug descriptors are not mutually exclusive, and thus a therapeutic agent may be described using one or more of the above terms.

Examples of suitable therapeutic agents for use in immunoconjugates include, but are not limited to, JAK kinase inhibitors, taxanes, maytansines, CC-1065 and the duocarmycins, the calicheamicins and other enediynes, and the auristatins. Other examples include the anti-folates, vinca alkaloids, and the anthracyclines. Plant toxins, other bioactive proteins, enzymes (i.e., ADEPT), radioisotopes, photosensitizers may also be used in immunoconjugates. In addition, conjugates can be made using secondary carriers as the cytotoxic agent, such as liposomes or polymers, Suitable cytotoxins include an agent that inhibits or prevents the function of cells and/or results in destruction of cells. Representative cytotoxins include antibiotics, inhibitors of tubulin polymerization, alkylating agents that bind to and disrupt DNA, and agents that disrupt protein synthesis or the function of essential cellular proteins such as protein kinases, phosphatases, topoisomerases, enzymes, and cyclins.

Representative cytotoxins include, but are not limited to, doxorubicin, daunorubicin, idarubicin, aclarubicin, zorubicin, mitoxantrone, epirubicin, carubicin, nogalamycin, menogaril, pitarubicin, valrubicin, cytarabine, gemcitabine, trifluridine, ancitabine, enocitabine, azacitidine, doxifluhdine, pentostatin, broxuhdine, capecitabine, cladhbine, decitabine, floxuhdine, fludarabine, gougerotin, puromycin, tegafur, tiazofuhn, adhamycin, cisplatin, carboplatin, cyclophosphamide, dacarbazine, vinblastine, vincristine, mitoxantrone, bleomycin, mechlorethamine, prednisone, procarbazine, methotrexate, flurouracils, etoposide, taxol, taxol analogs, platins such as cis-platin and carbo-platin, mitomycin, thiotepa, taxanes, vincristine, daunorubicin, epirubicin, actinomycin, authramycin, azaserines, bleomycins, tamoxifen, idarubicin, dolastatins/auristatins, hemiasterlins, esperamicins and maytansinoids.

Suitable immunomodulatory agents include anti-hormones that block hormone action on tumors and immunosuppressive agents that suppress cytokine production, down-regulate self-antigen expression, or mask MHC antigens.

### Pharmaceutical Compositions

The activatable proteins provided herein (also referred to herein as “active compounds”) can be incorporated into pharmaceutical compositions suitable for administration. Such compositions typically comprise a protein (or an immunoconjugate comprising said protein), and a pharmaceutically acceptable carrier.

In some embodiments, such compositions typically comprise a protein (or an immunoconjugate comprising said protein), and a pharmaceutically acceptable carrier, diluent or excipient. Such materials should be non-toxic and should not interfere with the efficacy of the protein. The precise nature of the carrier or other material will depend on the route of administration, which may be by injection, bolus, infusion, or any other suitable route, as discussed below.

As used herein, the term “pharmaceutically acceptable” refers to molecular entities and compositions that do not generally produce allergic or other serious adverse reactions when administered using routes well known in the art. Molecular entities and compositions approved by a regulatory agency of the U.S. federal or state government or listed in the U.S. Pharmacopeia or other generally recognized pharmacopeia for use in animals, and more particularly in humans are considered to be “pharmaceutically acceptable.” As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Some examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions. A pharmaceutically acceptable carrier, diluent or excipient may be a compound or a combination of compounds that does not provoke secondary reactions and that allows, for example, facilitation of the administration of the protein, an increase in its lifespan and/or in its efficacy in the body or an increase in its solubility in solution.

A pharmaceutical composition disclosed herein may be formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, NJ) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primojel®, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

For administration by inhalation, the compounds may be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical agents can also be prepared in the form of suppositories (e.g., with conventional suppository bases such as cocoa butter and other glycerides) or retention enemas for rectal delivery.

In some embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

In some embodiments, the protein may be provided in a lyophilized form for reconstitution prior to administration. For example, lyophilized antibody molecules may be reconstituted in sterile water and mixed with saline prior to administration to an individual.

The pharmaceutical compositions provided herein can be included in a container, pack, or dispenser together with instructions for administration.

### Nucleic Acid Molecules, Vectors, Host Cells and Methods of Producing Proteins

Provided herein is a nucleic acid molecule (e.g., an isolated nucleic acid molecule) encoding an amino acid sequence of a protein disclosed herein (or an amino acid sequence of a (i) VH domain, (ii) a VL domain, or (iii) both a VH domain and a VL domain of a protein). Further provided herein is a nucleic acid molecule (e.g., an isolated nucleic acid molecule) encoding (i) a heavy chain, (ii) a light chain, or (iii) both a heavy chain and a light chain of a protein disclosed herein. Further provided herein is a nucleic acid molecule (e.g., an isolated nucleic acid molecule) encoding (i) a first polypeptide chain, (ii) a second polypeptide chain, or (iii) both a first polypeptide chain and a second polypeptide chain of a protein disclosed herein. In some embodiments, a nucleic acid molecule further encodes a third polypeptide chain (e.g., a third polypeptide chain comprising a hinge, a CH2 domain, and a CH3 domain).

In some embodiments, a nucleic acid molecule encoding a VH domain, a VL domain, a heavy chain, a light chain, a first polypeptide chain, or a second polypeptide chain comprises a signal sequence (or encodes a leader peptide). In some embodiments, a nucleic acid molecule encoding a VH domain, a VL domain, a heavy chain, a light chain, a first polypeptide chain, or a second polypeptide chain does not comprise a signal sequence (or does not encode a leader peptide).

Also provided herein is an expression vector comprising a nucleic acid molecule described herein. In certain vectors, a nucleic acid molecule is operatively linked to one or more regulatory sequences suitable for expression of the nucleic acid segment in a host cell. In some cases, an expression vector comprises sequences that mediate replication and comprises one or more selectable markers. As used herein, “vector” means a construct that is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

Provided herein is a recombinant host cell comprising an expression vector or a nucleic acid molecule disclosed herein. A “host cell” includes an individual cell, a cell line or cell culture that can be or has been a recipient for vector(s) for incorporation of polynucleotide inserts. Host cells include progeny of a single host cell. The progeny may not necessarily be completely identical (in morphology or in genomic DNA complement) to the original parent cell due to natural, accidental, or deliberate mutation. An expression vector can be transfected into a host cell by standard techniques. Non-limiting examples include electroporation, calcium-phosphate precipitation, DEAE-dextran transfection and the like. In some embodiments, a recombinant host cell comprises a single vector or a single nucleic acid molecule encoding both a heavy chain and a light chain of a protein disclosed herein. In some embodiments, a recombinant host cell comprises (i) a first vector or a first nucleic acid molecule encoding a heavy chain of a protein disclosed herein and (ii) a second vector or a second nucleic acid molecule encoding a light chain of a protein disclosed herein.

Protein molecules of the invention, or portions thereof, can be produced using techniques well known in the art, for example, recombinant technologies, phage display technologies, synthetic technologies, computational technologies or combinations of such technologies or other technologies readily known in the art.

Further provided herein is a method for producing a protein disclosed herein, the method comprising: culturing a recombinant host cell comprising an expression vector described herein under conditions whereby the nucleic acid segment is expressed, thereby producing the protein. The protein may then be isolated from the host cell or culture. Provided herein is a method of producing a protein, the method comprising: culturing a recombinant host cell comprising an expression vector disclosed herein under conditions whereby the nucleic acid molecule is expressed, thereby producing the protein; and isolating the protein from the host cell or culture.

Proteins disclosed herein can be produced by any of a variety of methods known to those skilled in the art. In certain embodiments, proteins disclosed herein can be produced recombinantly. For example, nucleic acid sequences encoding one or more of the heavy chains or light chains provided herein, or portions thereof, may be introduced into a bacterial cell (e.g., E. coli, B. subtilis) or a eukaryotic cell (e.g., a yeast such as S. cerevisiae, or a mammalian cell such as a CHO cell line, various Cos cell lines, a HeLa cell, a HEK293 cell, various myeloma cell lines, or a transformed B-cell or hybridoma), or into an in vitro translation system, and the translated polypeptide may be isolated. In some embodiments, light chain proteins and heavy chain proteins are produced in a cell with a signal sequence that is removed upon production of a mature protein disclosed herein.

Those skilled in the art will be able to determine whether a protein comprising a given polypeptide sequence binds to PD-L1 protein and/or CD3 protein using standard methodologies, for example, Western blots, ELISA, and the like.

### Medical Uses of Activatable Proteins

Provided herein are methods and uses of activatable proteins, immunoconjugates, and pharmaceutical compositions disclosed herein for providing a therapeutic benefit to a subject with cancer.

An activatable protein, immunoconjugate, or pharmaceutical composition disclosed herein may be used in a method of treatment of the human or animal body, including prophylactic or preventative treatment (e.g., treatment before the onset of a condition in a subject to reduce the risk of the condition occurring in the subject; delay its onset; or reduce its severity after onset). The method of treatment may comprise administering the protein, immunoconjugate, or pharmaceutical composition to a subject in need thereof.

Provided herein is a method for enhancing an anti-cancer immune response in a subject, the method comprising administering to the subject a therapeutically effective amount of a protein, an immunoconjugate, or a pharmaceutical composition disclosed herein. In some embodiments, an anti-cancer immune response is a T cell response. In some embodiments, an anti-cancer immune response is a complement response.

Provided herein is a method for treating cancer in a subject, the method comprising administering to the subject a therapeutically effective amount of a protein, an immunoconjugate, or a pharmaceutical composition disclosed herein.

Provided herein is a method for ameliorating a symptom of cancer in a subject, the method comprising administering to the subject a therapeutically effective amount of a protein, immunoconjugate, or a pharmaceutical composition disclosed herein.

Provided herein is a method for reducing the size of a tumor in a subject, the method comprising administering to the subject a therapeutically effective amount of a protein, an immunoconjugate, or a pharmaceutical composition disclosed herein.

Provided herein is a method for inhibiting the growth of a tumor in a subject, the method comprising administering to the subject a therapeutically effective amount of a protein, an immunoconjugate, or a pharmaceutical composition disclosed herein.

Provided herein is a protein, an immunoconjugate, or a pharmaceutical composition disclosed herein, for use in enhancing an anti-cancer immune response in a subject.

Provided herein is a protein, an immunoconjugate, or a pharmaceutical composition disclosed herein, for use in treating cancer in a subject.

Provided herein is a protein, an immunoconjugate, or a pharmaceutical composition disclosed herein, for use in ameliorating a symptom of cancer in a subject.

In some embodiments, the cancer is gastrointestinal stromal cancer (GIST), pancreatic cancer, skin cancer, melanoma, breast cancer, lung cancer, bronchial cancer, colorectal cancer, prostate cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of the oral cavity or pharynx, liver cancer, kidney cancer, renal cell carcinoma, testicular cancer, biliary tract cancer, small bowel cancer, appendix cancer, salivary gland cancer, thyroid cancer, adrenal gland cancer, osteosarcoma, chondrosarcoma, or cancer of hematological tissues.

In some embodiments, the cancer is a solid tumor. In some embodiments, the cancer is a hematological cancer.

In some embodiments, a cancer of hematological tissues is a lymphoma. In some embodiments, the cancer is mantle cell lymphoma, acute lymphoblastic leukemia, chronic lymphocytic leukemia, Non-Hodgkin's lymphoma, Hodgkin's lymphoma, acute myeloid leukemia (AML), B-lymphoid leukemia, blastic plasmocytoid dendritic neoplasm (BPDCN), or hairy cell leukemia.

As used herein, the term “effective amount” or “therapeutically effective amount” refers to the amount of a pharmaceutical agent, e.g., a protein, an immunoconjugate, or a pharmaceutical composition disclosed herein, which is sufficient to reduce or ameliorate the severity and/or duration of a cancer, or one or more symptoms thereof, prevent the advancement of a disease, cause regression of a disease, prevent the recurrence, development, onset or progression of one or more symptoms associated with a disease, or enhance or improve the prophylactic or therapeutic effect(s) of another related therapy (e.g., prophylactic or therapeutic agent) for a cancer.

The actual amount administered, and rate and time-course of administration, will depend on the nature and severity of what is being treated, the particular mammal being treated, the clinical condition of the individual patient, the cause of the disorder, the site of delivery of the composition, the method of administration, the scheduling of administration and other factors known to medical practitioners. Prescription of treatment, e.g., decisions on dosage etc., is within the responsibility of general practitioners and other medical doctors and may depend on the severity of the symptoms and/or progression of a disease being treated. Appropriate doses of antibody-based protein molecules are well known in the art (Ledermann J. A. et al., 1991, Int. J Cancer 47: 659-664; Bagshawe K. D. et al., 1991, Antibody, Immunoconjugates and Radiopharmaceuticals 4: 915-922). Specific dosages may be indicated herein or in the Physician's Desk Reference (2003) as appropriate for the type of medicament being administered may be used. A therapeutically effective amount or suitable dose of an antibody-based protein molecule may be determined by comparing its in vitro activity and in vivo activity in an animal model. Methods for extrapolation of effective dosages in mice and other test animals to humans are known. The precise dose will depend upon a number of factors, including whether the antibody-based protein is for prevention or for treatment, the size and location of the area to be treated, the precise nature of the antibody-based protein, and the nature of any detectable label or other molecule attached to the antibody-based protein.

A typical protein dose will be in the range 100 μg to 1 g for systemic applications, and 1 μg to 1 mg for intradermal injection. An initial higher loading dose, followed by one or more lower doses, may be administered. In some embodiments, the protein is an IgG1 or IgG4 isotype. A dose for a single treatment of an adult subject may be proportionally adjusted for children and infants. Treatments may be repeated at daily, twice-weekly, weekly or monthly intervals, at the discretion of the physician. The treatment schedule for a subject may be dependent on the pharmacokinetic and pharmacodynamic properties of the protein composition, the route of administration and the nature of the condition being treated.

Treatment may be periodic, and the period between administrations may be about two weeks or more, e.g., about three weeks or more, about four weeks or more, about once a month or more, about five weeks or more, or about six weeks or more. For example, treatment may be every two to four weeks or every four to eight weeks. Treatment may be given before, and/or after surgery, and/or may be administered or applied directly at the anatomical site of surgical treatment or invasive procedure. Suitable formulations and routes of administration are described above.

In some embodiments, a protein, an immunoconjugate, or a pharmaceutical composition disclosed herein may be administered as a sub-cutaneous injection. Sub-cutaneous injections may be administered using an auto-injector, for example for long term prophylaxis/treatment.

In some embodiments, the therapeutic effect of a protein, an immunoconjugate, or a pharmaceutical composition disclosed herein may persist for several half-lives, depending on the dose. For example, the therapeutic effect of a single dose of a protein, an immunoconjugate, or a pharmaceutical composition disclosed herein may persist in a subject for 1 month or more, 2 months or more, 3 months or more, 4 months or more, 5 months or more, or 6 months or more.

In some embodiments, a subject may be treated with a protein, an immunoconjugate, or a pharmaceutical composition disclosed herein and an additional therapeutic agent or therapy that is used to treat a cancer or a symptom or complication of a cancer. The protein, immunoconjugate, or pharmaceutical composition disclosed herein and the additional therapeutic agent or therapy may be administered simultaneously or sequentially.

In some embodiments, a subject is a mammal, a human, a non-human primate, a pig, a horse, a cow, a dog, a cat, a guinea pig, a mouse or a rat. In some embodiments, a subject is an adult human. In some embodiments, a subject is a pediatric human. In some embodiments, a human subject is 16 years of age or older. In some embodiments, a human subject is 18 years of age or older. In some embodiments, a human subject is less than 16 years of age. In some embodiments, a human subject is less than 18 years of age.

Further provided herein is a protein, an immunoconjugate, or a pharmaceutical composition disclosed herein, for use in the treatment of a disease or a disorder.

Provided herein is a protein, an immunoconjugate, or a pharmaceutical composition disclosed herein, for use as a medicament.

### Definitions

Unless otherwise noted, the terms used herein have definitions as ordinarily used in the art. Some terms are defined below, and additional definitions can be found within the rest of the detailed description.

As used herein and unless otherwise stated, the terms “hinge linker”, “linker”, “hinge”, “first linker”, “second linker”, “lower hinge linker” (“LHL”), “inter-Fab linker”, and derivations thereof, in plural or singular form, refer to a sequence, for example derived from an immunoglobulin hinge region, that can link two polypeptides, for example polypeptides of different Fab regions, and is separate from any hinge sequence in an immunoglobulin hinge region that may be part of a protein of the present invention.

LB protein refers to LockBody proteins of the instant invention with a structure as defined in FIG. 1.

The term “a” or “an” refers to one or more of that entity, i.e., can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein, the term “sequence identity” refers to the extent to which two optimally aligned polynucleotides or polypeptide sequences are invariant throughout a window of alignment of residues, e.g., nucleotides or amino acids. An “identity fraction” for aligned segments of a test sequence and a reference sequence is the number of identical residues which are shared by the two aligned sequences divided by the total number of residues in the reference sequence segment, i.e., the entire reference sequence or a smaller defined part of the reference sequence. “Percent identity” is the identity fraction times 100. Percentage identity can be calculated using the alignment program Clustal Omega, available at ebi.ac.uk/Tools/msa/clustalo using default parameters. See, Sievers et al., “Fast, scalable generation of high-quality protein multiple sequence alignments using Clustal Omega” (2011 October 11) Molecular Systems Biology 7:539. For the purposes of calculating identity to the sequence, extensions, such as tags, are not included.

As used herein, the term “HCDR” refers to a heavy chain complementarity determining region. As used herein, the term “LCDR” refers to a light chain complementarity determining region.

The terms “amino-terminal”, “N-terminus”, “carboxyl-terminal”, and “C-terminus” are used herein to denote positions within polypeptide chains. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl-terminus of the reference sequence but is not necessarily at the carboxyl-terminus of the complete polypeptide.

As used herein, the term “conservative substitution” refers to replacement of an amino acid with another amino acid which does not significantly deleteriously change the functional activity. A preferred example of a “conservative substitution” is the replacement of one amino acid with another amino acid which has a value ≥0 in the following BLOSUM 62 substitution matrix (see Henikoff & Henikoff, 1992, PNAS 89: 10915-10919):

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | −1 | −2 | −2 | 0 | −1 | −1 | 0 | −2 | −1 | −1 | −1 | −1 | −2 | −1 | 1 | 0 | −3 | −2 | 0 |
| R | −1 | 5 | 0 | −2 | −3 | 1 | 0 | −2 | 0 | −3 | −2 | 2 | −1 | −3 | −2 | −1 | −1 | −3 | −2 | −3 |
| N | −2 | 0 | 6 | 1 | −3 | 0 | 0 | 0 | 1 | −3 | −3 | 0 | −2 | −3 | −2 | 1 | 0 | −4 | −2 | −3 |
| D | −2 | −2 | 1 | 6 | −3 | 0 | 2 | −1 | −1 | −3 | −4 | −1 | −3 | −3 | −1 | 0 | −1 | −4 | −3 | −3 |
| C | 0 | −3 | −3 | −3 | 9 | −3 | −4 | −3 | −3 | −1 | −1 | −3 | −1 | −2 | −3 | −1 | −1 | −2 | −2 | −1 |
| Q | −1 | 1 | 0 | 0 | −3 | 5 | 2 | −2 | 0 | −3 | −2 | 1 | 0 | −3 | −1 | 0 | −1 | −2 | −1 | −2 |
| E | −1 | 0 | 0 | 2 | −4 | 2 | 5 | −2 | 0 | −3 | −3 | 1 | −2 | −3 | −1 | 0 | −1 | −3 | −2 | −2 |
| G | 0 | −2 | 0 | −1 | −3 | −2 | −2 | 6 | −2 | −4 | −4 | −2 | −3 | −3 | −2 | 0 | −2 | −2 | −3 | −3 |
| H | −2 | 0 | 1 | −1 | −3 | 0 | 0 | −2 | 8 | −3 | −3 | −1 | −2 | −1 | −2 | −1 | −2 | −2 | 2 | −3 |
| I | −1 | −3 | −3 | −3 | −1 | −3 | −3 | −4 | −3 | 4 | 2 | −3 | 1 | 0 | −3 | −2 | −1 | −3 | −1 | 3 |
| L | −1 | −2 | −3 | −4 | −1 | −2 | −3 | −4 | −3 | 2 | 4 | −2 | 2 | 0 | −3 | −2 | −1 | −2 | −1 | 1 |
| K | −1 | 2 | 0 | −1 | −3 | 1 | 1 | −2 | −1 | −3 | −2 | 5 | −1 | −3 | −1 | 0 | −1 | −3 | −2 | −2 |
| M | −1 | −1 | −2 | −3 | −1 | 0 | −2 | −3 | −2 | 1 | 2 | −1 | 5 | 0 | −2 | −1 | −1 | −1 | −1 | 1 |
| F | −2 | −3 | −3 | −3 | −2 | −3 | −3 | −3 | −1 | 0 | 0 | −3 | 0 | 6 | −4 | −2 | −2 | 1 | 3 | −1 |
| P | −1 | −2 | −2 | −1 | −3 | −1 | −1 | −2 | −2 | −3 | −3 | −1 | −2 | −4 | 7 | −1 | −1 | −4 | −3 | −2 |
| S | 1 | −1 | 1 | 0 | −1 | 0 | 0 | 0 | −1 | −2 | −2 | 0 | −1 | −2 | −1 | 4 | 1 | −3 | −2 | −2 |
| T | 0 | −1 | 0 | −1 | −1 | −1 | −1 | −2 | −2 | −1 | −1 | −1 | −1 | −2 | −1 | 1 | 5 | −2 | −2 | 0 |
| W | −3 | −3 | −4 | −4 | −2 | −2 | −3 | −2 | −2 | −3 | −2 | −3 | −1 | 1 | −4 | −3 | −2 | 11 | 2 | −3 |
| Y | −2 | −2 | −2 | −3 | −2 | −1 | −2 | −3 | 2 | −1 | −1 | −2 | −1 | 3 | −3 | −2 | −2 | 2 | 7 | −1 |
| V | 0 | −3 | −3 | −3 | −1 | −2 | −2 | −3 | −3 | 3 | 1 | −2 | 1 | −1 | −2 | −2 | 0 | −3 | −1 | 4. |

The term “immunoconjugate” refer to a protein of the disclosure that is conjugated to a cytotoxic, a cytostatic and/or a therapeutic agent.

The term “isolated molecule” (where the molecule is, for example, a protein, a nucleic acid, a polynucleotide, or an antibody) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

The terms “inhibit”, “block”, or “neutralize”, as used herein with respect to bioactivity of a protein disclosed herein means the ability of the protein to substantially antagonize, prohibit, prevent, restrain, slow, disrupt, eliminate, stop, reduce or reverse for example progression, strength, or severity of that which is being inhibited including, but not limited to, the binding of PD-L1 to PD-1, or the binding of CD3 to the T-cell receptor (TCR).

As used herein, the terms “treat,” “treating” or “treatment of” (and grammatical variations thereof) mean that the severity of the subject's condition is reduced, at least partially improved or stabilized and/or that some alleviation, mitigation, decrease or stabilization in at least one clinical symptom is achieved and/or there is a delay in the progression of the disease or disorder.

PD-L1 is also known as programmed cell death ligand 1, CD274, B7-H, B7H1, PDCDILI, PDCDILGI, PDL1, and hPD-L1. Illustrative PD-L1 amino acid sequences are provided as SEQ ID NO: 35 and SEQ ID NO: 36.

CD3 is also known as cluster of differentiation 3. CD3 is a multimeric protein complex. CD3 is composed of four distinct polypeptide chains; epsilon (ε), gamma (γ), delta (δ) and zeta (ξ)

As used herein, the terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the compositions and/or methods described herein. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the compositions and/or methods described herein.

As used herein, a “therapeutically effective amount” is the amount of a protein or a pharmaceutical composition provided herein that is effective to treat a disease or disorder in a subject or to ameliorate a sign or symptom thereof. The “therapeutically effective amount” may vary depending, for example, on the disease and/or symptoms of the disease, severity of the disease and/or symptoms of the disease or disorder, the age, weight, and/or health of the patient to be treated, and the judgment of the prescribing physician.

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

The section headings used herein are for organizational purposes only and are not to be construed as limiting the subject matter described.

The disclosure will be further clarified by the following example, which is intended to be purely exemplary of the disclosure and in no way limiting.

### Example

### Generation of Optimized Conditionally Active Therapeutic Proteins

### INTRODUCTION

In this example, we successfully generated certain antibodies of the instant invention, wherein the antibodies exhibit restricted binding toward a CD3 epitope until at least one lower hinge linker is targeted and cleaved by one or more proteases, e.g., MMP, which are often found to be highly active in the TME.

### Materials and Methods

### Cloning, Transient Expression, Purification and Characterization of Proteins

Polypeptide-encoding DNA sequences were cloned via restriction-ligation cloning into separate human IgG1 heavy and light-chain constant region-encoding expression cassettes in separate plasmid vectors, to create activatable constructs for expression. Proteins were expressed in CHO cells and purified from culture supernatants via a combination of Protein A affinity chromatography (ProA), Ion Exchange Chromatography (IEX) and/or Size Exclusion Chromatography (SEC). Purified proteins were characterized by SEC, SDS-PAGE and mass spectrometry.

### Metalloprotease Digestion

Protein constructs were incubated for time increments between 0 and 24 hours at 37° C. with human Matrix Metalloprotease (MMP) enzyme MMP12 at a ratio of 1% total MMP to protein construct (wt/wt) in Tris buffered saline (pH7.4) containing 5 mM CaCl₂). The reactions were stopped by the addition of 20 mM EDTA and then samples tested for binding or functional activity as described.

### T-Cell Activation Bioassay

Functional activity of protein constructs was assessed in a co-culture assay using MDA-MB-231 (PD-L1 high) or A549 cells (PD-L1 low) with an NFAT-RE-luciferase Jurkat reporter cell line (Promega—TCR/CD3 Effector cells NFAT). MDA-MB-231 (PD-L1 high) or A549 cells (40000 cell/well) were seeded into 96-well white clear bottomed tissue culture treated plates in Hybri-Care medium (ATCC) supplemented with 10% FBS and incubated at 37° C. overnight in a CO2 incubator. Medium was removed and control antibodies or protein constructs (+/−pre-digestion with MMP3/7/12) prepared in assay medium (RPMI supplemented with 10% FBS) were added to the cells. TCR/CD3 Effector cells (NFAT) were thawed and diluted according to the manufacturer's protocol, then added to the assay wells. Following a 6-hour incubation at 37° C. in a CO2 incubator the plates were re-equilibrated to room temperature and luciferase activity determined by addition of Bio-Glo reagent for 5-10 minutes and measurement of luminescent signal (RLU). Fold induction was determined by calculating the ratio of the sample RLU/RLU in the absence of antibody following subtraction of background luminescence signal.

### ELISA Binding of IgG and LB Proteins

For ELISA binding assays, 384 well or 96 well plates were coated with lug/ml and incubated overnight at 4° C., protected from light. Plates were washed twice with PBS/0.05% Tween 20 before blocking with block buffer (3% milk protein in PBS) for 1 hour at room temperature. Next, test agent or controls were loaded and incubated for 1 hour at room temperature. Plates were then washed three times with PBS/0.05% Tween 20. Finally, HRP conjugated secondary antibody, diluted 1/5,000 in block buffer, was added and incubated for 1 hour at room temperature. Plates were again washed three times with PBS/0.05% Tween before addition of TMB substrate and incubation for 6 minutes at room temperature. Subsequently, the reaction was stopped with stop solution. Absorbance was read at 450 nm and 570 nm. Absorbance at 570 nm was subtracted from absorbance at 450 nm for each plate.

### Cell Binding of LB Proteins

Jurkat cells were harvested, counted and washed once with PBS, before staining with Zombie UV viability dye diluted 1 in 1000 in PBS for 30 minutes at room temperature (RT). Induction was determined by calculating the ratio of the sample RLU/RLU in the absence of antibody following subtraction of background luminescence signal. Cells were washed with staining buffer (0.1% BSA in PBS), resuspended in staining buffer and separated into test aliquots. Cells were incubated with LB proteins at the indicated times for 30 min on ice. Cells were washed with staining buffer twice, before incubating with secondary antibody, or staining buffer alone, for 60 minutes on ice. Following secondary antibody incubation, cells were washed twice with staining buffer before fixing with 4% PFA for 10 minutes at RT. Fixed cells were resuspended in staining buffer and stored at 4° C. prior to analysis using the BD LSR Fortessa flow cytometer. For testing PD-L1 expression on A549, MDA-MB-231 and RKO cells, cells were seeded and incubated with 20 ng/ml IFNγ for 45 h before washing once with PBS and detaching with 1 ml TrypLE™ Express Enzyme at 37° C. for 5 min in a standard incubator. Subsequently, cells were blocked and stained for viability using LIVE/DEAD™ Fixable Green (=FITC) and incubation for 20 min in a fridge. Labelled clone MIH2 (to detect PD-L1 expression) or isotype IgG was incubated with viability-stained cells for 30 min at 4° C. Samples were centrifuged and washed twice before measuring on BD FACS Canto II.

### Surface Plasmon Resonance of Fab Proteins

In order to assess the binding of test Fabs to human CD3 (Acro Biosystems, Newark, USA) and cynomolgus CD3 (Acro Biosystems, Newark, USA), multi-cycle kinetic analysis was performed at 25° C. on a Biacore® 8K (serial no. 2724204) HBS-P+(Cytiva, Marlborough, USA) supplemented with 0.1% BSA (Sigma, Dorset, UK) was used as running buffer as well as for ligand and analyte dilutions. The Fab samples were diluted to 1.0 μg/mL in running buffer and at the start of each cycle loaded onto F_{c}2, of a series S CM5 chip (Cytiva, Marlborough, USA) previously coupled with an anti-human Fab capture antibody (Cytiva, Marlborough, USA) using standard amine chemistry. Ligand was captured at a flow rate of 10 μl/min to give an immobilization level (R_{L}) of ˜110 RU. The surface was then allowed to stabilize. Multi-cycle kinetic data was obtained using either human or cynomolgus protein as the analyte injected at a flow rate of 30 μl/min to try and minimize any potential mass transfer effects. An eight point, two-fold dilution range from 100.0 nM to 0.78 nM was prepared in running buffer for antigens. For each concentration, the association phases were monitored for 240 seconds, and the dissociation phase was measured for 600 seconds. Regeneration of the sensor chip surface was conducted between cycles using 10 mM glycine pH 2.1. Multiple repeats of a blank and of antigen were programmed into the kinetic run, in order to check the stability of both the surface and analyte over the kinetic cycles. The signal from the reference F_{c}1 (no ligand captured) was subtracted from that of FE2 to correct for bulk effect and differences in non-specific binding to a reference surface. The signal from each blank run (ligand captured but no antigen) was subtracted to correct for differences in surface stability. Binding was analysed using 1:1 binding analysis due to the high affinity interaction between the antibody and antigen. The data was analysed using a procedure referred to as double referencing. This refers to the process of first subtracting the reference channel response (F_{c}1) and then subtracting the zero concentration sensorgram to compensate for bulk effects, baseline drift and small differences between the reference and the active channel.

### Primary T Cell Killing on A549, MDA-MB-231, RKO

Respective tumor cells were counted and seeded into the appropriate 96-well plates and left to adhere for approximately 24 h. PBMC cells were isolated from whole blood by centrifugation followed immediately by T cell isolation. To this end, PBMCs were incubated with a Pan T cell MicroBead cocktail and subsequently applied to a MACS separator column. Flow through cells representing enriched T cells were collected and diluted to desired levels. For the Incucyte® live cell analysis platform, LB proteins or buffer with appropriate concentrations of Annexin V stain were added to adherent tumor target cells followed immediately by T cells and placement of plates into the Incucyte® S3 Live-Cell Analysis System incubator. Scanning intervals were set at every 4 h for 72-96 h. Annexin V data was normalized against Buffer/T-cell/Tumor cell signals before plotting.

### In Vivo Assessment

Humanized mice were generated by myeloablation, transplantation of hCD34+ HSCs into NCG mice and subsequent myeloid cytokines boost. MDA-MB-231 TNBC cells were implanted and grown to a mean tumor volume of 80 mm³. Mice were then randomized and treated with either IgG isotype control antibody, atezolizumab or LB proteins at indicated doses by intraperitoneal injection, every 3 days, up to eight times. Tumor size was measured every 3 or 4 days using a caliper and mean values were plotted using Graphpad Prism.

### Results and Discussion

### In Silico Affinity Maturation of Anti-PD-L1 Binding to Human and Cynomolgus PD-L1

Sequences of human and cynomolgus (cyno) PD-L1 proteins are provided in Table 10.

TABLE 10
Illustrative PD-L1 sequences

| | | SEQ |
|---|---|---|
| Protein | Sequence | ID NO |
| Human PD- | FTVTVPKDLYVVEYGSNMTIE | 35 |
| L1 amino | CKFPVEKQLDLAALIVYWEME | |
| acids (AA) | DKNIIQFVHGEEDLKVQHSSY | |
| 19-127 | RQRARLLKDQLSLGNAALQIT | |
| | DVKLQDAGVYRCMISYGGADY | |
| | KRIT | |
| Cyno PD-L1 | FTVTVPKDLYVVEYGSNMTIE | 36 |
| | CKFPVEKQLDLTSLIVYWEME | |
| | DKNIIQFVHGEEDLKVQHSNY | |
| | RQRAQLLKDQLSLGNAALRIT | |
| | DVKLQDAGVYRCMISYGGADY | |
| | KRIT | |

Examination of a crystal structure (PDB: 5GGT) of human PD-L1 in complex with the Fab domain of an anti-PD-L1 antibody disclosed in U.S. Pat. No. 7,943,743 B2 (BMS-936559), and a model of the cyno PD-L1 equivalent indicated that the only difference between the human and cyno PD-L1 epitope is Ala51Thr Ala52Ser as indicated in the alignment Table 11.

TABLE 11
Alignment of AA19-127 of full lengths
huPD-L1 and cynomolgus PD-L1 proteins

| | Alignment | |
|---|---|---|
| huPD-L1_19-127 | FTVTVPKDLYVVEYGSNMTIECKFPVEKQLDLAALIVYWEMEDKNIIQFVHGEEDLKVQH | 60 |
| cynoPD-L1 | FTVTVPKDLYVVEYGSNMTIECKFPVEKQLDLTSLIVYWEMEDKNIIQFVHGEEDLKVQH | 60 |
| | ********************************::************************** | |
| huPD-L1_19-127 | SSYRQRARLLKDQLSLGNAALQITDVKLQDAGVYRCMISYGGADYKRIT | 109 |
| cynoPD-L1 | SNYRQRAQLLKDQLSLGNAALRITDVKLQDAGVYRCMISYGGADYKRIT | 109 |
| | *.*****:*************:*************************** | |

huPD-L1_19-127 = SEQ ID NO: 35
cynoPD-L1 = SEQ ID NO: 36

Furthermore, Ala51Thr and Ala52Ser predominantly interact with VH backbone, not amino acid side chains. Together, these facts made differentially influencing BMS-936559 binding to human and cyno PD-L1 difficult. As a result, in silico saturation mutagenesis was performed using Rosetta and MOE software on VH residues proximal to the Ala51Thr Ala52Ser site or elsewhere on the VH paratope to attempt to equalize the binding affinity of BMS-936559 to human and cyno PD-L1. Mutations were selected if a negative (favorable) ddG affinity to cyno PD-L1 was calculated for a variant and the ddG affinity was unchanged or changed only slightly improved for the same variant binding to human PD-L1. A set of 54 variants over 13 residue positions at the paratope of VH of BMS-936559 with PD-L1 were generated, as shown in Table 6. These 54 variants were produced as Fab proteins by transient transfection of CHO cells, then purified using CH1 affinity purification. After purification, uniformity was assessed by analytical SEC (Table 12) and proteins with purities >87% were further evaluated for binding to human and cyno PD-L1 in SPR (Table 13).

TABLE 12
Productivity analyses for Anti-PD-L1 Fab variants

| Name | Main Product (%) | Yield mg/L |
|---|---|---|
| WT | 91.002% | 39.50 mg/L |
| mut-01 | 63.529% | 27.25 mg/L |
| mut-02 | 63.192% | 48.50 mg/L |
| mut-03 | 94.074% | 28.50 mg/L |
| mut-04 | 96.531% | 30.00 mg/L |
| mut-05 | 60.404% | 33.75 mg/L |
| mut-06 | 90.969% | 34.50 mg/L |
| mut-07 | 87.466% | 36.50 mg/L |
| mut-08 | 97.710% | 24.50 mg/L |
| mut-09 | 72.669% | 22.75 mg/L |
| mut-10 | 85.437% | 13.75 mg/L |
| mut-11 | 80.163% | 19.75 mg/L |
| mut-12 | 62.940% | 14.75 mg/L |
| mut-13 | 80.894% | 15.50 mg/L |
| mut-14 | 98.401% | 13.75 mg/L |
| mut-15 | 90.970% | 11.50 mg/L |
| mut-16 | 41.225% | 13.75 mg/L |
| mut-17 | 85.344% | 18.50 mg/L |
| mut-18 | 83.916% | 15.50 mg/L |
| mut-19 | 83.614% | 14.00 mg/L |
| mut-20 | 87.632% | 15.75 mg/L |
| mut-21 | 64.096% | 15.00 mg/L |
| mut-22 | 93.320% | 10.00 mg/L |
| mut-23 | 89.315% | 7.75 mg/L |
| mut-24 | 70.149% | 28.00 mg/L |
| mut-25 | 84.212% | 103.25 mg/L |
| mut-26 | 86.124% | 49.75 mg/L |
| mut-27 | 92.157% | 75.75 mg/L |
| mut-28 | 89.031% | 76.00 mg/L |
| mut-29 | 70.083% | 51.75 mg/L |
| mut-30 | 78.668% | 65.50 mg/L |
| mut-31 | 87.759% | 101.00 mg/L |
| mut-32 | 82.247% | 43.00 mg/L |
| mut-33 | 81.936% | 58.75 mg/L |
| mut-34 | 94.595% | 72.25 mg/L |
| mut-35 | 82.909% | 86.75 mg/L |
| mut-36 | 89.578% | 93.50 mg/L |
| mut-37 | 64.804% | 42.50 mg/L |
| mut-38 | 80.872% | 50.00 mg/L |
| mut-39 | 87.589% | 25.25 mg/L |
| mut-40 | 77.681% | 35.25 mg/L |
| mut-41 | 81.078% | 23.50 mg/L |
| mut-42 | 93.201% | 51.25 mg/L |
| mut-43 | 89.258% | 45.00 mg/L |
| mut-44 | 96.246% | 91.50 mg/L |
| mut-45 | 86.469% | 42.50 mg/L |
| mut-46 | 96.871% | 28.75 mg/L |
| mut-47 | 91.879% | 70.25 mg/L |
| mut-48 | 96.082% | 58.00 mg/L |
| mut-49 | 98.815% | 25.25 mg/L |
| mut-50 | 92.847% | 34.50 mg/L |
| mut-51 | 96.9% | 167.5 mg/L |
| mut-52 | 89.5% | 65.3 mg/L |
| mut-53 | 94.0% | 132 mg/L |
| mut-54 | 96.99% | 168 mg/L |

Table 13 depicts SPR binding result of selected anti-PD-L variants to human and cyno PD-L1 protein. WT affinities were determined as KD (M) 4.4E-10 for human PD-L1 while cyno PD-L1 affinity is approximately 13 fold lower at KD (M) 7.9E-9. Mut-6, -23, -43 showed increased affinities to human and cyno PD-L1, while Mut-50 showed increased affinity to human PD-L1 only. Mut-48 and Mut-49 were affinity improved to cyno, but not human, PD-L1 relative to WT.

TABLE 13
Anti-PD-L1 Fab WT or variants binding to human or
cyno PD-L1 and KD determination in SPR

| Name | Human PD-L1 KD (M) | Cyno PD-L1 KD (M) |
|---|---|---|
| WT | 4.40E−10 | 7.86E−09 |
| mut-3 | 1.10E−09 | 1.36E−08 |
| mut-4 | 8.57E−10 | 1.08E−08 |
| mut-6 | 8.57E−11 | 2.86E−09 |
| mut-7 | 4.25E−09 | 1.43E−07 |
| mut-8 | 4.89E−10 | 6.48E−09 |
| mut-14 | 3.36E−09 | 2.93E−08 |
| mut-15 | 1.69E−09 | 1.05E−08 |
| mut-20 | 8.75E−10 | 1.59E−08 |
| mut-22 | 1.84E−09 | 2.00E−08 |
| mut-23 | 1.95E−10 | 3.02E−09 |
| mut-27 | 5.58E−09 | ND |
| mut-28 | 5.01E−10 | 5.51E−09 |
| mut-31 | ND | ND |
| mut-34 | 3.58E−09 | 5.82E−09 |
| mut-36 | ND | ND |
| mut-39 | 1.54E−09 | 3.96E−09 |
| mut-42 | 3.99E−09 | 5.98E−09 |
| mut-43 | 2.04E−10 | 2.73E−09 |
| mut-44 | 5.42E−10 | 6.85E−09 |
| mut-46 | 1.04E−09 | 1.27E−08 |
| mut-47 | 5.94E−10 | 9.32E−09 |
| mut-48 | 2.49E−10 | 3.24E−09 |
| mut-49 | 2.24E−10 | 3.53E−09 |
| mut-50 | 1.24E−10 | 4.82E−09 |
| mut-51 | 1.57E−11 | 1.8E−10 |
| mut-52 | 2.14E−11 | 2.98E−10 |
| mut-53 | 2.17E−11 | 2.88E−10 |
| mut-54 | 7.22E−11 | 6.31E−10 |

ND = binding not detected

### LB Protein Construct Design Cloning, Expression, and Characterization

To produce proteins for functional testing, DNA cassettes for each construct type were designed using combinations of the anti-PD-L1 variable domain, constant domain and linker sequences found in Table 1, in combination with the murine or humanized variable domain variants of the anti-CD3 antibody SP34, as shown in Table 2. To ensure the fitness of the SP34-derived domains for the structural format of these proteins, a series of novel SP34 humanization variants were tested alongside known SP34 V domain sequences for the murine and humanized forms as controls (Table 2). These humanized variants were then combined with PD-L1 variable domains, constant domains and linkers to form full-length heavy and light chain sequences (Table 3). Using these full-length chains, 16 initial designs were synthesized and cloned into expression vectors encoding human IgG1-based heavy and light chain sequences and a free hinge-Fc fragment (Table 4). The anti-PD-L1 variable domain sequences used in the protein construct disclosed herein are the variable domain sequences provided in U.S. Pat. No. 7,943,743 B2 and Table 5 and Table 6. The proteins were produced by transient transfection of CHO cells, then purified by proA, IEX and/or SEC. After the ProA step, proteins were examined for yield and uniformity by SEC (Table 14). Fully purified proteins demonstrated high purity (>95%) and uniformity by analytical SEC, demonstrating that the best-behaved constructs can be expressed and purified as an intact, stable product, in a single process.

TABLE 14
Productivity analyses for selected LB proteins, sequence
combinations as shown in Table 9

| | | Main Product | | |
|---|---|---|---|---|
| Name | Titer (mg/L) | (%) | HMW (%) | LMW (%) |
| LB201 | 250 | 42.8 | 53 | 4.2 |
| LB202 | 280 | 32 | 62.8 | 5.2 |
| LB203 | 260 | 36 | 62 | 2 |
| LB204 | 180 | 59.6 | 34.5 | 5.9 |
| LB205 | 270 | 38.3 | 59.4 | 2.3 |
| LB206 | 180 | 80.5 | 18.5 | 1 |
| LB207 | 240 | 44.5 | 54.5 | 1 |
| LB208 | 260 | 49.2 | 50.8 | 0 |
| LB209 | 290 | 43 | 51.4 | 5.6 |
| LB210 | 220 | 46.5 | 53.2 | 6.3 |
| LB211 | 270 | 41 | 54.3 | 4.7 |
| LB212 | 240 | 43.2 | 53.8 | 3 |
| LB213 | 260 | 45.2 | 50.5 | 4.3 |
| LB214 | 180 | 32.5 | 66.3 | 1.2 |
| LB215 | 210 | 45.1 | 49.2 | 5.7 |
| LB216 | 181 | NA | NA | NA |

HMW = Higher Molecular Weight Product
LMW = Lower Molecular Weight Product

### In Vitro Functional Characterization

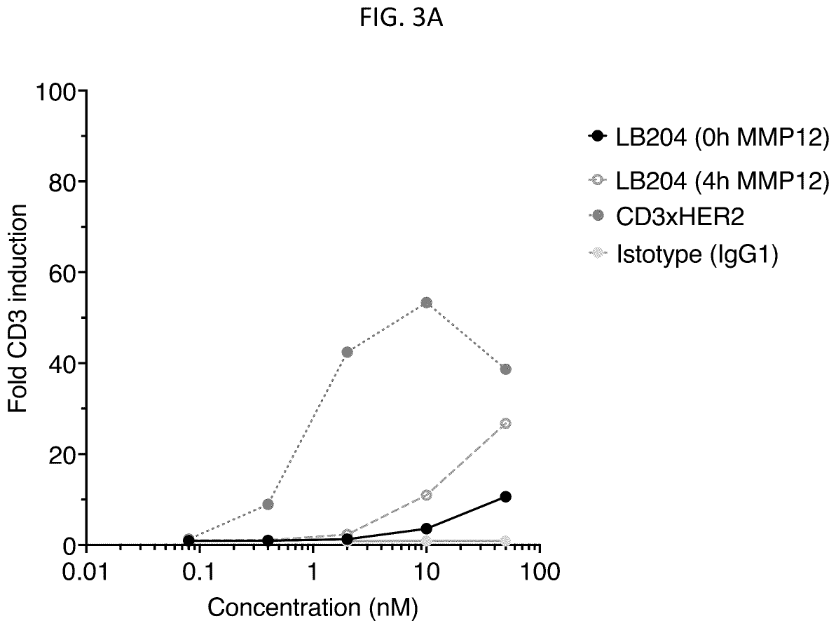

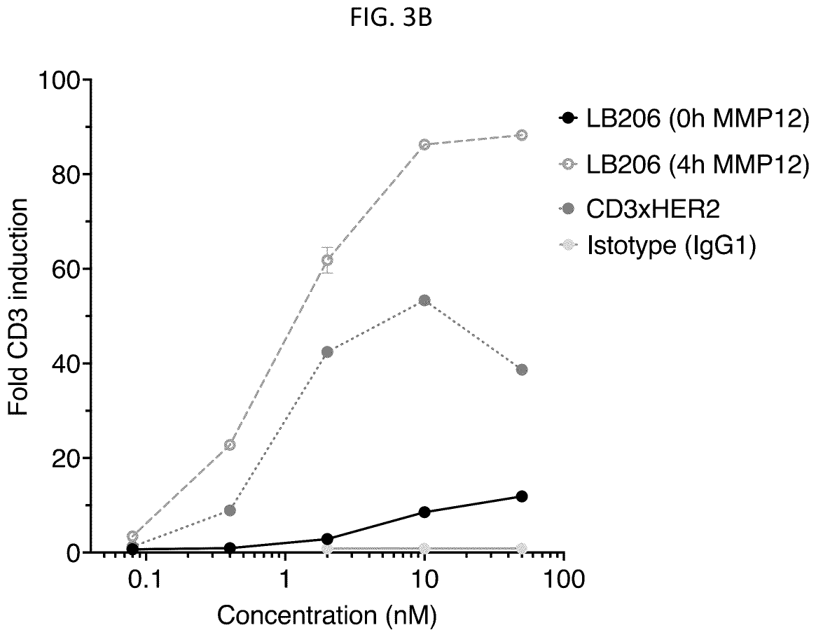

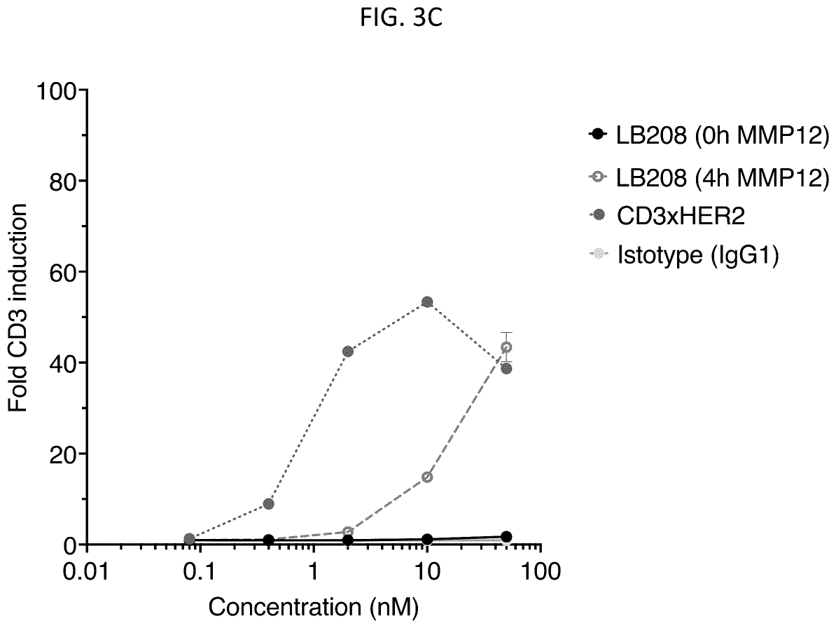

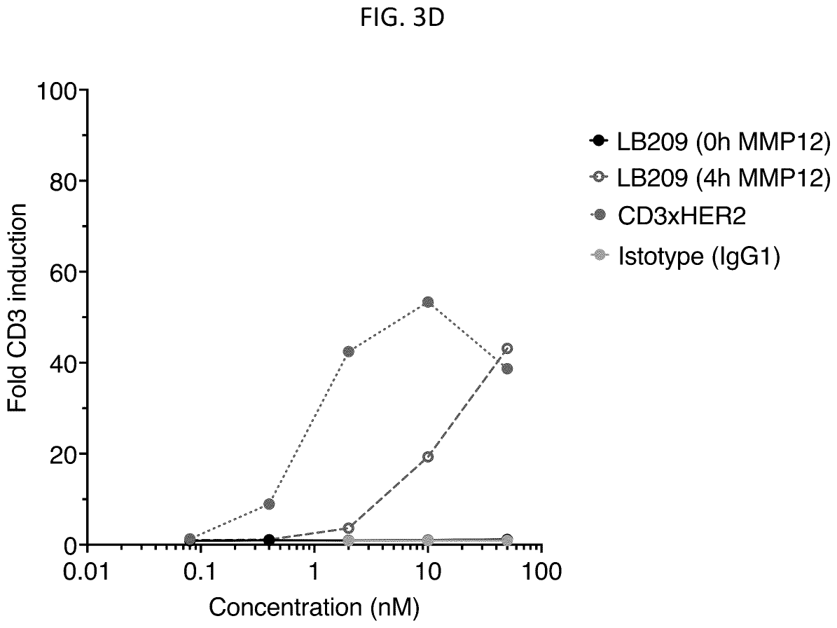

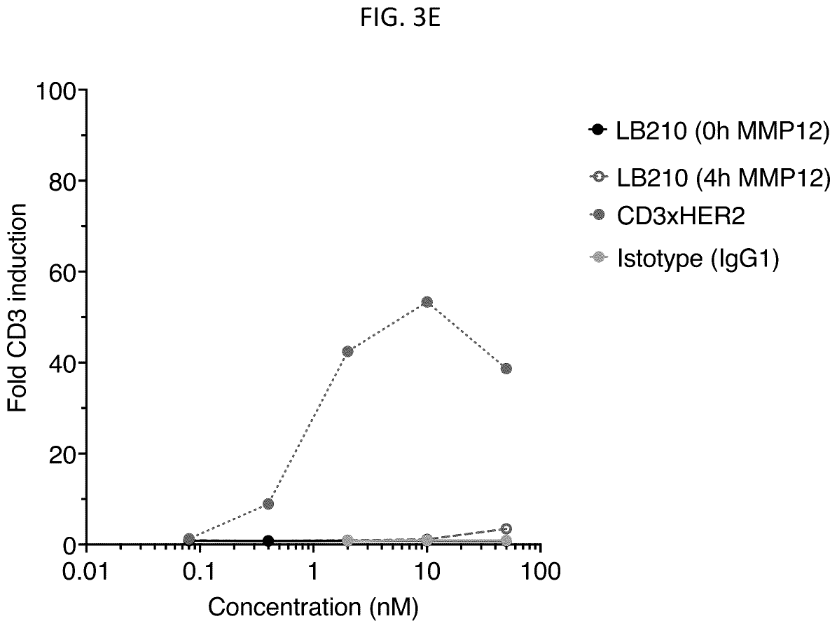

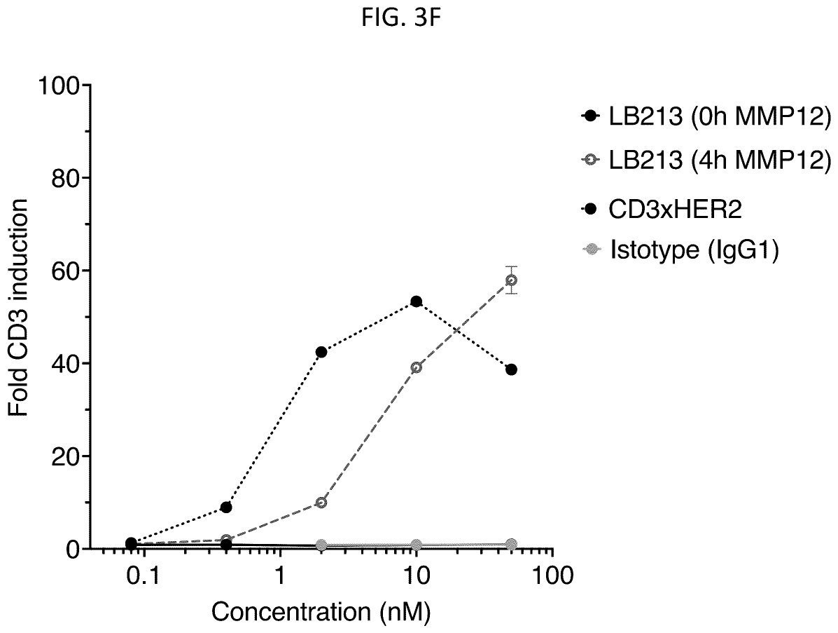

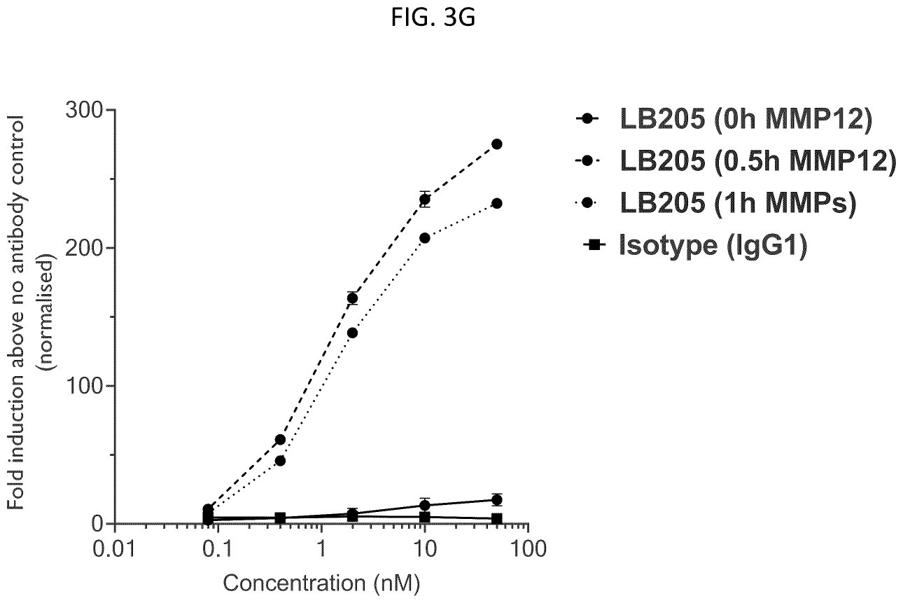

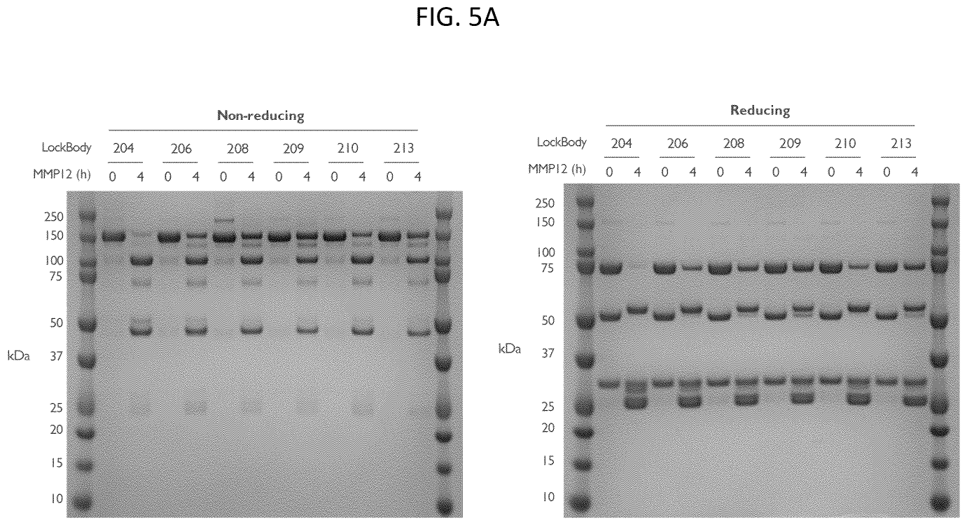

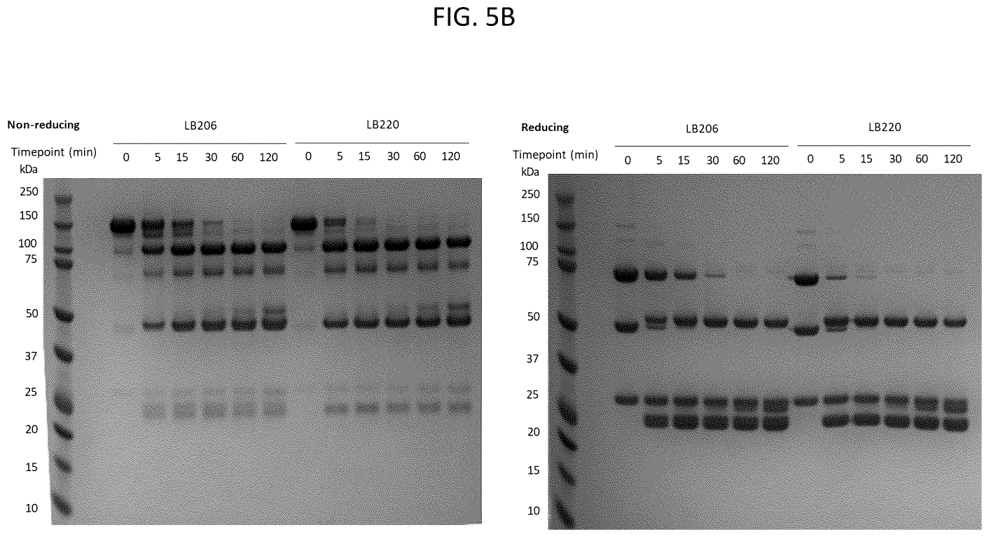

Purified proteins with higher yield and/or uniformity characteristics in Table 13 (LB204, LB205, LB206, LB208, LB209, LB210 and, LB213) were incubated at 37° C. for 0 or 4 h in the presence or absence of human MMP12 enzyme. FIG. 5A shows digestion profiles of exemplar LB proteins (LB204, 206, 208, 209, 210, 213 and 220). Intact proteins showed the expected band patterns in reducing and non-reducing SDS PAGE with one predominant band at 150 kDa visible in non-reducing, and 3 chains visible at 75 kDa (LB heavy chain), 50 kDa (LB light chain) and ˜30 kDa (Fc Stump) in reducing gels. Upon incubation with MMP12 at 37° C., marked differences in band distribution compared to intact proteins suggested cleavage of both LB heavy chains and LB light chains. In reducing SDS-PAGE, the appearance of multiple 25-30 kDa bands (corresponding to cleaved V-C domain from either LB light or heavy chain, and proteolytically-released single chain Fc fragments) and ˜55 kDa band (corresponding to VH-CH domain with Fc), and corresponding disappearance of intact LB light and heavy chain products at 75 and 50 kDa, demonstrates cleavage of either intact chain in both of the linkers, thereby creating the respective products as depicted in FIG. 1A. Selected LB proteins were then tested for CD3 activation signal in mixed cell culture assay using the human PD-L1+ cell line MDA-MB-231 and a Jurkat cell line engineered to provide reporter signal for human CD3 activation (FIG. 3A-FIG. 3G). This assay therefore tests the ability of molecules to bind the cell surface of the MDA-MB-231 cell line (through PD-L1 for test articles or Her2 for the BiTE control protein), and to activate CD3 in the Jurkat reporter line through trans presentation of the CD3 binding domains. The Her2/CD3 BiTE positive control protein induced strong, concentration dependent CD3 activation, while the IgG1 isotype negative control did not induce any signal. All test article samples exhibited low or no CD3 activation signal at 0 h MMP12 incubation (i.e., intact, uncleaved protein), demonstrating that the SP34 domains which bind CD3 have minimized ability to bind CD3 in all constructs. Unexpectedly, however, only proteins LB206 and LB213 exhibited the desired combination of low/no CD3 signal at 0 h, with high, concentration-dependent activation at 4 h. Indeed, construct LB210 demonstrated no signal after activation, at all (FIG. 3A-FIG. 3G). These findings demonstrated that both linker type and humanization sequence for the CD3 variable domains are critical factors in performance of the resulting molecules.

The anti CD3 SP34 humanization V domain variants contained in LB206 and LB213 were then characterized further in Fab and IgG formats. First, surface plasmon resonance (SPR) of purified Fab proteins was used to confirm binding to commercially available human and cyno CD3δε heterodimer peptides (FIG. 4A and B). While K_{D} values were calculated as 29 nM and 46 nM respectively, the off rate of Anti-CD3-Fab-001 was notably different from Anti-CD3-Fab-002 (4.7×10⁻³ vs 2.0×10⁻²) against human CD3δε heterodimer. In contrast, purified IgGs of CD3-IgG00-1 and CD3-IgG-002 showed no difference in apparent affinities in a binding ELISA to the same commercially available human and cyno CD3δε heterodimer proteins (FIG. 4C). Finally, purified IgG proteins were tested for CD3 activation signal in a Jurkat cell line engineered to provide reporter signal for human CD3 activation (FIG. 4D). This assay tested the ability of the new humanization variants to activate CD3 in the Jurkat reporter cell line in direct comparison to the mouse SP34 IgG molecule. These findings showed that Anti-CD3-IgG-001 displays a similar activation ability to SP34 (EC50 of either molecule in this assay was 0.2 nM) in the Jurkat reporter cell line, while Anti-CD3-IgG-002 showed an approximately 5-fold lower apparent EC50 in this assay (EC50 0.9 nM). Taken together, these results demonstrated that CD3 dependent agonistic property of SP34 is preserved in Anti-CD3-IgG-001 humanization, while anti-CD3-IgG-002 showed a 5-fold lower agonistic potential in Jurkat reporter cells, in keeping with the lower K_{D} and faster K_{d} rate observed in SPR analysis.

Having identified LB206 and LB213 as functional molecules with desired activities, additional proteins were designed on the basis of these for expression and subsequent functional testing. As before, DNA cassettes for each construct type and different modality (FIG. 1B) were designed using combinations of the variable domains, constant domains and linker sequences found in Table 1. The proteins were produced by transient transfection of CHO cells, then purified by ProA, IEX and/or SEC. After the ProA step, proteins were examined for yield and uniformity by SEC (Table 15). Significant differences in yields were observed for some modalities over others but all fully purified proteins demonstrated high purity (>95%) and uniformity by analytical SEC, demonstrating that the best-behaved constructs can be expressed and purified as an intact, stable product, in a single process.

TABLE 15
Productivity analyses for additional selected LB proteins

| | Titer | Main Product | | |
|---|---|---|---|---|
| Name | (mg/L) | (%) | HMW (%) | LMW (%) |
| LB217 | 595 | 21.3 | ND | ND |
| LB218 | 757 | 38.1 | ND | ND |
| LB219 | 329 | 25.8 | ND | ND |
| LB220 | 348 | 30.6 | ND | ND |
| LB221 | 552 | ND | ND | ND |
| LB222 | 624 | ND | ND | ND |
| LB223 | 530 | ND | ND | ND |
| LB224 | 399 | ND | ND | ND |
| LB225 | 635 | ND | ND | ND |
| LB226 | 484 | ND | ND | ND |
| LB227 | 710 | ND | ND | ND |
| LB228 | 685 | ND | ND | ND |
| LB229 | 608 | ND | ND | ND |
| LB230 | 705 | ND | ND | ND |
| LB231 | 759 | ND | ND | ND |
| LB232 | 128 | 37.45 | 49.42 | 13.13 |
| LB233 | 62 | 41.41 | 43.17 | 15.42 |
| LB234 | 288 | 32.67 | 56.39 | 10.39 |
| LB235 | 170 | 35.76 | 49.07 | 15.17 |
| LB236 | 183 | 33.90 | 52.61 | 13.49 |
| LB237 | 456 | 36.66 | 52 | 11.43 |
| LB238 | 218 | ND | ND | ND |
| LB239 | 387 | 36.52 | 50.58 | 12.99 |
| LB240 | 2540 | 21.1 | 34.1 | 44.8 |
| LB241 | 940 | 33.7 | 50.69 | 15.61 |
| LB242 | 931 | 23.1 | 57.41 | 19.49 |
| LB243 | 490 | 22.01 | 53.34 | 24.65 |
| LB244 | 329 | 31.3 | 60.8 | 7.9 |

HMW = Higher Molecular Weight Product
LMW = Lower Molecular Weight Product
ND = No data

Selected molecules were again incubated at 37° C. for the indicated times in the presence or absence of human MMP12 enzyme and digestions profiles were assessed using SDS-PAGE under reducing and non-reducing conditions (FIGS. 5A and 5B). FIG. 5A shows digestion profiles of exemplar LB proteins (LB204, 206, 208, 209, 210, 213, 220). Intact proteins showed the expected band patterns in reducing and non-reducing SDS PAGE with one predominant band at 150 kDa visible in non-reducing, and 3 chains visible at 75 kDa (LB heavy chain), 50 kDa (LB light chain) and ˜30 kDa (Fc Stump) in reducing gels (FIG. 5A). Upon incubation with MMP12 at 37° C., marked differences in band distribution compared to intact proteins suggested cleavage of both LB heavy chains and LB light chains. In reducing SDS-PAGE, the appearance of multiple 25-30 kDa bands (corresponding to cleaved V-C domain from either LB light or heavy chain, and proteolytically-released single chain Fc fragments) and ˜55 kDa band (corresponding to VH-CH domain with Fc), and corresponding disappearance of intact LB light and heavy chain products at 75 and 50 kDa, demonstrates cleavage of either intact chain in both of the co linkers, thereby creating the respective products as depicted in FIG. 1A. Further enzyme digests and SDS-PAGE analyses (FIG. 5B) demonstrated the differential speeds at which different linker designs are cleaved by MMP12. For example, comparison of LB206 and LB220 in this manner showed a rapid disappearance of intact heavy chain of LB220 within 15 min while significant amounts were retained in LB206 digests. This suggests that LHL linkers present in LB220 are more susceptible to MMP12 cleavage than LHL linkers present in LB206.

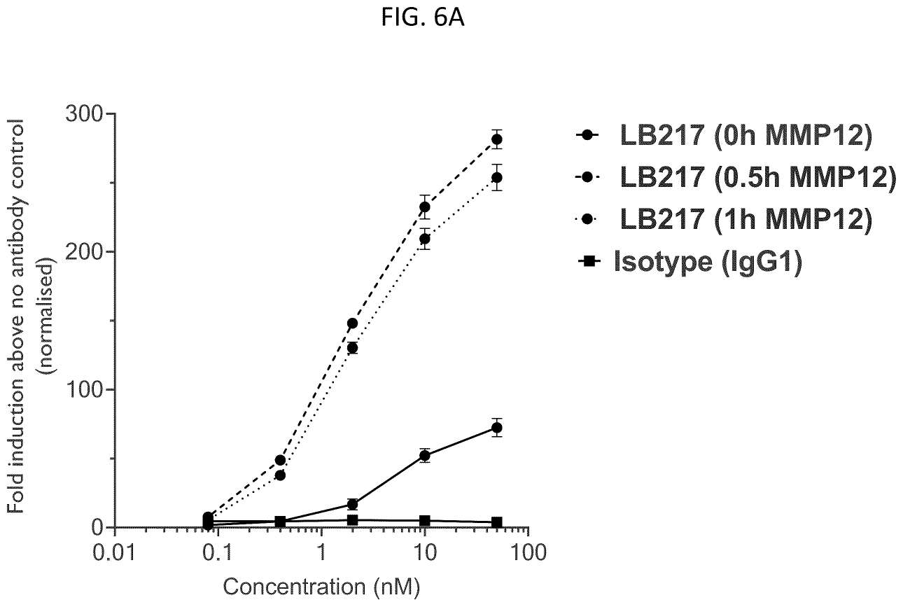

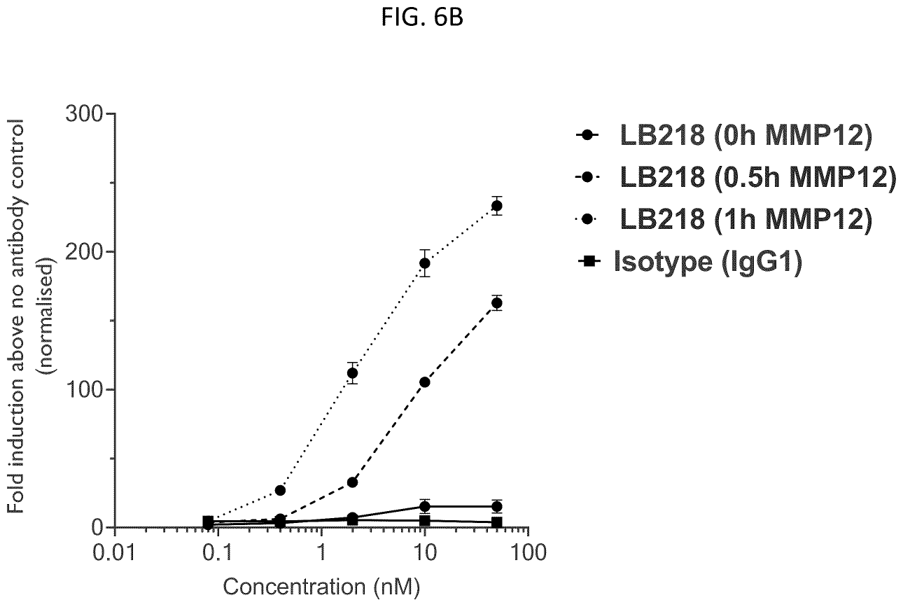

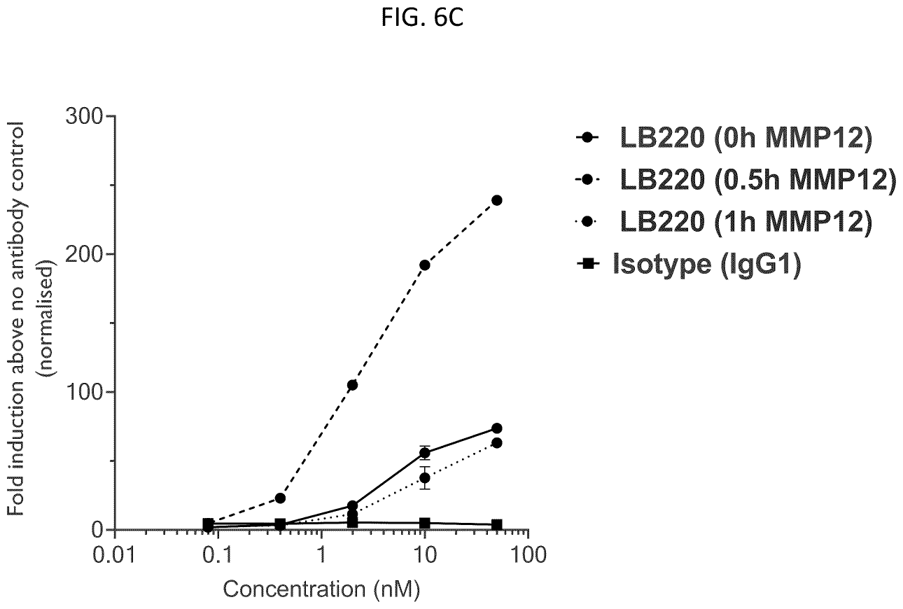

Further, some additional exemplary LB proteins with differing LHL linker and CD3 V domain combinations were selected based on protein expression yields, final purity, and uniformity. Digestion profiles performed in parallel were compared in a screening assay for functionality. To this end, LB217, LB218 and LB220 were tested for CD3 activation signal in mixed cell culture assay using the human PD-L1+ cell line MDA-MB-231 and a Jurkat cell line engineered to provide reporter signal for human CD3 activation (FIG. 6A-FIG. 6C). IgG1 isotype negative control did not induce any signal while all test items exhibited low CD3 activation signal at 0 h MMP12 incubation time (i.e., intact, uncleaved protein), demonstrating that the SP34 domains which bind CD3 have minimized ability to bind CD3 in all constructs. In keeping with the previously observed digestion profile of LB220, luciferase signal in Jurkat reporter cells disappeared rapidly with increasing MMP12 incubation time (0.5 vs 1 h) (FIG. 6C) compared to LB217 (FIG. 6A) and LB218 (FIG. 6B). In addition, LB217 containing humanized CD3 variable domains based on LB206 showed the highest maximal signal compared to LB213 based CD3 variable domain containing proteins LB218 and LB220. Taken together, these findings demonstrated and confirmed that both linker type and humanization sequence for the CD3 variable domains are critical factors in the performance and activation profile of resulting LB molecules.

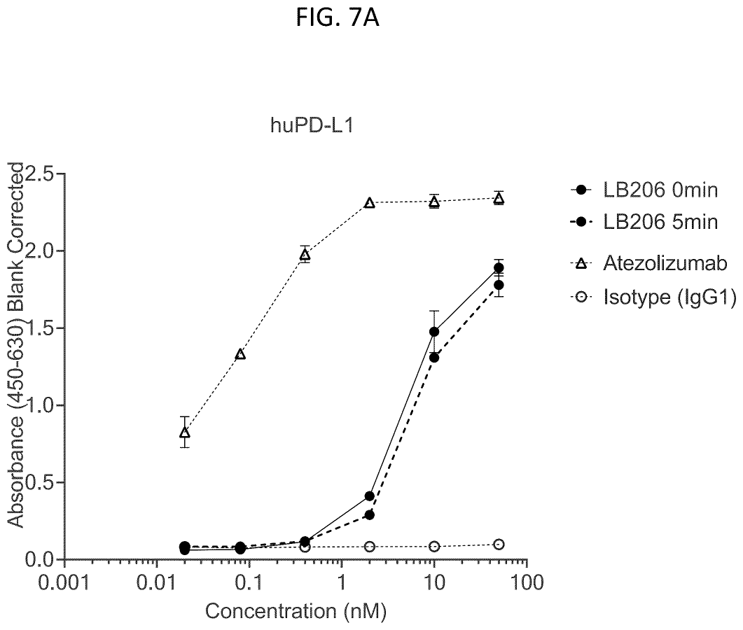

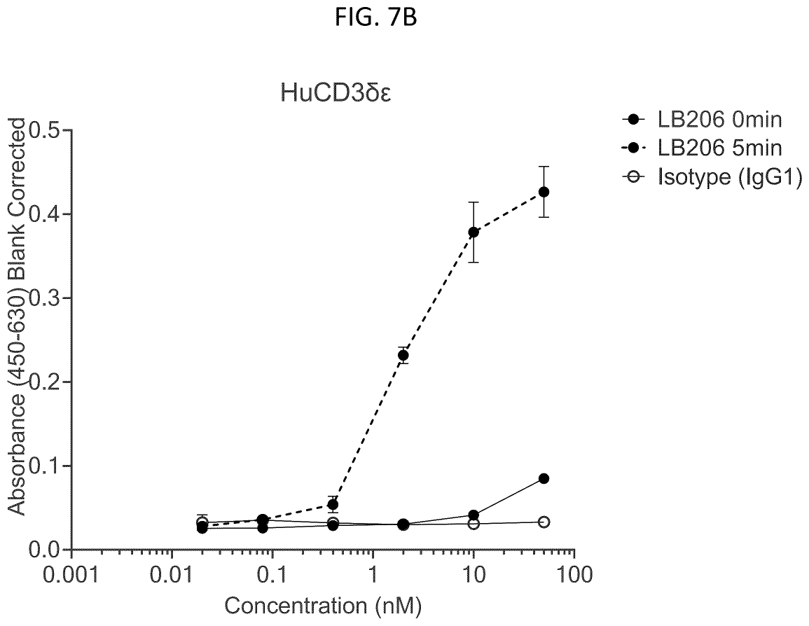

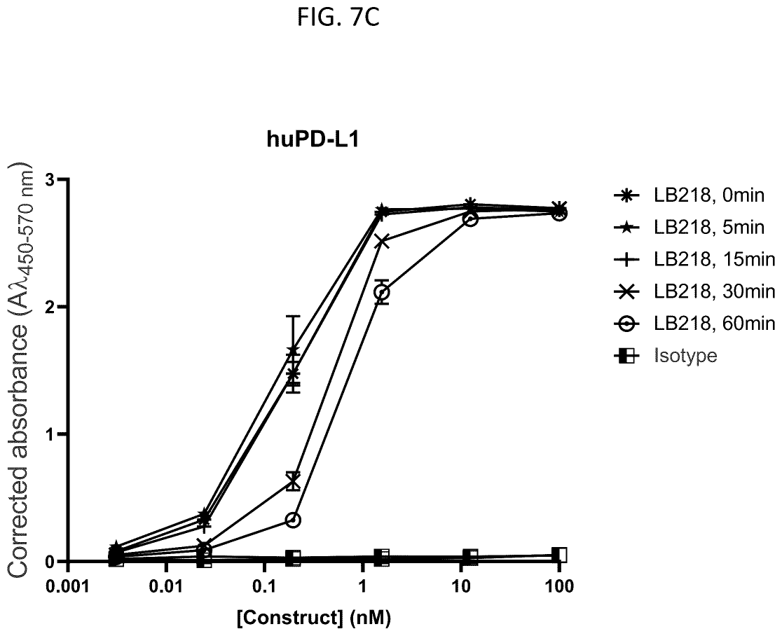

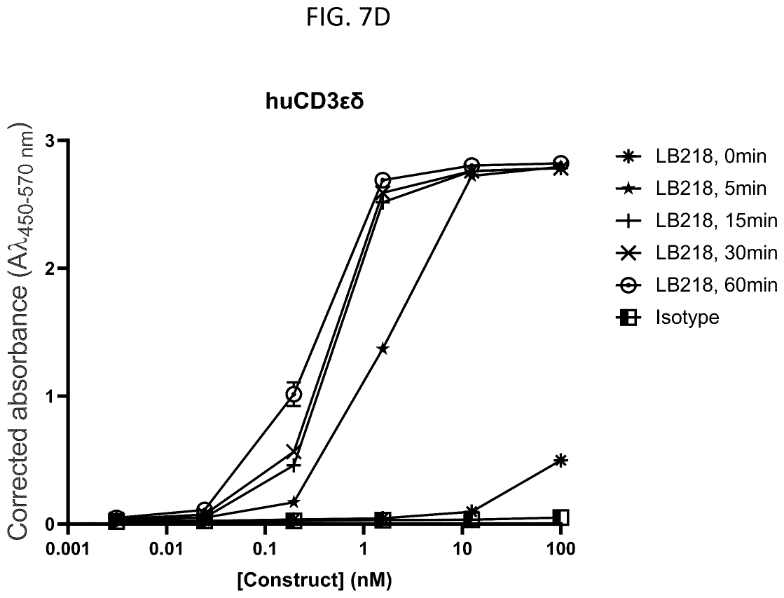

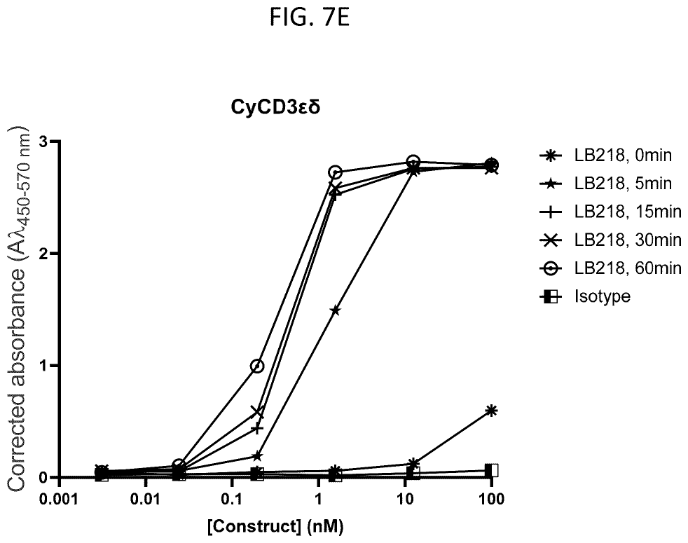

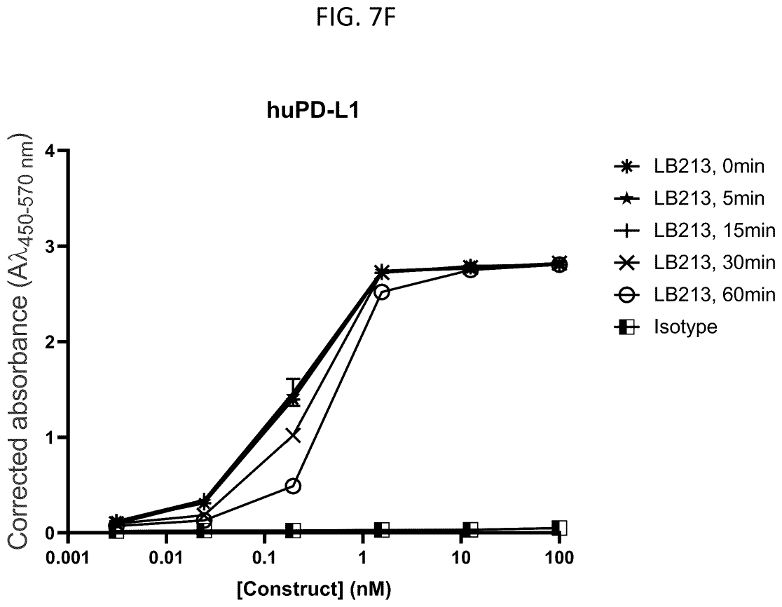

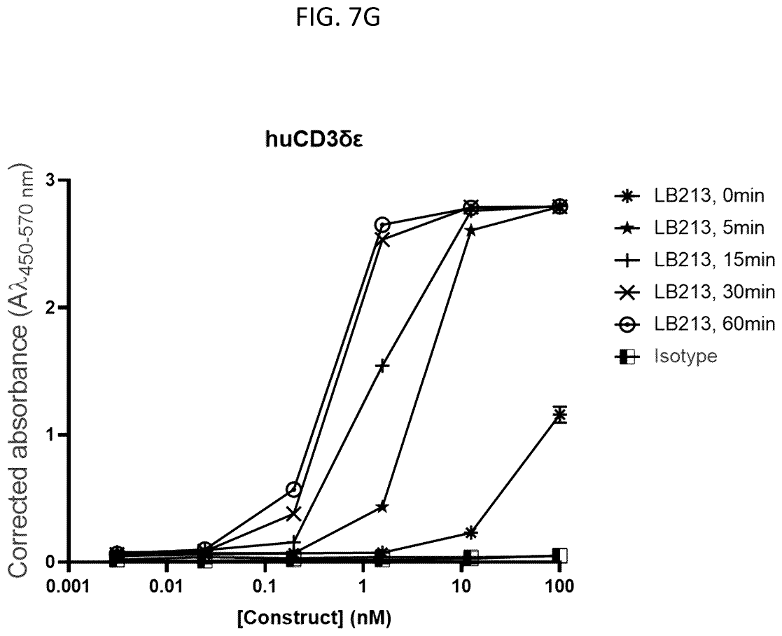

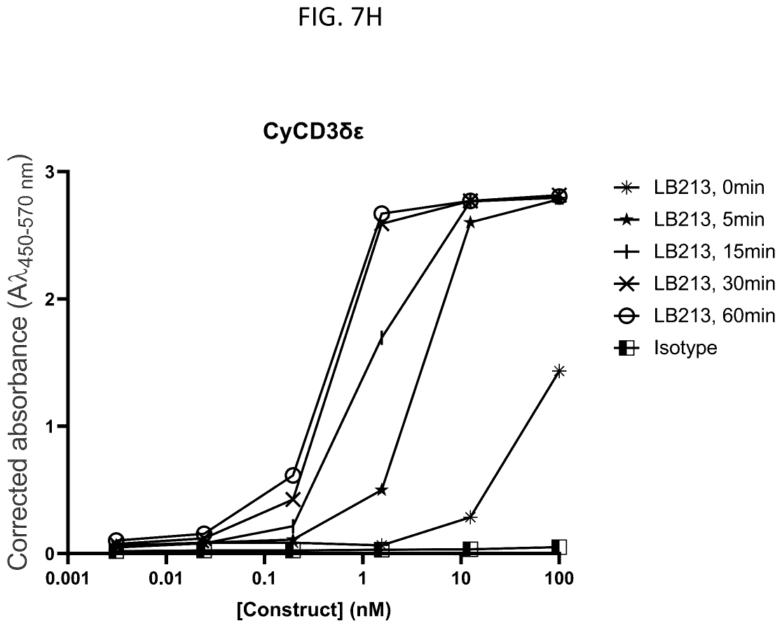

To further assess the ability of undigested fully intact and MMP12 incubated proteins to interact with CD3 and PD-L1, binding ELISAs to recombinant human and cyno CD3δε heterodimers as well as huPD-L1 domain were conducted with exemplary proteins (LB206, LB213, LB220; FIG. 7). Binding signals on both CD3 orthologues confirmed that CD3 binding is greatly diminished in fully intact proteins but strongly increased after incubation with MMP12 at 37° C. at the indicated times. In contrast, huPD-L1 binding of LB proteins is not affected by incubation with MMP12 for up to 15 min but then reduces overtime. This is in keeping with cleavage patterns observed in SDS PAGE analysis which shows clearly diminished levels of intact heavy and lights chains with increasing digestion times and further confirms that both linkers are proteolytically labile. Specifically, FIG. 7A shows ELISA binding of intact or 5 min MMP12 incubated LB206 protein to human PD-L1 with atezolizumab as positive control or IgG1 isotype as negative control (no signal). Intact and MMP12 treated LB206 bind to PD-L1 to a similar level. Binding to PD-L1 appears lower than atezolizumab, consistent with one-arm LB protein structure and to two-arm IgG atezolizumab. FIG. 7B depicts ELISA binding of intact or 5 min MMP12 incubated LB206 protein to human CD3 δε heterodimer or IgG1 isotype as negative control (no signal). Intact LB206 binds to human CD3 δε heterodimer to very low level. In contrast, MMP12 treated LB206 binds strongly binds human CD3 δε heterodimer. FIG. 7C depicts ELISA binding of intact or 5 min, 15 min, 30 min or 60 min MMP12 incubated LB218 protein to human PD-L1 with IgG1 isotype as negative control (no signal). Intact and 5 or 15 min MMP12 treated LB218 bind to PD-L1 to a similar level. While binding to PD-L1 appears lower for 30 min and 60 min, suggesting occurrence of protein cleavage 2 (FIG. 1A). FIG. 7D depicts ELISA binding of intact or 5 min, 15 min, 30 min or 60 min MMP12 incubated LB218 protein to human CD3 δε heterodimer with IgG1 isotype as negative control (no signal). Intact LB218 shows binding to human CD3 SC heterodimer to very low level. With increasing MMP12 treatment times, higher binding to human CD3 δε heterodimer can be observed. 60 min MMP12 treated LB218 shows highest binding to human CD3 δε heterodimer. FIG. 7E depicts ELISA binding of intact or 5 min, 15 min, 30 min or 60 min MMP12 incubated LB218 protein to cyno CD3 SC heterodimer with IgG1 isotype as negative control (no signal). Intact LB218 shows binding to cyno CD3 δε heterodimer to very low level. With increasing MMP12 treatment times, higher binding to cyno CD3 δε heterodimer can be observed. 60 min MMP12 treated LB218 shows highest binding to cyno CD3 δε heterodimer. FIG. 7F depicts ELISA binding of intact or 5 min, 15 min, 30 min or 60 min MMP12 incubated LB213 protein to human PD-L1 with IgG1 isotype as negative control (no signal). Intact and 5 or 15 min MMP12 treated LB213 bind to PD-L1 to a similar level. While binding to PD-L1 appears lower for 30 min and 60 min, suggesting occurrence of protein cleavage 2 (FIG. 1A). FIG. 7G depicts ELISA binding of intact or 5 min, 15 min, 30 min or 60 min MMP12 incubated LB213 protein to human CD3 δε heterodimer with IgG1 isotype as negative control (no signal). Intact LB213 shows binding to human CD3 SC heterodimer to very low level. With increasing MMP12 treatment times, higher binding to human CD3 δε heterodimer can be observed. 60 min MMP12 treated LB213 shows highest binding to human CD3 δε heterodimer. FIG. 7H depicts ELISA binding of intact or 5 min, 15 min, 30 min or 60 min MMP12 incubated LB213 protein to cyno CD3 Sc heterodimer with IgG1 isotype as negative control (no signal). Intact LB213 shows binding to cyno CD3 δε heterodimer to very low level. With increasing MMP12 treatment times, higher binding to cyno CD3 δε heterodimer can be observed. 60 min MMP12 treated LB213 shows highest binding to cyno CD3 δε heterodimer.

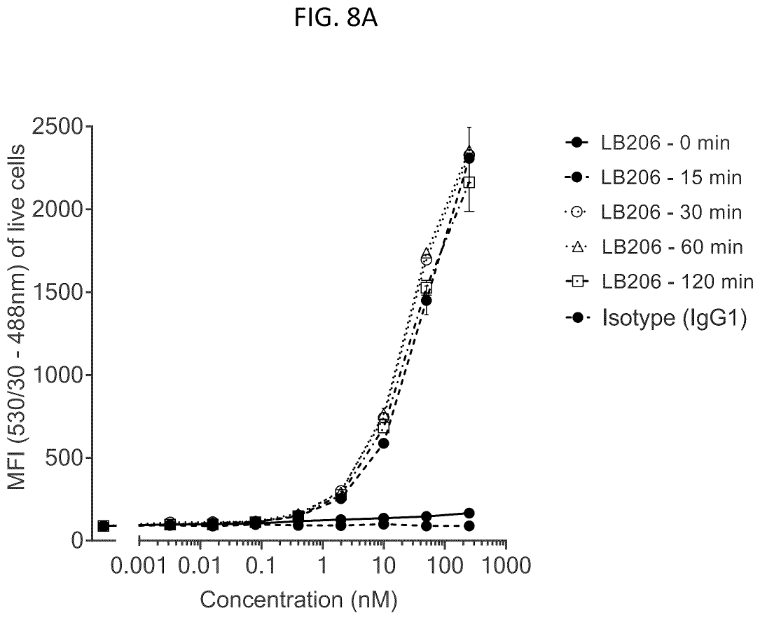

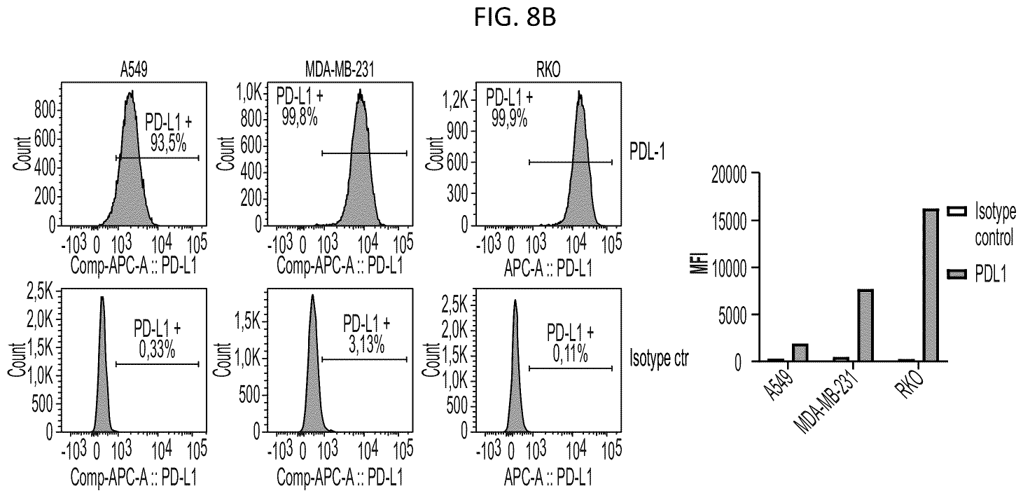

Next, Jurkat cell binding assays were performed for LB206 using flow cytometry and showed a corresponding profile of binding only after MMP12-mediated LB hinge linker digestion (FIG. 8). These results demonstrate that the intact LB206 protein has low or negligible binding capacity on a CD3+/PD-L1− cell. They further confirm that the observed low or no activation of luciferase signal in Jurkat reporter cell line-based activation assays of intact/undigested LB proteins is due to inefficient or absent presentation of CD3 binding in trans.

### Potency of Lead Molecules in Primary T Cell Assays

To further increase understanding of LB protein functionality, selected LB proteins were assessed for their ability to induce tumor cell killing by primary T cells. Three representative cancer cell lines based on their PD-L1 expression levels were selected as target cells.

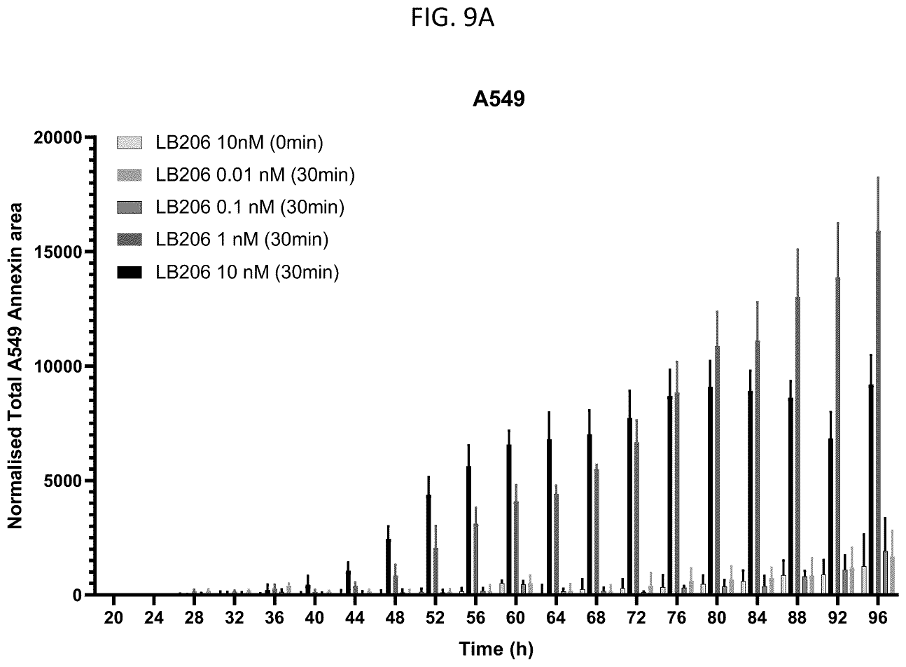

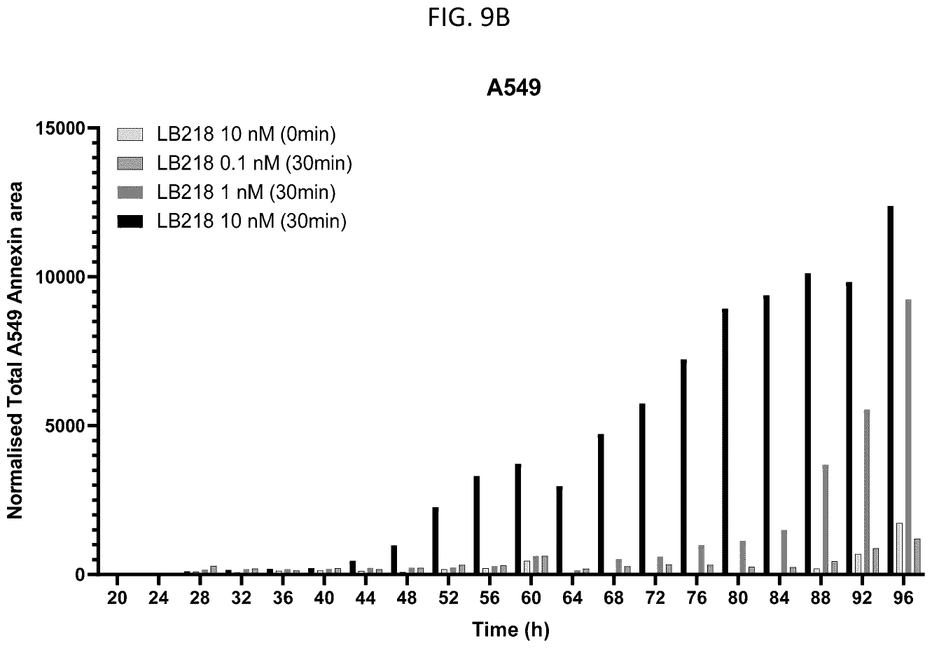

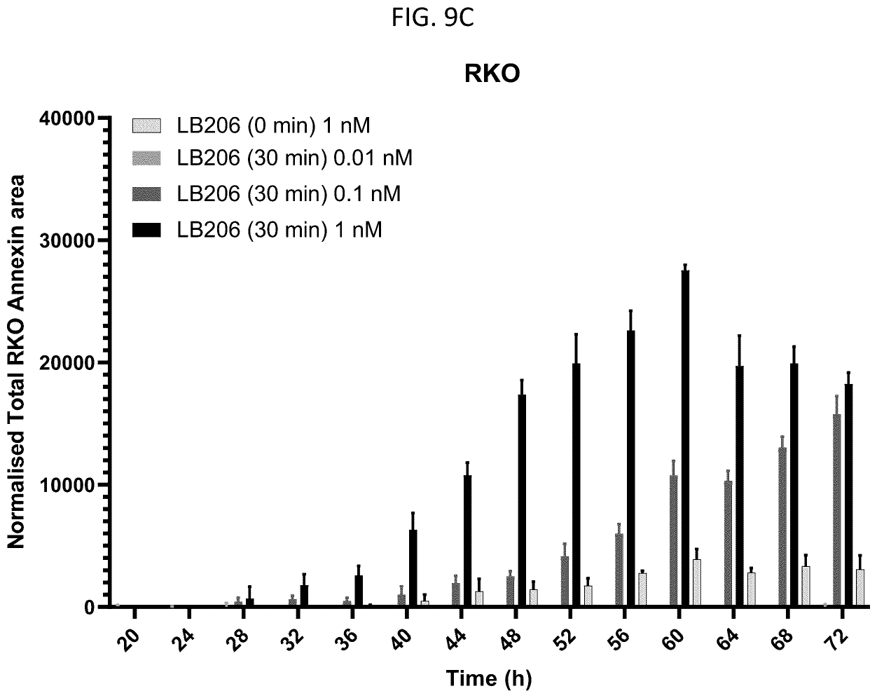

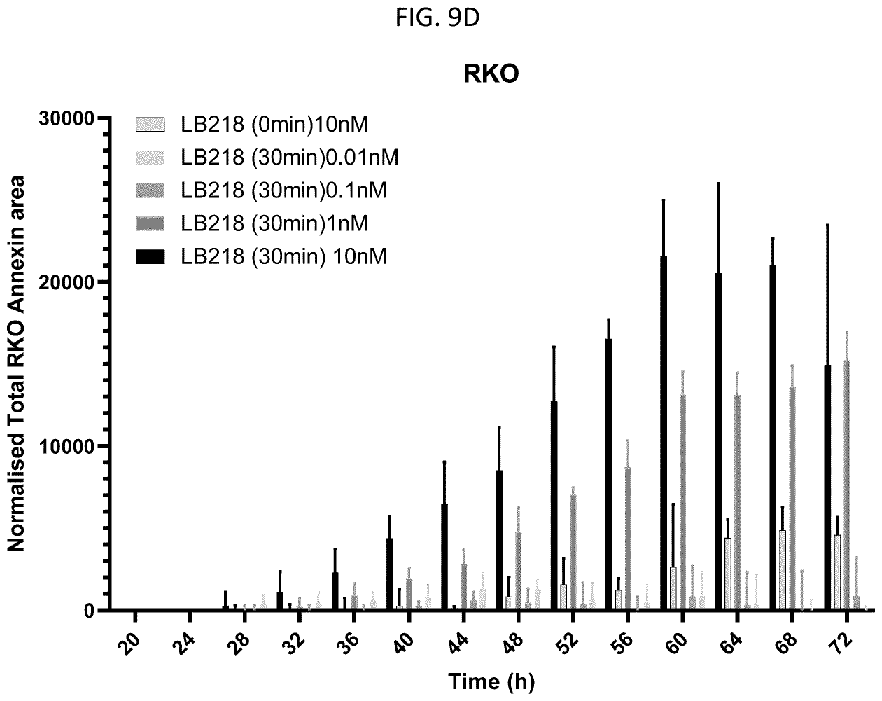

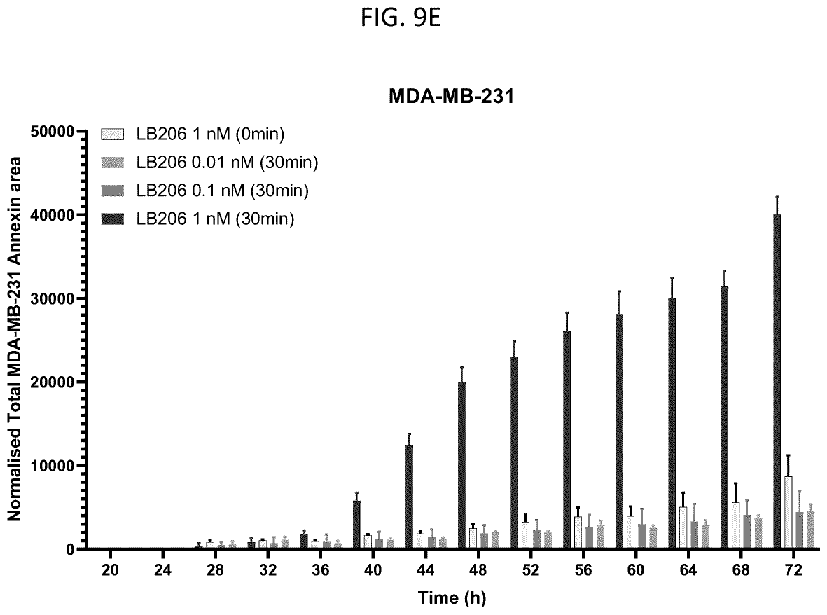

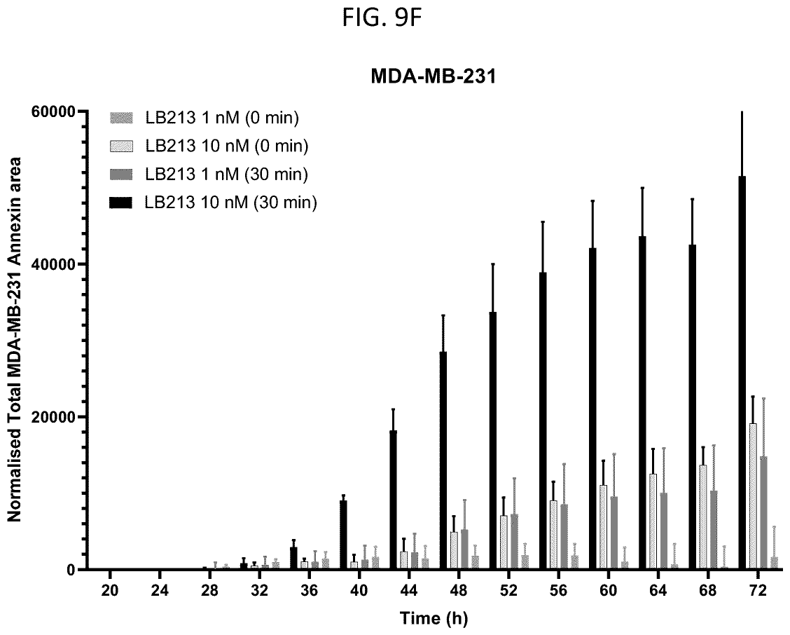

PD-L1+ low (A549) cells were seeded into 96-well plates and grown for approximately 24 h before addition of primary T cells (E:T 5:1) of two different donors and intact or digested LB proteins (LB206 or LB226) at concentrations ranging from 0.01 nM to 10 nM as indicated. Cancer cell killing was assessed using Incucyte® Live-Cell Analysis system by measuring total annexin V area over time and results are shown in FIG. 9A. Intact LB206 induced only low-level background cell killing at 10 nM, while strong Annexin V signal was detected for both 10 nM and 1 nM of digested LB206 protein. 0.1 nM of cleaved LB206 showed very low signals, similar to 10 nM intact LB206 suggesting an apparent 100-fold lower ability to direct T cell killing of undigested LB206. Similarly, intact LB218 did not induce A549 cancer cell killing at 10 nM (FIG. 9B). As observed previously for LB206, strong Annexin V signals over background were detected at 10 nM and 1 nM digested LB218 samples. However, overall induction of cell killing was lower than observed for LB206, confirming that the lower-affinity CD3 variable domains found in LB218 are less potent than those in LB206 in a primary T cell setting.

Next, selected LB proteins were assessed for their ability to induce tumor cell killing by primary T cells in PD-L1+ high RKO cells. As before, cancer cells were seeded for approximately 24 h before addition of primary T cells from two different donors and selected intact or digested LB proteins at concentrations ranging from 0.01 nM to 10 nM or as indicated. Representative results are depicted in FIG. 9C. Cancer cell killing was assessed using Incucyte® Live-Cell Analysis system by measuring total annexin V area over time. In contrast to PD-L1+ low A549 cells, digested LB206 induced strong and sustained cell killing even at 0.1 nM in RKO cells while no or low background was observed for intact LB206 protein at 1 nM. Similarly, intact LB218 did not induce RKO cancer cell killing even at 10 nM (FIG. 9D). As observed previously in A549 cells, strong Annexin V signals over background were detected in 10 nM and 1 nM digested samples for LB218 although onset of cell kill for 1 nM concentration was markedly earlier in RKO cells than for the A549 cells (PD-L1 low) confirming that PD-L1 expression levels determine the ability of LB proteins to induce cell killing.

Next, we assessed selected LB proteins for their ability to induce tumor cell killing by primary T cells in another PD-L1+ high cell line, TNBC MDA-MB-231 cells. As before, tumor cells were seeded and grown for 24 h before addition of primary T cells and selected intact or digested (indicated times) LB proteins at concentrations ranging from 0.01 nM to 10 nM. Cancer cell killing was assessed using Incucyte® Live-Cell Analysis system by measuring total annexin V area over time. Digested LB206 and LB213 induced strong tumor cell killing of MDA-MB-231 cells over time (FIG. 9E-FIG. 9F). As observed previously, LB206 showed most potent tumor cell killing at 10-fold lower concentrations compared to LB213 over time suggesting activation after prolonged exposure to MDA-MB-231 cells.

### Potency of Lead Molecules in Humanized Mouse Model of Triple Negative Breast Cancer (TNBC)

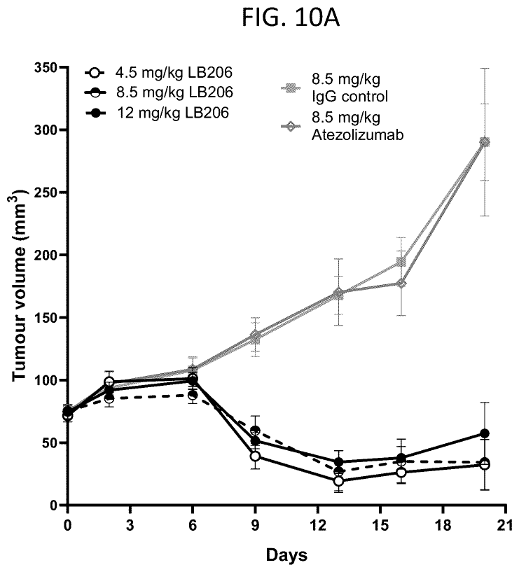

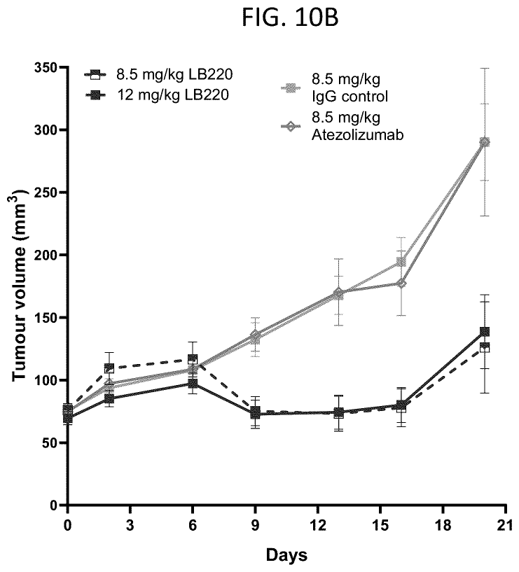

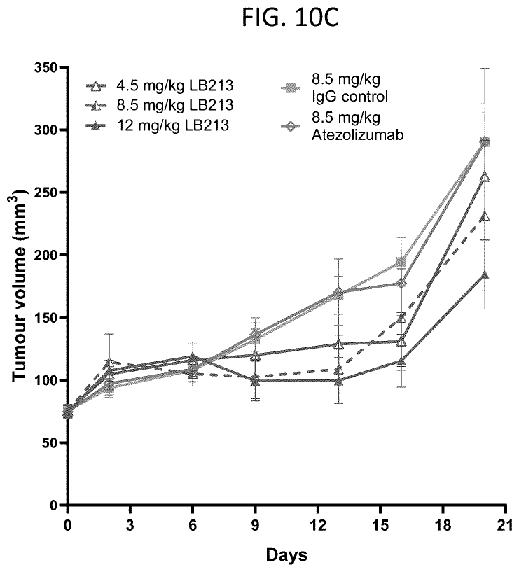

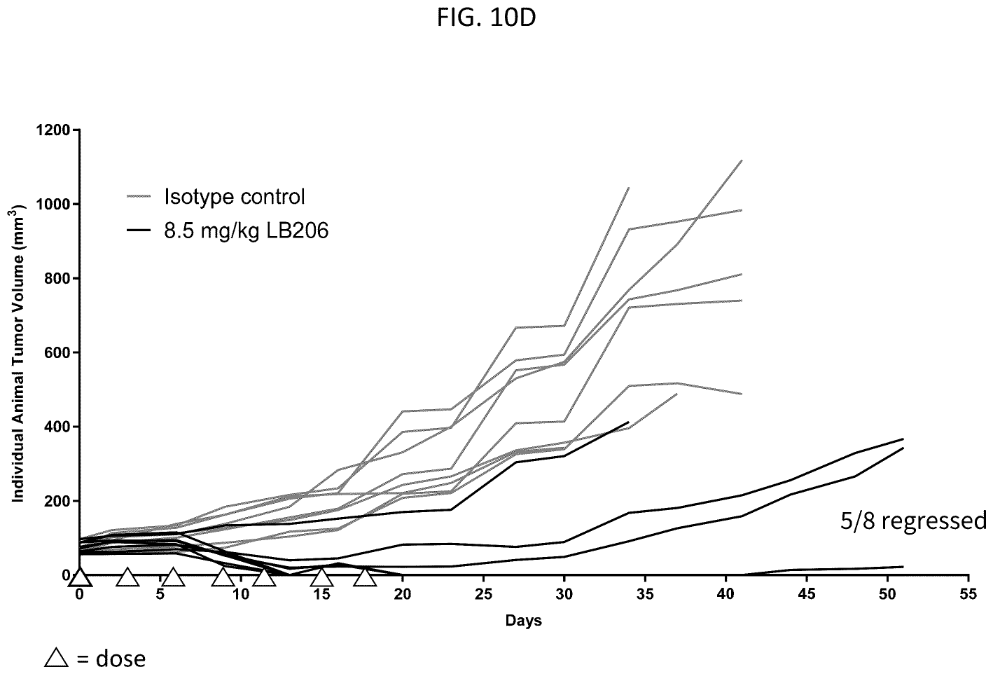

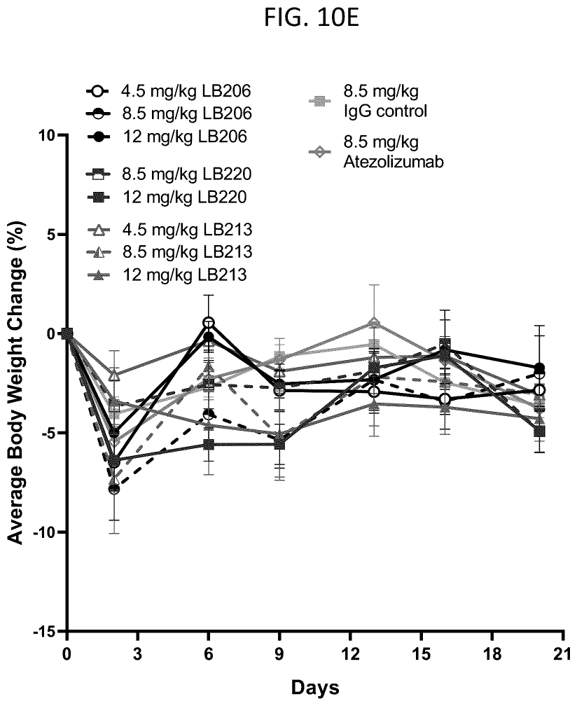

Selected lead LB proteins were assessed for their in vivo performance. MDA-MB-231 cancer cells were subcutaneously inoculated into myeloid cytokine boosted (hGM-CSF+hIL3+hIL4+FLT3L) CD34+ NCG mice. When tumors showed a mean volume of 80 mm3 by caliper measurement, mice were intraperitoneally treated 8×Q3D with either 4.5 mg/kg, 8.5 mg/kg or 12 mg/kg LB proteins, IgG isotype control or atezolizumab as indicated. Tumor volumes were monitored every 3 to 4 days by caliper measurements, mean tumor volumes are shown in FIG. 10. LB206 induced tumor regressions (FIG. 10A, FIG. 10D), LB220 strong tumor growth inhibition (FIG. 10B) and LB213 dose dependent tumor growth inhibition (FIG. 10C) while anti-PD-L1 atezolizumab demonstrated no tumor growth control above isotype control. In the LB206 group, 5/8 animals achieved durable remission (tumor volume=0 mm) over 40 days (FIG. 10D). No toxicity was observed during dosing, with all mice maintaining body weight throughout the experiment, i.e., with no significant changes in comparison to IgG control (FIG. TOE). These data support the hypothesis that the LB protein design facilitates strong potency in highly immune-resistant tumor settings such as MDA-MB-231, which expresses constitutively high levels of PD-L1, without the induction of systemic toxicity. This is a surprising finding, as this model has been run multiple times in previously published studies where immune-engaging bispecific antibodies (including CD3-engaging bispecifics) targeting surface-expressed markers found on MDA-MB-231 cells were dosed at high levels (multiple mgs/kg), with little or no therapeutic success (Del Bano et al., Front. Immunol. 2019, 10:1593; Liu et al., J Immuno Therapy of Cancer 2021, 9:e003468; Kemper et al., Life Sci Alliance 2022, 5(11):e202201481).

It is to be understood that while the invention has been described in conjunction with the preferred specific embodiments thereof, that the foregoing description and the examples that follow are intended to illustrate and not limit the scope of the invention. It will be understood by those skilled in the art that various changes may be made, and equivalents may be substituted without departing from the scope of the invention, and further that other aspects, advantages and modifications will be apparent to those skilled in the art to which the invention pertains. In addition to the embodiments described herein, the present invention contemplates and claims those inventions resulting from the combination of features of the invention cited herein and those of the cited prior art references which complement the features of the present invention. Similarly, it will be appreciated that any described material, feature, or article may be used in combination with any other material, feature, or article, and such combinations are considered within the scope of this invention. The disclosures of each patent, patent application, and publication cited or described in this document are hereby incorporated herein by reference, each in its entirety, for all purposes.

### Numbered Embodiments

Notwithstanding the appended claims, the disclosure sets forth the following numbered embodiments:

Embodiment 1. A protein comprising a first polypeptide chain comprising a heavy chain and a second polypeptide chain comprising a light chain, wherein the heavy chain comprises, in N-terminus to C-terminus order, an anti-PD-L1 heavy chain variable (VH) domain, a first CH1 domain, a first linker, an anti-CD3 VH domain, and a second CH1 domain; and wherein the light chain comprises, in N-terminus to C-terminus order, an anti-PD-L1 light chain variable (VL) domain, a first immunoglobulin light chain constant region, a second linker, an anti-CD3 VL domain, and a second immunoglobulin light chain constant region.

Embodiment 2. A protein comprising a first polypeptide chain comprising a heavy chain and a second polypeptide chain comprising a light chain, wherein the heavy chain comprises, in N-terminus to C-terminus order, an anti-PD-L1 heavy chain variable (VH) domain, a first CH1 domain, a first linker, an anti-CD3 light chain variable (VL) domain, and a first immunoglobulin light chain constant region, and wherein the light chain comprises, in N-terminus to C-terminus order, anti-PD-L1 VL domain, a second immunoglobulin light chain constant region, a second linker, an anti-CD3 VH domain, and a second CH1 domain.

Embodiment 3. The protein of embodiment 1 or 2, wherein the heavy chain comprises in N-terminus to C-terminus order, the anti-PD-L1 VH domain, the first CH1 domain, the first linker, the anti-CD3 VH domain, the second CH1 domain, a hinge, a CH2 domain, and a CH3 domain.

Embodiment 4. The protein of any one of embodiments 1-3, wherein the protein further comprises a third polypeptide chain comprising a hinge, a CH2 domain, and a CH3 domain.

Embodiment 5. The protein of embodiment 4, wherein the third polypeptide chain comprises the amino acid sequence of SEQ ID NO: 124.

Embodiment 6. The protein of embodiment 1, wherein the protein further comprises a moiety that provides half-life extension.

Embodiment 7. The protein of embodiment 6, wherein the moiety that provides half-life extension is polyethylene glycol (PEG) or an albumin binding domain.

Embodiment 8. A protein comprising a first polypeptide chain comprising a heavy chain and a second polypeptide chain comprising a light chain, wherein the heavy chain comprises, in N-terminus to C-terminus order, an anti-PD-L1 heavy chain variable (VH) domain, a first CH1 domain, a first linker, an anti-CD3 VH domain, a second CH1 domain, a first hinge, a first CH2 domain, and a first CH3 domain; and
wherein the light chain comprises, in N-terminus to C-terminus order, an anti-PD-L1 light chain variable (VL) domain, a first immunoglobulin light chain constant region, a second linker, an anti-CD3 VL domain, a second immunoglobulin light chain constant region, a second hinge, a second CH2 domain, and a second CH3 domain.

Embodiment 9. The protein of any one of embodiments 1-8, wherein the first linker comprises the amino acid sequence of any one of SEQ ID NOs: 1-12.

Embodiment 10. The protein of any one of embodiments 1-9, wherein the second linker comprises the amino acid sequence of any one of SEQ ID NOs: 1-12.

Embodiment 11. The protein of any one of embodiments 1-10, wherein the anti-PD-L1 VH domain comprises a HCDR1 comprising the amino acid sequence of SEQ ID NO: 20, a HCDR2 comprising the amino acid sequence of SEQ ID NO: 21, and a HCDR3 comprising the amino acid sequence of SEQ ID NO: 22; and wherein the anti-PD-L1 VL domain comprises a LCDR1 comprising the amino acid sequence of SEQ ID NO: 23, a LCDR2 comprising the amino acid sequence of SEQ ID NO: 24, and a LCDR3 comprising the amino acid sequence of SEQ ID NO: 25.

Embodiment 12. The protein of any one of embodiments 1-11, wherein the anti-CD3 VH domain comprises a HCDR1 comprising the amino acid sequence of SEQ ID NO: 26, a HCDR2 comprising the amino acid sequence of SEQ ID NO: 27, and a HCDR3 comprising the amino acid sequence of SEQ ID NO: 29; and wherein the anti-CD3 VL domain comprises a LCDR1 comprising the amino acid sequence of SEQ ID NO: 30, a LCDR2 comprising the amino acid sequence of SEQ ID NO: 31, and a LCDR3 comprising the amino acid sequence of SEQ ID NO: 32.

Embodiment 13. The protein of any one of embodiments 1-11, wherein the anti-CD3 VH domain comprises a HCDR1 comprising the amino acid sequence of SEQ ID NO: 26, a HCDR2 comprising the amino acid sequence of SEQ ID NO: 28, and a HCDR3 comprising the amino acid sequence of SEQ ID NO: 29; and wherein the anti-CD3 VL domain comprises a LCDR1 comprising the amino acid sequence of SEQ ID NO: 30, a LCDR2 comprising the amino acid sequence of SEQ ID NO: 31, and a LCDR3 comprising the amino acid sequence of SEQ ID NO: 32.

Embodiment 14. The protein of any one of embodiments 1-10, wherein the anti-PD-L1 VH domain comprises a HCDR1 comprising the amino acid sequence of SEQ ID NO: 20, a HCDR2 comprising the amino acid sequence of SEQ ID NO: 21, and a HCDR3 comprising the amino acid sequence of SEQ ID NO: 22; wherein the anti-PD-L1 VL domain comprises a LCDR1 comprising the amino acid sequence of SEQ ID NO: 23, a LCDR2 comprising the amino acid sequence of SEQ ID NO: 24, and a LCDR3 comprising the amino acid sequence of SEQ ID NO: 25; wherein the anti-CD3 VH domain comprises a HCDR1 comprising the amino acid sequence of SEQ ID NO: 26, a HCDR2 comprising the amino acid sequence of SEQ ID NO: 27, and a HCDR3 comprising the amino acid sequence of SEQ ID NO: 29; and wherein the anti-CD3 VL domain comprises a LCDR1 comprising the amino acid sequence of SEQ ID NO: 30, a LCDR2 comprising the amino acid sequence of SEQ ID NO: 31, and a LCDR3 comprising the amino acid sequence of SEQ ID NO: 32.

Embodiment 15. The protein of any one of embodiments 1-10, wherein the anti-PD-L1 VH domain comprises a HCDR1 comprising the amino acid sequence of SEQ ID NO: 20, a HCDR2 comprising the amino acid sequence of SEQ ID NO: 21, and a HCDR3 comprising the amino acid sequence of SEQ ID NO: 22;
wherein the anti-PD-L1 VL domain comprises a LCDR1 comprising the amino acid sequence of SEQ ID NO: 23, a LCDR2 comprising the amino acid sequence of SEQ ID NO: 24, and a LCDR3 comprising the amino acid sequence of SEQ ID NO: 25;
   wherein the anti-CD3 VH domain comprises a HCDR1 comprising the amino acid sequence of SEQ ID NO: 26, a HCDR2 comprising the amino acid sequence of SEQ ID NO: 28, and a HCDR3 comprising the amino acid sequence of SEQ ID NO: 29; and wherein the anti-CD3 VL domain comprises a LCDR1 comprising the amino acid sequence of SEQ ID NO: 30, a LCDR2 comprising the amino acid sequence of SEQ ID NO: 31, and a LCDR3 comprising the amino acid sequence of SEQ ID NO: 32.

Embodiment 16. The protein of any one of embodiments 1-15, wherein the anti-PD-L1 VH domain comprises the amino acid sequence of any one of SEQ ID NOs: 33 and 54-107.

Embodiment 17. The protein of any one of embodiments 1-16, wherein the anti-PD-L1 VL domain comprises the amino acid sequence of SEQ ID NO: 34.

Embodiment 18. The protein of any one of embodiments 1-17, wherein the anti-PD-L1 VH domain comprises the amino acid sequence of any one of SEQ ID NOs: 33 and 54-107, and the anti-PD-L1 VL domain comprises the amino acid sequence of SEQ ID NO: 34.

Embodiment 19. The protein of any one of embodiments 1-17, wherein the anti-CD3 VH domain comprises the amino acid sequence of any one of SEQ ID NOs: 44-48.

Embodiment 20. The protein of any one of embodiments 1-19, wherein the anti-CD3 VL domain comprises the amino acid sequence of any one of SEQ ID NOs: 37-42.

Embodiment 21. The protein of any one of embodiments 1-20, wherein the heavy chain comprises the amino acid sequence of any one of SEQ ID NOs: 116-123, 129-132, 137-141, 142, 144, 146, 147, 148, 150, and 152.

Embodiment 22. The protein of any one of embodiments 1-21, wherein the light chain comprises the amino acid sequence of any one of SEQ ID NOs: 53, 108-115, 125-128, 133-136, 143, 145, 151, and 153.

Embodiment 23. The protein of embodiment 4, wherein:
(a) the heavy chain comprises the amino acid sequence of SEQ ID NO: 122, the light chain comprises the amino acid sequence of SEQ ID NO: 114, and the third polypeptide chain comprises the amino acid sequence of SEQ ID NO: 124;
(b) the heavy chain comprises the amino acid sequence of SEQ ID NO: 123, the light chain comprises the amino acid sequence of SEQ ID NO: 115, and the third polypeptide chain comprises the amino acid sequence of SEQ ID NO: 124;
(c) the heavy chain comprises the amino acid sequence of SEQ ID NO: 116, the light chain comprises the amino acid sequence of SEQ ID NO: 108, and the third polypeptide chain comprises the amino acid sequence of SEQ ID NO: 124;
(d) the heavy chain comprises the amino acid sequence of SEQ ID NO: 117, the light chain comprises the amino acid sequence of SEQ ID NO: 109, and the third polypeptide chain comprises the amino acid sequence of SEQ ID NO: 124;
(e) the heavy chain comprises the amino acid sequence of SEQ ID NO: 118, the light chain comprises the amino acid sequence of SEQ ID NO: 108, and the third polypeptide chain comprises the amino acid sequence of SEQ ID NO: 124;
(f) the heavy chain comprises the amino acid sequence of SEQ ID NO: 119, the light chain comprises the amino acid sequence of SEQ ID NO: 109, and the third polypeptide chain comprises the amino acid sequence of SEQ ID NO: 124;
(g) the heavy chain comprises the amino acid sequence of SEQ ID NO: 120, the light chain comprises the amino acid sequence of SEQ ID NO: 108, and the third polypeptide chain comprises the amino acid sequence of SEQ ID NO: 124;
(h) the heavy chain comprises the amino acid sequence of SEQ ID NO: 116, the light chain comprises the amino acid sequence of SEQ ID NO: 110, and the third polypeptide chain comprises the amino acid sequence of SEQ ID NO: 124;
(i) the heavy chain comprises the amino acid sequence of SEQ ID NO: 118, the light chain comprises the amino acid sequence of SEQ ID NO: 110, and the third polypeptide chain comprises the amino acid sequence of SEQ ID NO: 124;
(j) the heavy chain comprises the amino acid sequence of SEQ ID NO: 120, the light chain comprises the amino acid sequence of SEQ ID NO: 110, and the third polypeptide chain comprises the amino acid sequence of SEQ ID NO: 124;
(k) the heavy chain comprises the amino acid sequence of SEQ ID NO: 116, the light chain comprises the amino acid sequence of SEQ ID NO: 111, and the third polypeptide chain comprises the amino acid sequence of SEQ ID NO: 124;
(l) the heavy chain comprises the amino acid sequence of SEQ ID NO: 118, the light chain comprises the amino acid sequence of SEQ ID NO: 111, and the third polypeptide chain comprises the amino acid sequence of SEQ ID NO: 124;
(m) the heavy chain comprises the amino acid sequence of SEQ ID NO: 120, the light chain comprises the amino acid sequence of SEQ ID NO: 111, and the third polypeptide chain comprises the amino acid sequence of SEQ ID NO: 124;
(n) the heavy chain comprises the amino acid sequence of SEQ ID NO: 116, the light chain comprises the amino acid sequence of SEQ ID NO: 112, and the third polypeptide chain comprises the amino acid sequence of SEQ ID NO: 124;
(o) the heavy chain comprises the amino acid sequence of SEQ ID NO: 116, the light chain comprises the amino acid sequence of SEQ ID NO: 113, and the third polypeptide chain comprises the amino acid sequence of SEQ ID NO: 124;
(p) the heavy chain comprises the amino acid sequence of SEQ ID NO: 121, the light chain comprises the amino acid sequence of SEQ ID NO: 108, and the third polypeptide chain comprises the amino acid sequence of SEQ ID NO: 124;
(q) the heavy chain comprises the amino acid sequence of SEQ ID NO: 131, the light chain comprises the amino acid sequence of SEQ ID NO: 127, and the third polypeptide chain comprises the amino acid sequence of SEQ ID NO: 124; or
(r) the heavy chain comprises the amino acid sequence of SEQ ID NO: 120, the light chain comprises the amino acid sequence of SEQ ID NO: 127, and the third polypeptide chain comprises the amino acid sequence of SEQ ID NO: 124.

Embodiment 24. The protein of any one of embodiments 2-23, wherein the heavy chain comprises an IgG, IgE, IgM, IgD, IgA, or IgY constant region.

Embodiment 25. The protein of any one of embodiments 2-23, wherein the heavy chain comprises an IgG1, IgG2, IgG3, IgG4, IgA1 or IgA2 constant region.

Embodiment 26. The protein of any one of embodiments 2-23, wherein the heavy chain comprises an immunologically inert constant region.

Embodiment 27. The protein of any one of embodiments 2-23, wherein the heavy chain comprises a wild-type human IgG1 constant region, a human IgG1 constant region comprising the amino acid substitutions L234A, L235A and G237A, a wild-type human IgG2 constant region, a wild-type human IgG4 constant region, or a human IgG4 constant region comprising the amino acid substitution S228P, wherein numbering is according to the EU index as in Kdbat.

Embodiment 28. An immunoconjugate comprising the protein of any one of embodiments 1-27, linked to a therapeutic agent.

Embodiment 29. The immunoconjugate of embodiment 28, wherein the therapeutic agent is a cytotoxin, a radioisotope, a chemotherapeutic agent, an immunomodulatory agent, a cytostatic enzyme, a cytolytic enzyme, a therapeutic nucleic acid, an anti-angiogenic agent, an anti-proliferative agent, or a pro-apoptotic agent.

Embodiment 30. A pharmaceutical composition comprising the protein of any one of embodiments 1-27 or the immunoconjugate of embodiment 28 or 29, and a pharmaceutically acceptable carrier.

Embodiment 31. A nucleic acid molecule encoding
(a) the heavy chain amino acid sequence;
(b) the light chain amino acid sequence; or
(c) both the heavy chain and the light chain amino acid sequences of the protein of any one of embodiments 1-27.

Embodiment 32. An expression vector comprising the nucleic acid molecule of embodiment 31.

Embodiment 33. A recombinant host cell comprising the nucleic acid molecule of embodiment 31 or the expression vector of embodiment 32.

Embodiment 34. A method of producing a protein, the method comprising: culturing the recombinant host cell of embodiment 33 under conditions whereby the nucleic acid molecule is expressed, thereby producing the protein; and isolating the protein from the host cell or culture.

Embodiment 35. A method for enhancing an anti-cancer immune response in a subject, the method comprising administering to the subject a therapeutically effective amount of the protein of any one of embodiments 1-27, the immunoconjugate of embodiment 28 or 29, or the pharmaceutical composition of embodiment 30.

Embodiment 36. A method for treating cancer in a subject, the method comprising administering to the subject a therapeutically effective amount of the protein of any one of embodiments 1-27, the immunoconjugate of embodiment 28 or 29, or the pharmaceutical composition of embodiment 30.

Embodiment 37. A method for ameliorating a symptom of cancer in a subject, the method comprising administering to the subject a therapeutically effective amount of the protein of any one of embodiments 1-27, the immunoconjugate of embodiment 28 or 29, or the pharmaceutical composition of embodiment 30.

Embodiment 38. The method of any one of embodiments 35-37, wherein the cancer is gastrointestinal stromal cancer (GIST), pancreatic cancer, skin cancer, melanoma, breast cancer, lung cancer, bronchial cancer, colorectal cancer, prostate cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of the oral cavity or pharynx, liver cancer, kidney cancer, renal cell carcinoma, testicular cancer, biliary tract cancer, small bowel cancer, appendix cancer, salivary gland cancer, thyroid cancer, adrenal gland cancer, osteosarcoma, chondrosarcoma, or cancer of hematological tissues.

Embodiment 39. A protein of any one of embodiments 1-27, the immunoconjugate of embodiment 28 or 29, or the pharmaceutical composition of embodiment 30, for use in enhancing an anti-cancer immune response in a subject.

Embodiment 40. A protein of any one of embodiments 1-27, the immunoconjugate of embodiment 28 or 29, or the pharmaceutical composition of embodiment 30, for use in treating cancer in a subject.

Embodiment 41. A protein of any one of embodiments 1-27, the immunoconjugate of embodiment 28 or 29, or the pharmaceutical composition of embodiment 30, for use in ameliorating a symptom of cancer in a subject.

Embodiment 42. A protein, an immunoconjugate, or a pharmaceutical composition for use according to any one of embodiments 39-42, wherein the cancer is gastrointestinal stromal cancer (GIST), pancreatic cancer, skin cancer, melanoma, breast cancer, lung cancer, bronchial cancer, colorectal cancer, prostate cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of the oral cavity or pharynx, liver cancer, kidney cancer, renal cell carcinoma, testicular cancer, biliary tract cancer, small bowel cancer, appendix cancer, salivary gland cancer, thyroid cancer, adrenal gland cancer, osteosarcoma, chondrosarcoma, or cancer of hematological tissues.

1.-42. (canceled)

43. A protein comprising a first polypeptide chain comprising a heavy chain and a second polypeptide chain comprising a light chain, wherein:
(a) the heavy chain comprises, in N-terminus to C-terminus order, an anti-PD-L1 heavy chain variable (VH) domain, a first CH1 domain, a first linker, an anti-CD3 heavy chain variable (VH) domain, and a second CH1 domain, and
the light chain comprises, in N-terminus to C-terminus order, an anti-PD-L1 light chain variable (VL) domain, a first immunoglobulin light chain constant region, a second linker, an anti-CD3 light chain variable (VL) domain, and a second immunoglobulin light chain constant region; or
(b) the heavy chain comprises, in N-terminus to C-terminus order, an anti-PD-L1 heavy chain variable (VH) domain, a first CH1 domain, a first linker, an anti-CD3 light chain variable (VL) domain, and a first immunoglobulin light chain constant region, and the light chain comprises, in N-terminus to C-terminus order, an anti-PD-L1 light chain variable (VL) domain, a second immunoglobulin light chain constant region, a second linker, an anti-CD3 heavy chain variable (VH) domain, and a second CH1 domain.

44. The protein of claim 43, wherein the protein further comprises a third polypeptide chain comprising, in N-terminus to C-terminus order, a first hinge, a first CH2 domain, and a first CH3 domain.

45. The protein of claim 44, wherein the C-terminus of the first polypeptide chain is operably linked to the N-terminus of the third polypeptide chain.

46. The protein of claim 45, wherein the third polypeptide chain comprises the amino acid sequence of SEQ ID NO: 124.

47. The protein of claim 43, wherein the first linker or the second linker comprises an amino acid sequence having at least 95% sequence identity to the amino acid sequence of any one of SEQ ID NOs: 1-12.

48. The protein of claim 43, wherein the anti-PD-L1 heavy chain variable (VH) domain comprises a heavy chain complementarity region 1 (HCDR1) comprising the amino acid sequence of SEQ ID NO: 20, a heavy chain complementarity region 2 (HCDR2) comprising the amino acid sequence of SEQ ID NO: 21, and a heavy chain complementarity region 3 (HCDR3) comprising the amino acid sequence of SEQ ID NO: 22; and wherein the anti-PD-L1 light chain variable (VL) domain comprises a light chain complementarity region 1 (LCDR1) comprising the amino acid sequence of SEQ ID NO: 23, a light chain complementarity region 2 (LCDR2) comprising the amino acid sequence of SEQ ID NO: 24, and a light chain complementarity region 3 (LCDR3) comprising the amino acid sequence of SEQ ID NO: 25.

49. The protein of claim 43, wherein:
(a) the anti-CD3 heavy chain variable (VH) domain comprises a HCDR1 comprising the amino acid sequence of SEQ ID NO: 26, a HCDR2 comprising the amino acid sequence of SEQ ID NO: 27, and a HCDR3 comprising the amino acid sequence of SEQ ID NO: 29; and the anti-CD3 light chain variable (VL) domain comprises a LCDR1 comprising the amino acid sequence of SEQ ID NO: 30, a LCDR2 comprising the amino acid sequence of SEQ ID NO: 31, and a LCDR3 comprising the amino acid sequence of SEQ ID NO: 32; or
(b) the anti-CD3 heavy chain variable (VH) domain comprises a HCDR1 comprising the amino acid sequence of SEQ ID NO: 26, a HCDR2 comprising the amino acid sequence of SEQ ID NO: 28, and a HCDR3 comprising the amino acid sequence of SEQ ID NO: 29; and
the anti-CD3 light chain variable (VL) domain comprises a LCDR1 comprising the amino acid sequence of SEQ ID NO: 30, a LCDR2 comprising the amino acid sequence of SEQ ID NO: 31, and a LCDR3 comprising the amino acid sequence of SEQ ID NO: 32.

50. The protein of claim 43, wherein the anti-PD-L1 heavy chain variable (VH) domain comprises an amino acid sequence that is at least 90% identical to the amino acid sequence of any one of SEQ ID NOs: 33 and 54-107.

51. The protein of claim 43, wherein the anti-PD-L1 light chain variable (VL) domain comprises an amino acid sequence that is at least 90% identical to the amino acid sequence of SEQ ID NO: 34.

52. The protein of claim 43, wherein the anti-CD3 heavy chain variable (VH) domain comprises an amino acid sequence that is at least 90% identical to the amino acid sequence of any one of SEQ ID NOs: 44-48.

53. The protein of claim 43, wherein the anti-CD3 light chain variable (VL) domain comprises an amino acid sequence that is at least 90% identical to the amino acid sequence of any one of SEQ ID NOs: 37-42.

54. The protein of claim 43, wherein the heavy chain comprises an amino acid sequence that is at least 90% identical to the amino acid sequence of any one of SEQ ID NOs: 116-123, 129-132, 137-141, 142, 144, 146, 147, 148, 150, and 152.

55. The protein of claim 43, wherein the light chain comprises an amino acid sequence that is at least 90% identical to the amino acid sequence of any one of SEQ ID NOs: 53, 108-115, 125-128, 133-136, 143, 145, 151, and 153.

56. The protein of claim 44, wherein:
(a) the heavy chain comprises the amino acid sequence of SEQ ID NO: 122, the light chain comprises the amino acid sequence of SEQ ID NO: 114, and the third polypeptide chain comprises the amino acid sequence of SEQ ID NO: 124;
(b) the heavy chain comprises the amino acid sequence of SEQ ID NO: 123, the light chain comprises the amino acid sequence of SEQ ID NO: 115, and the third polypeptide chain comprises the amino acid sequence of SEQ ID NO: 124;
(c) the heavy chain comprises the amino acid sequence of SEQ ID NO: 116, the light chain comprises the amino acid sequence of SEQ ID NO: 108, and the third polypeptide chain comprises the amino acid sequence of SEQ ID NO: 124;
(d) the heavy chain comprises the amino acid sequence of SEQ ID NO: 117, the light chain comprises the amino acid sequence of SEQ ID NO: 109, and the third polypeptide chain comprises the amino acid sequence of SEQ ID NO: 124;
(e) the heavy chain comprises the amino acid sequence of SEQ ID NO: 118, the light chain comprises the amino acid sequence of SEQ ID NO: 108, and the third polypeptide chain comprises the amino acid sequence of SEQ ID NO: 124;
(f) the heavy chain comprises the amino acid sequence of SEQ ID NO: 119, the light chain comprises the amino acid sequence of SEQ ID NO: 109, and the third polypeptide chain comprises the amino acid sequence of SEQ ID NO: 124;
(g) the heavy chain comprises the amino acid sequence of SEQ ID NO: 120, the light chain comprises the amino acid sequence of SEQ ID NO: 108, and the third polypeptide chain comprises the amino acid sequence of SEQ ID NO: 124;
(h) the heavy chain comprises the amino acid sequence of SEQ ID NO: 116, the light chain comprises the amino acid sequence of SEQ ID NO: 110, and the third polypeptide chain comprises the amino acid sequence of SEQ ID NO: 124;
(i) the heavy chain comprises the amino acid sequence of SEQ ID NO: 118, the light chain comprises the amino acid sequence of SEQ ID NO: 110, and the third polypeptide chain comprises the amino acid sequence of SEQ ID NO: 124;
(j) the heavy chain comprises the amino acid sequence of SEQ ID NO: 120, the light chain comprises the amino acid sequence of SEQ ID NO: 110, and the third polypeptide chain comprises the amino acid sequence of SEQ ID NO: 124;
(k) the heavy chain comprises the amino acid sequence of SEQ ID NO: 116, the light chain comprises the amino acid sequence of SEQ ID NO: 111, and the third polypeptide chain comprises the amino acid sequence of SEQ ID NO: 124;
(l) the heavy chain comprises the amino acid sequence of SEQ ID NO: 118, the light chain comprises the amino acid sequence of SEQ ID NO: 111, and the third polypeptide chain comprises the amino acid sequence of SEQ ID NO: 124;
(m) the heavy chain comprises the amino acid sequence of SEQ ID NO: 120, the light chain comprises the amino acid sequence of SEQ ID NO: 111, and the third polypeptide chain comprises the amino acid sequence of SEQ ID NO: 124;
(n) the heavy chain comprises the amino acid sequence of SEQ ID NO: 116, the light chain comprises the amino acid sequence of SEQ ID NO: 112, and the third polypeptide chain comprises the amino acid sequence of SEQ ID NO: 124;
(o) the heavy chain comprises the amino acid sequence of SEQ ID NO: 116, the light chain comprises the amino acid sequence of SEQ ID NO: 113, and the third polypeptide chain comprises the amino acid sequence of SEQ ID NO: 124;
(p) the heavy chain comprises the amino acid sequence of SEQ ID NO: 121, the light chain comprises the amino acid sequence of SEQ ID NO: 108, and the third polypeptide chain comprises the amino acid sequence of SEQ ID NO: 124;
(q) the heavy chain comprises the amino acid sequence of SEQ ID NO: 131, the light chain comprises the amino acid sequence of SEQ ID NO: 127, and the third polypeptide chain comprises the amino acid sequence of SEQ ID NO: 124; or
(r) the heavy chain comprises the amino acid sequence of SEQ ID NO: 120, the light chain comprises the amino acid sequence of SEQ ID NO: 127, and the third polypeptide chain comprises the amino acid sequence of SEQ ID NO: 124.

57. The protein of claim 43, wherein the heavy chain comprises an IgG, IgE, IgM, IgD, IgA, or IgY constant region.

58. The protein of claim 43, wherein the heavy chain comprises an immunologically inert constant region.

59. The protein of claim 43, wherein the heavy chain comprises a wild-type human IgG1 constant region, a human IgG1 constant region comprising the amino acid substitutions L234A, L235A and G237A, a wild-type human IgG2 constant region, a wild-type human IgG4 constant region, or a human IgG4 constant region comprising the amino acid substitution S228P, wherein numbering is according to the EU index as in Kdbat.

60. A pharmaceutical composition comprising the protein of claim 43, and a pharmaceutically acceptable carrier.

61. A method of treating cancer in a subject, the method comprising administering to the subject a therapeutically effective amount of the protein of claim 43, or the pharmaceutical composition of claim 60.

62. The method of claim 61, wherein the cancer is gastrointestinal stromal cancer (GIST), pancreatic cancer, skin cancer, melanoma, breast cancer, lung cancer, bronchial cancer, colorectal cancer, prostate cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of the oral cavity or pharynx, liver cancer, kidney cancer, renal cell carcinoma, testicular cancer, biliary tract cancer, small bowel cancer, appendix cancer, salivary gland cancer, thyroid cancer, adrenal gland cancer, osteosarcoma, chondrosarcoma, or cancer of hematological tissues.


SEQUENCE LISTING (153 sequences)

SEQ ID NO: 1 - protein, 11 aa, synthetic construct
GPAPELLGGGS

SEQ ID NO: 2 - protein, 11 aa, synthetic construct
GPAPLGLGGGS

SEQ ID NO: 3 - protein, 12 aa, synthetic construct
GPAPELLGGGGS

SEQ ID NO: 4 - protein, 12 aa, synthetic construct
GPAPLGLGGGGS

SEQ ID NO: 5 - protein, 11 aa, synthetic construct
GPAPLGLRGGS

SEQ ID NO: 6 - protein, 12 aa, synthetic construct
GPAPLGLRGGGS

SEQ ID NO: 7 - protein, 5 aa, synthetic construct
GGGGS

SEQ ID NO: 8 - protein, 10 aa, synthetic construct
GGGGSGGGGS

SEQ ID NO: 9 - protein, 15 aa, synthetic construct
GGGGSGGGGSGGGGS

SEQ ID NO: 10 - protein, 7 aa, synthetic construct
GPAPELL

SEQ ID NO: 11 - protein, 13 aa, synthetic construct
PPCPAPELLGGGS

SEQ ID NO: 12 - protein, 13 aa, synthetic construct
PPCPAPLGLGGGS

SEQ ID NO: 13 - protein, 103 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 14 - protein, 330 aa, Homo sapiens [sequence omitted]

SEQ ID NO: 15 - protein, 107 aa, Homo sapiens [sequence omitted]

SEQ ID NO: 16 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 18 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 5 aa, synthetic construct
TYAIS

SEQ ID NO: 21 - protein, 17 aa, synthetic construct
GIIPIFGKAHYAQKFQG

SEQ ID NO: 22 - protein, 14 aa, synthetic construct
KFHFVSGSPFGMDV

SEQ ID NO: 23 - protein, 11 aa, synthetic construct
RASQSVSSYLA

SEQ ID NO: 24 - protein, 7 aa, synthetic construct
DASNRAT

SEQ ID NO: 25 - protein, 8 aa, synthetic construct
QQRSNWPT

SEQ ID NO: 26 - protein, 5 aa, synthetic construct
TYAMN

SEQ ID NO: 27 - protein, 19 aa, synthetic construct
RIRSKYNNYATYYADSVKD

SEQ ID NO: 28 - protein, 19 aa, synthetic construct
RIRSKYNNYATYYADSVKG

SEQ ID NO: 29 - protein, 14 aa, synthetic construct
HGNFGNSYVSWFAY

SEQ ID NO: 30 - protein, 14 aa, synthetic construct
RSSTGAVTTSNYAN

SEQ ID NO: 31 - protein, 7 aa, synthetic construct
GTNKRAP

SEQ ID NO: 32 - protein, 9 aa, synthetic construct
ALWYSNLWV

SEQ ID NO: 33 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 34 - protein, 106 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 35 - protein, 109 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRIT

SEQ ID NO: 36 - protein, 109 aa, Macaca fascicularis
FTVTVPKDLYVVEYGSNMTIECKFPVEKQLDLTSLIVYWEMEDKNIIQFVHGEEDLKVQHSNYRQRAQLLKDQLSLGNAALRITDVKLQDAGVYRCMISYGGADYKRIT

SEQ ID NO: 37 - protein, 109 aa, synthetic construct
QAVVTQEPSLTVSPGGTVTLTCRSSTGAVTTSNYANWVQQKPDHLPRALIGGTNKRAPGTPARFSGSLLGDKAALTLSGVQPEDEAEYYCALWYSNLWVFGGGTKLTVL

SEQ ID NO: 38 - protein, 109 aa, synthetic construct
QTVVTQEPSLTVSPGGTVTLTCRSSTGAVTTSNYPNWVQQKPGQAPRALIGGTNKRAPGTPARFSGSLLGGKAALTLSGVQPEDEAEYYCALWYSNLWVFGGGTKLTVL

SEQ ID NO: 39 - protein, 109 aa, synthetic construct
QAVVTQEPSLTVSPGGTVTLTCRSSTGAVTTSNYANWVQQKPDHLPRALIGGTNKRAPGVPARFSGSLLGDKAALTISGAQPEDEAIYYCALWYSNLWVFGGGTKLTVL

SEQ ID NO: 40 - protein, 109 aa, synthetic construct
QAVVTQEPSLTVSPGGTVTLTCRSSTGAVTSGYYANWVQQKPDHLPRALIGGTNKRAPGTPARFSGSLLGDKAALTLSGVQPEDEAEYYCALWYSNLWVFGGGTKLTVL

SEQ ID NO: 41 - protein, 109 aa, synthetic construct
QTVVTQEPSLTVSPGGTVTLTCRSSTGAVTSGYYPNWVQQKPGQAPRALIGGTNKRAPGTPARFSGSLLGGKAALTLSGVQPEDEAEYYCALWYSNLWVFGGGTKLTVL

SEQ ID NO: 42 - protein, 109 aa, synthetic construct
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 43 - protein, 109 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 44 - protein, 125 aa, synthetic construct
EVQLVESGGGLVQPGGSLRLSCAASGFTFNTYAMNWVRQAPGKGLEWVGRIRSKYNNYATYYADSVKDRFTISRDDSQSILYLQMNSLKTEDTAVYYCVRHGNFGNSYVSWFAYWGQGTLVTVSS

SEQ ID NO: 45 - protein, 125 aa, synthetic construct
EVQLVESGGGLVQPGGSLRLSCAASGFTFNTYAMNWVRQAPGKGLEWVARIRSKYNNYATYYADSVKDRFTISRDDSQSILYLQMNSLKTEDTAVYYCVRHGNFGNSYVSWFAYWGQGTLVTVSS

SEQ ID NO: 46 - protein, 125 aa, synthetic construct
EVQLVESGGGLVQPGGSLRLSCAASGFTFNTYAMNWVRQAPGKGLEWVGRIRSKYNNYATYYADSVKGRFTISRDDSKNSLYLQMNSLKTEDTAVYYCARHGNFGNSYVSWFAYWGQGTLVTVSS

SEQ ID NO: 47 - protein, 125 aa, synthetic construct
EVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCARHGNFGNSYVSWFAYWGQGTLVTVSS

SEQ ID NO: 48 - protein, 125 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 49 - protein, 125 aa, synthetic construct
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSS

SEQ ID NO: 50 - skipped

SEQ ID NO: 51 - protein, 17 aa, synthetic construct
  VARIANT 1: note = G, N or H
  VARIANT 3: note = I or F
  VARIANT 5: note = I, K, W, L, Y, Q or R
  VARIANT 7: note = G, H, Q or N
  VARIANT 8: note = K, I, F, M, D, Y, L, W, H or R
  VARIANT 10: note = H or L
  VARIANT 13: note = Q or E
XIXPXFXXAXYAXKFQG

SEQ ID NO: 52 - protein, 15 aa, synthetic construct
  VARIANT 1: note = R, K, F, N, H, P or Q
  VARIANT 7: note = S, W, R, L, Q, G, Y, D, N, A or M
  VARIANT 8: note = G, A, V, S, Q or N
  VARIANT 9: note = S or P
XKFHFVXXXPFGMDV

SEQ ID NO: 53 - protein, 440 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGGSQAVVTQEPSLTVSPGGTVTLTCRSSTGAVTTSNYANWVQQKPDHLPRALIGGTNKRAPGVPARFSGSLLGDKAALTISGAQPEDEAIYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 54 - protein, 123 aa, synthetic construct; identical to SEQ ID NO: 33

SEQ ID NO: 55 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSNSPFGMDVWGQGTTVTVSS

SEQ ID NO: 56 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSQSPFGMDVWGQGTTVTVSS

SEQ ID NO: 57 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVWGSPFGMDVWGQGTTVTVSS

SEQ ID NO: 58 - protein, 123 aa, synthetic construct
QVQLVQSGAEVKKPGSSVKVSCKTSGDTFSTYAISWVRQAPGQGLEWMGGIIPKFGKAHYAQKFQGRVTITADESTSTAYMELSSLRSEDTAVYFCARKFHFVSGSPFGMDVWGQGTTVTVSS

SEQ ID NO: 59 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSSSPFGMDVWGQGTTVTVSS

SEQ ID NO: 60 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVRGSPFGMDVWGQGTTVTVSS

SEQ ID NO: 61 - protein, 123 aa, synthetic construct
QVQLVQSGAEVKKPGSSVKVSCKTSGDTFSTYAISWVRQAPGQGLEWMGGIIPWFGKAHYAQKFQGRVTITADESTSTAYMELSSLRSEDTAVYFCARKFHFVSGSPFGMDVWGQGTTVTVSS

SEQ ID NO: 62 - protein, 123 aa, synthetic construct
QVQLVQSGAEVKKPGSSVKVSCKTSGDTFSTYAISWVRQAPGQGLEWMGGIIPIFGRAHYAQKFQGRVTITADESTSTAYMELSSLRSEDTAVYFCARKFHFVSGSPFGMDVWGQGTTVTVSS

SEQ ID NO: 63 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVLGSPFGMDVWGQGTTVTVSS

SEQ ID NO: 64 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVQGSPFGMDVWGQGTTVTVSS

SEQ ID NO: 65 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVGGSPFGMDVWGQGTTVTVSS

SEQ ID NO: 66 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVYGSPFGMDVWGQGTTVTVSS

SEQ ID NO: 67 - protein, 123 aa, synthetic construct
QVQLVQSGAEVKKPGSSVKVSCKTSGDTFSTYAISWVRQAPGQGLEWMGGIIPIFGHAHYAQKFQGRVTITADESTSTAYMELSSLRSEDTAVYFCARKFHFVSGSPFGMDVWGQGTTVTVSS

SEQ ID NO: 68 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVDGSPFGMDVWGQGTTVTVSS

SEQ ID NO: 69 - protein, 123 aa, synthetic construct
QVQLVQSGAEVKKPGSSVKVSCKTSGDTFSTYAISWVRQAPGQGLEWMGGIIPIFGWAHYAQKFQGRVTITADESTSTAYMELSSLRSEDTAVYFCARKFHFVSGSPFGMDVWGQGTTVTVSS

SEQ ID NO: 70 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSVSPFGMDVWGQGTTVTVSS

SEQ ID NO: 71 - protein, 123 aa, synthetic construct
QVQLVQSGAEVKKPGSSVKVSCKTSGDTFSTYAISWVRQAPGQGLEWMGGIIPLFGKAHYAQKFQGRVTITADESTSTAYMELSSLRSEDTAVYFCARKFHFVSGSPFGMDVWGQGTTVTVSS

SEQ ID NO: 72 - protein, 123 aa, synthetic construct
QVQLVQSGAEVKKPGSSVKVSCKTSGDTFSTYAISWVRQAPGQGLEWMGGIIPIFGLAHYAQKFQGRVTITADESTSTAYMELSSLRSEDTAVYFCARKFHFVSGSPFGMDVWGQGTTVTVSS

SEQ ID NO: 73 - protein, 123 aa, synthetic construct
QVQLVQSGAEVKKPGSSVKVSCKTSGDTFSTYLISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 74 - protein, 123 aa, synthetic construct
QVQLVQSGAEVKKPGSSVKVSCKTSGDTFSTYAISWVRQAPGQGLEWMGGIIPYFGKAHYAQKFQGRVTITADESTSTAYMELSSLRSEDTAVYFCARKFHFVSGSPFGMDVWGQGTTVTVSS

SEQ ID NO: 75 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSASPFGMDVWGQGTTVTVSS

SEQ ID NO: 76 - protein, 123 aa, synthetic construct
QVQLVQSGAEVKKPGSSVKVSCKTSGDTFSTYHISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 77 - protein, 123 aa, synthetic construct
QVQLVQSGAEVKKPGSSVKVSCKTSGDTFSTYAISWVRQAPGQGLEWMGGIIPIFHKAHYAQKFQGRVTITADESTSTAYMELSSLRSEDTAVYFCARKFHFVSGSPFGMDVWGQGTTVTVSS

SEQ ID NO: 78 - protein, 123 aa, synthetic construct
QVQLVQSGAEVKKPGSSVKVSCKTSGDTFSTYAISWVRQAPGQGLEWMGGIIPIFGYAHYAQKFQGRVTITADESTSTAYMELSSLRSEDTAVYFCARKFHFVSGSPFGMDVWGQGTTVTVSS

SEQ ID NO: 79 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCAQKFHFVSGSPFGMDVWGQGTTVTVSS

SEQ ID NO: 80 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCAPKFHFVSGSPFGMDVWGQGTTVTVSS

SEQ ID NO: 81 - protein, 123 aa, synthetic construct
QVQLVQSGAEVKKPGSSVKVSCKTSGDTFSTYAISWVRQAPGQGLEWMGGIIPIFGDAHYAQKFQGRVTITADESTSTAYMELSSLRSEDTAVYFCARKFHFVSGSPFGMDVWGQGTTVTVSS

SEQ ID NO: 82 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVNGSPFGMDVWGQGTTVTVSS

SEQ ID NO: 83 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCAHKFHFVSGSPFGMDVWGQGTTVTVSS

SEQ ID NO: 84 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCANKFHFVSGSPFGMDVWGQGTTVTVSS

SEQ ID NO: 85 - protein, 123 aa, synthetic construct
QVQLVQSGAEVKKPGSSVKVSCKTSGDTFSTYAISWVRQAPGQGLEWMGHIIPIFGKAHYAQKFQGRVTITADESTSTAYMELSSLRSEDTAVYFCARKFHFVSGSPFGMDVWGQGTTVTVSS

SEQ ID NO: 86 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVAGSPFGMDVWGQGTTVTVSS

SEQ ID NO: 87 - protein, 123 aa, synthetic construct
QVQLVQSGAEVKKPGSSVKVSCKTSGDTFSTYSISWVRQAPGQGLEWMGGIIPIFGKAHYAQKFQGRVTITADESTSTAYMELSSLRSEDTAVYFCARKFHFVAGSPFGMDVWGQGTTVTVSS

SEQ ID NO: 88 - protein, 123 aa, synthetic construct
QVQLVQSGAEVKKPGSSVKVSCKTSGDTFSTYAISWVRQAPGQGLEWMGGIIPIFGMAHYAQKFQGRVTITADESTSTAYMELSSLRSEDTAVYFCARKFHFVSGSPFGMDVWGQGTTVTVSS

SEQ ID NO: 89 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVMGSPFGMDVWGQGTTVTVSS

SEQ ID NO: 90 - protein, 123 aa, synthetic construct
QVQLVQSGAEVKKPGSSVKVSCKTSGDTFSTYAISWVRQAPGQGLEWMGNIIPIFGKAHYAQKFQGRVTITADESTSTAYMELSSLRSEDTAVYFCARKFHFVSGSPFGMDVWGQGTTVTVSS

SEQ ID NO: 91 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCAFKFHFVSGSPFGMDVWGQGTTVTVSS

SEQ ID NO: 92 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCAKKFHFVSGSPFGMDVWGQGTTVTVSS

SEQ ID NO: 93 - protein, 123 aa, synthetic construct
QVQLVQSGAEVKKPGSSVKVSCKTSGDTFSTYAISWVRQAPGQGLEWMGGIIPIFGFAHYAQKFQGRVTITADESTSTAYMELSSLRSEDTAVYFCARKFHFVSGSPFGMDVWGQGTTVTVSS

SEQ ID NO: 94 - protein, 123 aa, synthetic construct
QVQLVQSGAEVKKPGSSVKVSCKTSGDTFSTYAISWVRQAPGQGLEWMGGIIPIFGIAHYAQKFQGRVTITADESTSTAYMELSSLRSEDTAVYFCARKFHFVSGSPFGMDVWGQGTTVTVSS

SEQ ID NO: 95 - protein, 123 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGPPFGMDVWGQGTTVTVSS

SEQ ID NO: 96 - protein, 123 aa, synthetic construct
QVQLVQSGAEVKKPGSSVKVSCKTSGDTFSTYAISWVRQAPGQGLEWMGGIIPQFGKAHYAQKFQGRVTITADESTSTAYMELSSLRSEDTAVYFCARKFHFVSGSPFGMDVWGQGTTVTVSS

SEQ ID NO: 97 - protein, 123 aa, synthetic construct
QVQLVQSGAEVKKPGSSVKVSCKTSGDTFSTYAISWVRQAPGQGLEWMGGIIPIFQKAHYAQKFQGRVTITADESTSTAYMELSSLRSEDTAVYFCARKFHFVSGSPFGMDVWGQGTTVTVSS

SEQ ID NO: 98 - protein, 123 aa, synthetic construct
QVQLVQSGAEVKKPGSSVKVSCKTSGDTFSTYAISWVRQAPGQGLEWMGGIFPIFGKAHYAQKFQGRVTITADESTSTAYMELSSLRSEDTAVYFCARKFHFVSGSPFGMDVWGQGTTVTVSS

SEQ ID NO: 99 - protein, 123 aa, synthetic construct
QVQLVQSGAEVKKPGSSVKVSCKTSGDTFSTYAISWVRQAPGQGLEWMGGIIPIFGKALYAQKFQGRVTITADESTSTAYMELSSLRSEDTAVYFCARKFHFVSGSPFGMDVWGQGTTVTVSS

SEQ ID NO: 100 - protein, 123 aa, synthetic construct
QVQLVQSGAEVKKPGSSVKVSCKTSGDTFSTYAISWVRQAPGQGLEWMGGIIPRFGKAHYAQKFQGRVTITADESTSTAYMELSSLRSEDTAVYFCARKFHFVSGSPFGMDVWGQGTTVTVSS

SEQ ID NO: 101 - protein, 123 aa, synthetic construct
QVQLVQSGAEVKKPGSSVKVSCKTSGDTFSRYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 102 - protein, 123 aa, synthetic construct
QVQLVQSGAEVKKPGSSVKVSCKTSGDTFSQYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 103 - protein, 123 aa, synthetic construct
QVQLVQSGAEVKKPGSSVKVSCKTSGDTFSTYAISWVRQAPGQGLEWMGGIIPIFNKAHYAQKFQGRVTITADESTSTAYMELSSLRSEDTAVYFCARKFHFVSGSPFGMDVWGQGTTVTVSS

SEQ ID NO: 104 - protein, 123 aa, synthetic construct
QVQLVQSGAEVKKPGSSVKVSCKTSGDTFSTYAISWVRQAPGQGLEWMGGIIPIFGKAHYAEKFQGRVTITADESTSTAYMELSSLRSEDTAVYFCARKFHFVSGSPFGMDVWGQGTTVTVSS

SEQ ID NO: 105 - protein, 123 aa, synthetic construct
QVQLVQSGAEVKKPGSSVKVSCKTSGDTFSQYAISWVRQAPGQGLEWMGGIIPIFQKAHYAQKFQGRVTITADESTSTAYMELSSLRSEDTAVYFCARKFHFVRGSPFGMDVWGQGTTVTVSS

SEQ ID NO: 106 - protein, 123 aa, synthetic construct
QVQLVQSGAEVKKPGSSVKVSCKTSGDTFSTYAISWVRQAPGQGLEWMGGIIPIFQKAHYAQKFQGRVTITADESTSTAYMELSSLRSEDTAVYFCARKFHFVRGSPFGMDVWGQGTTVTVSS

SEQ ID NO: 107 - protein, 123 aa, synthetic construct
QVQLVQSGAEVKKPGSSVKVSCKTSGDTFSQYAISWVRQAPGQGLEWMGGIIPIFGKAHYAQKFQGRVTITADESTSTAYMELSSLRSEDTAVYFCARKFHFVRGSPFGMDVWGQGTTVTVSS

SEQ ID NO: 108 - protein, 439 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGSQAVVTQEPSLTVSPGGTVTLTCRSSTGAVTTSNYANWVQQKPDHLPRALIGGTNKRAPGTPARFSGSLLGDKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 109 - protein, 440 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGGSQAVVTQEPSLTVSPGGTVTLTCRSSTGAVTTSNYANWVQQKPDHLPRALIGGTNKRAPGTPARFSGSLLGDKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 110 - protein, 439 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGSQTVVTQEPSLTVSPGGTVTLTCRSSTGAVTTSNYPNWVQQKPGQAPRALIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 111 - protein, 439 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGSQAVVTQEPSLTVSPGGTVTLTCRSSTGAVTTSNYANWVQQKPDHLPRALIGGTNKRAPGVPARFSGSLLGDKAALTISGAQPEDEAIYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 112 - protein, 439 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGSQAVVTQEPSLTVSPGGTVTLTCRSSTGAVTSGYYANWVQQKPDHLPRALIGGTNKRAPGTPARFSGSLLGDKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 113 - protein, 439 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGSQTVVTQEPSLTVSPGGTVTLTCRSSTGAVTSGYYPNWVQQKPGQAPRALIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 114 - protein, 439 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 115 - protein, 439 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 116 - protein, 692 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSEVQLVESGGGLVQPGGSLRLSCAASGFTFNTYAMNWVRQAPGKGLEWVGRIRSKYNNYATYYADSVKDRFTISRDDSQSILYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 693 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGGSEVQLVESGGGLVQPGGSLRLSCAASGFTFNTYAMNWVRQAPGKGLEWVGRIRSKYNNYATYYADSVKDRFTISRDDSQSILYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 692 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSEVQLVESGGGLVQPGGSLRLSCAASGFTFNTYAMNWVRQAPGKGLEWVARIRSKYNNYATYYADSVKDRFTISRDDSQSILYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 693 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGGSEVQLVESGGGLVQPGGSLRLSCAASGFTFNTYAMNWVRQAPGKGLEWVARIRSKYNNYATYYADSVKDRFTISRDDSQSILYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 692 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSEVQLVESGGGLVQPGGSLRLSCAASGFTFNTYAMNWVRQAPGKGLEWVGRIRSKYNNYATYYADSVKGRFTISRDDSKNSLYLQMNSLKTEDTAVYYCARHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 692 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCARHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 692 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 692 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSEVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 223 aa, synthetic construct
SCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 125 - protein, 439 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLRGGSQAVVTQEPSLTVSPGGTVTLTCRSSTGAVTTSNYANWVQQKPDHLPRALIGGTNKRAPGTPARFSGSLLGDKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 126 - protein, 440 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLRGGGSQAVVTQEPSLTVSPGGTVTLTCRSSTGAVTTSNYANWVQQKPDHLPRALIGGTNKRAPGTPARFSGSLLGDKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 127 - protein, 439 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLRGGSQAVVTQEPSLTVSPGGTVTLTCRSSTGAVTTSNYANWVQQKPDHLPRALIGGTNKRAPGVPARFSGSLLGDKAALTISGAQPEDEAIYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 128 - protein, 440 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLRGGGSQAVVTQEPSLTVSPGGTVTLTCRSSTGAVTTSNYANWVQQKPDHLPRALIGGTNKRAPGVPARFSGSLLGDKAALTISGAQPEDEAIYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 129 - protein, 692 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPLGLRGGSEVQLVESGGGLVQPGGSLRLSCAASGFTFNTYAMNWVRQAPGKGLEWVARIRSKYNNYATYYADSVKDRFTISRDDSQSILYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 693 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPLGLRGGGSEVQLVESGGGLVQPGGSLRLSCAASGFTFNTYAMNWVRQAPGKGLEWVARIRSKYNNYATYYADSVKDRFTISRDDSQSILYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 692 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPLGLRGGSEVQLVESGGGLVQPGGSLRLSCAASGFTFNTYAMNWVRQAPGKGLEWVGRIRSKYNNYATYYADSVKGRFTISRDDSKNSLYLQMNSLKTEDTAVYYCARHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 693 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPLGLRGGGSEVQLVESGGGLVQPGGSLRLSCAASGFTFNTYAMNWVRQAPGKGLEWVGRIRSKYNNYATYYADSVKGRFTISRDDSKNSLYLQMNSLKTEDTAVYYCARHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 661 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGSQAVVTQEPSLTVSPGGTVTLTCRSSTGAVTTSNYANWVQQKPDHLPRALIGGTNKRAPGTPARFSGSLLGDKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 134 - protein, 662 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGGSQAVVTQEPSLTVSPGGTVTLTCRSSTGAVTTSNYANWVQQKPDHLPRALIGGTNKRAPGTPARFSGSLLGDKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 135 - protein, 661 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGSQAVVTQEPSLTVSPGGTVTLTCRSSTGAVTTSNYANWVQQKPDHLPRALIGGTNKRAPGVPARFSGSLLGDKAALTISGAQPEDEAIYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 136 - protein, 662 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGGSQAVVTQEPSLTVSPGGTVTLTCRSSTGAVTTSNYANWVQQKPDHLPRALIGGTNKRAPGVPARFSGSLLGDKAALTISGAQPEDEAIYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 137 - protein, 688 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSEVQLVESGGGLVQPGGSLRLSCAASGFTFNTYAMNWVRQAPGKGLEWVARIRSKYNNYATYYADSVKDRFTISRDDSQSILYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 689 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGGSEVQLVESGGGLVQPGGSLRLSCAASGFTFNTYAMNWVRQAPGKGLEWVARIRSKYNNYATYYADSVKDRFTISRDDSQSILYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 688 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSEVQLVESGGGLVQPGGSLRLSCAASGFTFNTYAMNWVRQAPGKGLEWVGRIRSKYNNYATYYADSVKGRFTISRDDSKNSLYLQMNSLKTEDTAVYYCARHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 689 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGGSEVQLVESGGGLVQPGGSLRLSCAASGFTFNTYAMNWVRQAPGKGLEWVGRIRSKYNNYATYYADSVKGRFTISRDDSKNSLYLQMNSLKTEDTAVYYCARHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 472 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGGSEVQLVESGGGLVQPGGSLRLSCAASGFTFNTYAMNWVRQAPGKGLEWVARIRSKYNNYATYYADSVKDRFTISRDDSQSILYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSGGGGSC

SEQ ID NO: 142 - protein, 453 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPLGLGGGGSQAVVTQEPSLTVSPGGTVTLTCRSSTGAVTTSNYANWVQQKPDHLPRALIGGTNKRAPGTPARFSGSLLGDKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 143 - protein, 680 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPELLGGGGSEVQLVESGGGLVQPGGSLRLSCAASGFTFNTYAMNWVRQAPGKGLEWVARIRSKYNNYATYYADSVKDRFTISRDDSQSILYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 452 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPLGLGGGSQAVVTQEPSLTVSPGGTVTLTCRSSTGAVTTSNYANWVQQKPDHLPRALIGGTNKRAPGVPARFSGSLLGDKAALTISGAQPEDEAIYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 145 - protein, 679 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPELLGGGSEVQLVESGGGLVQPGGSLRLSCAASGFTFNTYAMNWVRQAPGKGLEWVGRIRSKYNNYATYYADSVKGRFTISRDDSKNSLYLQMNSLKTEDTAVYYCARHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 453 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPLGLRGGGSQAVVTQEPSLTVSPGGTVTLTCRSSTGAVTTSNYANWVQQKPDHLPRALIGGTNKRAPGTPARFSGSLLGDKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 147 - protein, 693 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGGSEVQLVESGGGLVQPGGSLRLSCAASGFTFNTYAMNWVRQAPGKGLEWVGRIRSKYNNYATYYADSVKGRFTISRDDSKNSLYLQMNSLKTEDTAVYYCARHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 452 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPLGLRGGSQAVVTQEPSLTVSPGGTVTLTCRSSTGAVTTSNYANWVQQKPDHLPRALIGGTNKRAPGVPARFSGSLLGDKAALTISGAQPEDEAIYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 149 - skipped

SEQ ID NO: 150 - protein, 453 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGGSQAVVTQEPSLTVSPGGTVTLTCRSSTGAVTTSNYANWVQQKPDHLPRALIGGTNKRAPGTPARFSGSLLGDKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 151 - protein, 680 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLRGGGSEVQLVESGGGLVQPGGSLRLSCAASGFTFNTYAMNWVRQAPGKGLEWVARIRSKYNNYATYYADSVKDRFTISRDDSQSILYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 452 aa, synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSQAVVTQEPSLTVSPGGTVTLTCRSSTGAVTTSNYANWVQQKPDHLPRALIGGTNKRAPGVPARFSGSLLGDKAALTISGAQPEDEAIYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 153 - protein, 680 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLRGGGSEVQLVESGGGLVQPGGSLRLSCAASGFTFNTYAMNWVRQAPGKGLEWVGRIRSKYNNYATYYADSVKGRFTISRDDSKNSLYLQMNSLKTEDTAVYYCARHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK